(19) United States
(12) Patent Application Publication
(10) Pub. No.: US 2023/0175081 A1
(43) Pub. Date: Jun. 8, 2023

(54) MEANS AND METHODS FOR DETECTING NOVEL CORONAVIRUS (SARS-COV-2)

(71) Applicant: MULTIPLEXDX, S.R.O., Bratislava (SK)

(72) Inventors: Pavol CEKAN, Bratislava (SK); Monika RADVÁNSZKA, Bratislava (SK); Roman HAJDU, Bratislava (SK); Evan D. PAUL, Bratislava (SK)

(73) Assignee: MULTIPLEXDX, S.R.O., Bratislava (SK)

(21) Appl. No.: 17/997,996

(22) PCT Filed: May 4, 2021
(86) PCT No.: PCT/EP2021/061742

§ 371 Date: Nov. 4, 2022

(30) Foreign Application Priority Data
May 4, 2020 (EP) 20172733.6

(51) Int. Cl. C12Q 1/70; C12Q 1/686

(52) U.S. Cl. CPC C12Q 1/701; C12Q 1/686; C12Q 2600/16

(57) ABSTRACT

The present invention relates to a PCR-method comprising conducting a simultaneous amplification step with at least primer nucleotide sequences for SARS-CoV-2 RdRP gene, at least primer nucleotide sequences for SARS-CoV-2 E gene and/or at least primer nucleotide sequences for human RNase P gene. Said PCR-method may further comprise a conducting an amplification step, preferably a simultaneous amplification step, with at least primer nucleotide sequences for a unique spike RNA. Also provided is a kit comprising primers and optionally probes to carry out the PCR-methods of the invention.

### SEQUENCE LISTING

This application contains a Sequence Listing in computer readable form, which is incorporated herein by reference.

### FIELD OF THE INVENTION

The present invention relates to means and methods in the field of diagnosis for SARS-CoV-2. Accordingly, provided by the present invention is a PCR-method comprising conducting a simultaneous amplification step with at least primer nucleotide sequences for SARS-CoV-2 RdRP gene, at least primer nucleotide sequences for SARS-CoV-2 E gene and at least primer nucleotide sequences for human RNase P gene. Said PCR-method may further comprise a conducting an amplification step, preferably a simultaneous amplification step, with at least primer nucleotide sequences for a unique spike RNA.

The present invention also provides a kit comprising primers and probes to carry out the PCR-methods of the invention. The invention further relates to the use of primer nucleotide sequences for SARS-CoV-2 RdRP gene, primer nucleotide sequences for SARS-CoV-2 E gene and primer nucleotide sequences for human RNase P gene for the (i) in vitro detection of SARS-CoV-2 in a sample, (ii) in vitro detection of an infection of a subject with SARS-CoV-2, (iii) in vitro detection of a contamination of a blood sample with SARS-CoV-2, or (iv) monitoring the therapy of SARS-CoV-2 in vitro.

The present invention further relates to means and methods for detecting influenza A (IAV) and B (IBV) viruses and/or differentiating SARS-CoV-2 from the most common seasonal influenzas (e.g., IAV and/or IBV).

The PCR methods, kits and uses of the present invention overcome uneven amplification/folding, false negatives, poor sensitivity and specificity and/or preferential amplification of certain specific targets, primer dimers known to be associated with multiplexing PCR-methods, kits or uses as well as reduce false-positive and false-negative read outs.

### BACKGROUND OF THE INVENTION

As reported by the World Health Organization (WHO), the WHO China Country Office was informed of cases of pneumonia of unknown etiology in Wuhan City, Hubei Province, on 31 Dec. 2019. A novel coronavirus currently termed 2019-nCoV was officially announced. The genome sequences suggest presence of a virus closely related to the members of a viral species termed severe acute respiratory syndrome (SARS)-related CoV, a species defined by the agent of the 2002 March outbreak of SARS in humans. The outbreak has resulted in thousands of confirmed human infections in multiple provinces throughout China before spreading to Europe and the United States. Cases of asymptomatic infection, mild illness, severe illness, and deaths have been reported. WHO declared the outbreak of 2019-nCoV to be a pandemic affecting the entire world and thus millions of people.

Among the foremost priorities to facilitate public health interventions is reliable laboratory diagnosis. In order to provide researchers and clinicians with protocols for the diagnosis of 2019-nCoV, WHO published different protocols applied in Europe, the United States, Japan, China, Hong Kong or Thailand (https://www.who.int/docs/default-source/coronaviruse/whoinhouseassays.pdf?sfvrsn=de3a76aa_2).

All of them are based on nucleic acid amplification in order to detect 2019-nCoV, if present in a sample. The protocols published by WHO use different gene targets of 2019-nCoV. For example, China CDC goes for ORF1ab and N as gene targets; Institut Pasteur, France, goes for two gene targets within RdRP; US CDC goes for the detection of three targets within the viral N gene; National Institute of Infectious Diseases, Japan, goes for pancorona and multiple targets as well as the gene encoding the spike protein; HKU, Hong Kong SAR goes for ORF1b-nsp14 and N gene; National Institute of Health, Thailand goes for N gene and Charité, Germany goes for RdRP and N gene. All these protocols apply quantitative/real-time RT-PCR assays (qRT-PCR).

However, some of these assays may have limitations as was assessed by Vogels et al. (2020); Cold Spring Harbor Laboratory, BMJ Yale (https://doi.org/10.1101/2020.03.30.20048108). Yet, with their comparative study, Vogels at al. aimed at assisting other laboratories in selecting appropriate assays for the detection of 2019-nCoV, meanwhile named SARS-CoV-2 (Gorbalenya et al. (2020), Nat. Microbiol. (5)4, 536-544, doi: 10.1038/s41564-020-0695-z), since they observed that several primer-probe sets applied in qRT-PCR may cross-react with SARS-CoV-2-negative nasopharyngeal swabs, others may be somewhat unspecific, etc.

A comprehensive SARS-CoV-2 testing strategy is an important tool for countries to mitigate the spread of the coronavirus disease 2019 (COVID-19) by facilitating early detection and implementation of appropriate epidemiological measures. The gold standard for identifying SARS-CoV-2 entails using RT-qPCR to detect the presence of one or more viral genes in a biological specimen. This method has unparalleled sensitivity, detecting down to single copies of viral RNA in a reaction, and can readily be deployed in diagnostic laboratories. Shortly after the publication of the first SARS-CoV-2 genome sequences, several reference laboratories and public health authorities provided the first publicly available RT-qPCR protocols. These protocols were instrumental in allowing countries to rapidly implement comprehensive testing strategies and often served as the backbone for commercial development of more streamlined tests with additional innovations. Currently, hundreds of RT-qPCR tests have been developed to detect SARS-CoV-2 and studies comparing the efficacy of these tests reveal important differences in the specimen input, gene targets, testing workflow, specificity, and sensitivity.

The RT-qPCR test developed by the Charité Institute of Virology in Berlin was one of the first protocols to be published (Corman et al. (2020), Euro Surveillance 25(3)) and shared by the WHO and was widely used throughout Europe during the early stages of the pandemic. At the time of development, few SARS-CoV-2 sequences were publicly available and there was a scarcity of viral isolates and positive patient samples, so the authors designed an initial screening assay that intentionally cross-reacts with SARS-CoV viral RNA (from the 2003 outbreak). A second confirmation assay targeting the RdRP gene contained two probes that differentiate SARS-CoV-2 from SARS-CoV. The RdRP primers and SARS-CoV-2-specific probe, however, contained several degenerate bases in areas thought to display genetic variability. The authors also pointed out the design of the RdRP reverse primer could reduce reaction efficiency due to its low predicted melting temperature (Corman V M, Drosten C. Authors' response: SARS-CoV-2 detection by real-time RT-PCR. Euro Surveill. 2020 May 28). While this protocol provided unequivocal benefits in the early phase of diagnostic testing, a variety of issues emerged regarding the performance of this test, primarily reduced sensitivity of the RdRP assay (Nalla A K et al. 2020, Comparative Performance of SARS-CoV-2 Detection Assays Using Seven Different Primer-Probe Sets and One Assay Kit. Journal of Clinical Microbiology; Vogels CBF et al. 2020, Analytical sensitivity and efficiency comparisons of SARS-CoV-2 RT-qPCR primer-probe sets. Nature Microbiology. 2020 October; 5(10):1299-305; Jung Y, et al., 2020 Comparative Analysis of Primer-Probe Sets for RT-qPCR of COVID-19 Causative Virus (SARS-CoV-2). ACS Infect Dis. 2020 Aug. 11; Pillonel T, et al., 2020. Letter to the editor: SARS-CoV-2 detection by real-time RT-PCR. Euro Surveill. 2020 May 28; Etievant S, et al., 2020 Performance Assessment of SARS-CoV-2 PCR Assays Developed by WHO Referral Laboratories. J Clin Med [Internet]. 2020 Jun. 16).

Accordingly, despite the protocols published by WHO or others, e.g., Corman et al. (2020), Euro Surveillance 25(3), doi: 10.2807/1560-7917.ES.2020.25.3.2000045), there is still a need for improved reliable and accurate diagnostic assays to prompt clinical and public health interventions.

The technical problem underlying the present application is thus to comply with this need. The technical problem is solved by providing the embodiments reflected in the claims, described in the description and illustrated in the examples and figures that follow.

Briefly, based on the original Charité protocol, several improved RT-qPCR were developed in the course of the present invention that address the limitations of the original protocol. Furthermore, significant improvements were made to the assays by incorporating an internal control, streamlining the test workflow by multiplexing, and developing technical innovations such as dual probes to increase both sensitivity and specificity of the respective PCR methods. Additional assays to detect influenza A and B were developed providing a useful diagnostic tool to differentiate SARS-CoV-2 from the most common seasonal influenzas. Some of the means and methods of the present invention are room temperature-stable for up to one month, providing one of the few RT-qPCR means/methods that eliminates the need for cold chain shipping and storage.

### SUMMARY OF THE INVENTION

Accordingly, in one aspect the present invention relates to a PCR-method comprising conducting a simultaneous amplification step with at least primer nucleotide sequences for SARS-CoV-2 RdRP, at least primer nucleotide sequences for SARS-CoV-2 E gene and at least primer nucleotide sequences for human RNase P.

In a further aspect the present invention relates to a PCR-method comprising

(i) a first PCR-method comprising conducting a simultaneous amplification step with at least primer nucleotide sequences for SARS-CoV-2 RdRP and at least primer nucleotide sequences for human RNase P; and/or, with “and” being preferred,

(ii) a second PCR-method comprising conducting a simultaneous amplification step with at least primer nucleotide sequences for SARS-CoV-2 E gene and at least primer nucleotide sequences for human RNase P,

wherein the same source material suspected to comprise SARS-CoV-2 nucleic acids used is used for the PCR of (i) and (ii).

In a yet further aspect, the present invention relates to a PCR-method comprising

(i) a first PCR-method comprising conducting a simultaneous amplification step with at least primer nucleotide sequences for SARS-CoV2 E gene and at least primer nucleotide sequences for a unique spike RNA;

(ii) a second PCR-method comprising conducting a simultaneous amplification step with at least primer nucleotide sequences for SARS-CoV-2 RdRP gene and at least primer nucleotide sequences for human RNase P; and

(iii) a third PCR-method comprising conducting a simultaneous amplification step with at least primer nucleotide sequences for SARS-CoV-2 RdRP gene, at least primer nucleotide sequences for SARS-CoV-2 E gene, at least primer nucleotide sequences for human RNase P and at least primer nucleotide sequences for a unique spike RNA and further comprising a unique RNA for SARS-CoV-2 RdRP gene, a unique RNA for SARS-CoV-2 E gene, a unique RNA for human RNase P.

wherein the same source material suspected to comprise SARS-CoV-2 nucleic acids is used for the PCR-method of (i) and (ii), and

wherein the PCR-method of (iii) further comprises as positive control a ribonucleic acid for SARS-CoV-2 RdRP gene, a ribonucleic acid for SARS-CoV-2 E gene, a ribonucleic acid for human RNase P and a ribonucleic acid for unique spike RNA.

Moreover, the present invention provides for a PCR-method comprising conducting an amplification step with at least the primer nucleotide sequences set forth in SEQ ID NOs: 1 and 2.

The present invention also relates to a kit comprising at least the primer nucleotide sequences set forth in SEQ ID NOs: 1 and 2, optionally a probe comprising the nucleotide sequence set forth in SEQ ID NO: 3 and optionally means for carrying out a PCR amplification step.

Finally, the present invention relates to the use of the primer nucleotide sequences set forth in SEQ ID NOs: 1 and 2 for the

(i) in vitro detection of SARS-CoV-2 in a sample,

(ii) in vitro detection of an infection of a subject with SARS-CoV-2,

(iii) in vitro detection of a contamination of a blood sample with SARS-CoV-2, or

(iv) monitoring the therapy of SARS-CoV-2 in vitro.

### BRIEF DESCRIPTION OF THE NUCLEOTIDE SEQUENCES

SEQ ID NO: 1:
5′-GTGAAATGGTCATGTGTGGCGG

forward primer nucleotide sequence for SARS-CoV-2 RdRP

SEQ ID NO 2:
5′-CGTGACAGCTTGACAAATGTTAAAAAC

reverse primer nucleotide sequence for SARS-CoV-2 RdRP

SEQ ID NO: 3:
5′-CAGGTGGAACCTCATCAGGAGATGC

probe for SARS-CoV-2 RdRP, comprises preferably FAM or HEX or YY at its 5′-end and preferably BHQ1 or BHQ2 at its 3′-end

SEQ ID NO: 4:
5′-ACAGGTACGTTAATAGTTAATAGCGT

forward primer nucleotide sequence for SARS-CoV-2 E gene

SEQ ID NO: 5:
5′-ATATTGCAGCAGTACGCACACA

reverse primer nucleotide sequence for SARS-CoV-2 E gene

SEQ ID NO: 6:
5′-ACACTAGCCATCCTTACTGCGCTTCG

probe for SARS-CoV-2 E gene, comprises preferably FAM or HEX or YY at its 5′-end and preferably BHQ1 or BHQ2 at its 3′-end

SEQ ID NO: 7:
5′-AGATTTGGACCTGCGAGCG

forward primer nucleotide sequence for human RNase P

SEQ ID NO: 8:
5′-GAGCGGCTGTCTCCACAAGT

reverse primer nucleotide sequence for human RNase P

SEQ ID NO: 9:
5′-TTCTGACCTGAAGGCTCTGCGCG

probe for human RNase P, comprises preferably HEX or YY or Cy5 at its 5′-end and preferably BHQ1 or BHQ2 or BHQ3 at its 3′-end

SEQ ID NO: 10:
5′ rArGrUrGrArArArUrGrGrUrCrArUrGrUrGrUrGrGrCrGrG
rArCrCrArGrGrUrGrGrArArCrCrUrCrArUrCrArGrGrArGrAr
UrGrCrCrArGrUrUrUrUrUrArArCrArUrUrUrGrUrCrArArGrC
rUrGrUrCrArCrGrG

positive control ribonucleotide for SARS-CoV-2 RdRP (“r” stands for “ribo(nucleotide”)

SEQ ID NO: 11:
5′ rArGrArCrArGrGrUrArCrGrUrUrArArUrArGrUrUrArArU
rArGrCrGrUrUrUrArCrArCrUrArGrCrCrArUrCrCrUrUrArCr
UrGrCrGrCrUrUrCrGrArUrUrGrUrGrUrGrCrGrUrArCrUrGrC
rUrGrCrArArUrArU

positive control ribonucleotide for SARS-CoV-2 E gene (“r” stands for “ribo(nucleotide”)

SEQ ID NO: 12:
5′ rGrCrArGrArUrUrUrGrGrArCrCrUrGrCrGrArGrCrGrGrGr
UrUrCrUrGrArCrCrUrGrArArGrGrCrUrCrUrGrCrGrCrGrGrAr
CrUrUrGrUrGrGrArGrArCrArGrCrCrGrCrUrC

positive control ribonucleotide for human RNase P (“r” stands for “ribo(nucleotide”)

SEQ ID NO: 13: 5′-CAAATGTTAAAAACACTATTAGCATA

reverse primer nucleotide sequence for SARS-CoV-2 RdRP

SEQ ID NO: 14: 5′-GTGARATGGTCATGTGTGGCGG

forward primer nucleotide sequence for SARS-CoV-2 RdRP (compare with SEQ ID NO. 1, in which the degenerate nucleotide “R” was converted to nucleotide “A”)

SEQ ID NO: 15: 5′-CARATGTTAAASACACTATTAGCATA

reverse primer nucleotide sequence for SARS-CoV-2 RdRP (compare with SEQ ID NO: 13, in which the degenerate nucleotides “R” and “S” were converted to nucleotide “A”)

SEQ ID NO: 16: 5′-CCAGGTGGWACRTCATCMGGTGATGC

probe for SARS-CoV-2 E gene, comprises preferably FAM at its 5′-end and preferably BHQ1 at its 3′-end (compare with SEQ ID NO: 3, in which the degenerate nucleotide “W” was converted to the nucleotide “A”, the degenerate nucleotide “R” to the nucleotide “C” and the degenerate nucleotide “M” to the nucleotide “A”)

SEQ ID NO: 17: 5′-ATGCAGTGCCACATTATGCAG

forward primer nucleotide sequence for unique spike RNA

SEQ ID NO: 18: 5′-AGCACATGTAGTGCCACTGG

reverse primer nucleotide sequence for unique spike RNA

SEQ ID NO: 19: 5′- CCACGGTTACATCCAGTGGCACTACA

probe for unique spike RNA, comprises preferably HEX or YY or Cy5 at its 5′-end and preferably BHQ1 or BHQ2 or BHQ3 at its 3′-end

SEQ ID NO: 20:
5′ rUrArUrGrCrArGrUrGrCrCrArCrArUrUrArUrGrCrArGrUr
GrCrCrArCrGrGrUrUrArCrArUrCrCrArGrUrGrGrCrArCrUrAr
CrArUrGrUrGrCrCrArUrUrArCrArUrUrUrArCrArUrCrCrArGr
UrGrGrCrArCrUrArCrArUrGrUrGrCrU

(unique) spike RNA (“r” stands for “ribo(nucleotide”)

SEQ ID NO. 21
TATGCAGTGCCACATTATGCAGTGCCACGGTTACATCCAGTGGCACTACA
TGTGCCATTACATTTACATCCAGTGGCACTACATGTGCT

synthetic, e.g., bioinformatically designed nucleic acid sequence serving as template for (unique) spike RNA which does preferably not occur in human and SARS-CoV-2

SEQ ID NO: 22: 5′- GTACTCATTCGTTTCGGAAGAGACAG

forward primer nucleotide sequence for SARS-CoV-2 E gene

SEQ ID NO: 23:
5′ rArUrGrUrArCrUrCrArUrUrCrGrUrUrUrCrGrGrArArGrAr
CrArGrUrUrArCrArCrUrArGrCrCrArUrCrCrUrUrArCrUrGrCr
GrCrUrUrCrGrArUrUrGrUrGrUrGrCrGrUrArCrUrGrCrUrGrCr
ArArUrArU

positive control ribonucleotide for SARS-CoV-2 E gene (“r” stands for “ribo(nucleotide”)

| Primer (F or R), | |
|---|---|
| probe (P) name | |
| and SEQ ID NO | Sequence (5′ - 3′) and preferable fluorophore/quencher |
| SEQ ID NO: 24 | GTACTCATTCGTTTCGGAAGAGACAG |
| E_Sarbeco_F3 | |
| SEQ ID NO: 25 | ATATTGCAGCAGTACGCACACA |
| E_Sarbeco_R2 | |
| SEQ ID NO: 26 | ACACTAGCCATCCTTACTGCGCTTCG |
| E_Sarbeco_P1 | preferably comprises FAM at its 5′-end and preferably comprises |
| | BHQ1 at its 3′-end |
| SEQ ID NO: 27 | GTGAAATGGTCATGTGTGGCGG |
| RdRP_SARSr-F2 | |
| SEQ ID NO: 28 | CGTGACAGCTTGACAAATGTTAAAAAC |
| RdRP_SARSr-R2 | |
| SEQ ID NO: 29 | CAGGTGGAACCTCATCAGGAGATGC |
| RdRP_SARSr-P2 | preferably comprises FAM at its 5′-end and preferably comprises |
| | BHQ1 at its 3′-end |
| SEQ ID NO: 30 | AtGtACTCATTCGTTTCGGAAGA |
| E_Sarbeco_F7 | |
| with LNA-Ts (t) | |
| SEQ ID NO: 31 | ATATTGCAGCAGTACGCACACA |
| E_Sarbeco_R2 | |
| SEQ ID NO: 32 | ACACTAGCCATCCTTACTGCGCTTCG |
| E_Sarbeco_P1 | preferably comprises FAM at its 5′-end and preferably comprises |
| | BHQ1 at its 3′-end |
| SEQ ID NO: 33 | GTGAAATGGTCATGTGTGGCGG |
| RdRP_SARSr-F2 | |
| SEQ ID NO: 34 | CGTGACAGCTTGACAAATGTTAAAAAC |
| RdRP_SARSr-R2 | |
| SEQ ID NO: 35 | TCAGGAGATGCCACAACTGCTTATGC |
| RdRP_SARSr-P8 | preferably comprises FAM at its 5′-end and preferably comprises |
| | BHQ1 at its 3′-end |
| SEQ ID NO: 36 | AGATTTGGACCTGCGAGCG |
| RNase P Forward | |
| SEQ ID NO: 37 | GAGCGGCTGTCTCCACAAGT |
| RNase P Reverse | |
| SEQ ID NO: 38 | TTCTGACCTGAAGGCTCTGCGCG |
| RNase P Probe 1 | preferably comprises FAM at its 5′-end and preferably comprises |
| | BHQ1 at its 3′-end |
| SEQ ID NO: 39 | AtGtACTCATTCGTTTCGGAAGA |
| E_Sarbeco_F7 | |
| with LNA-Ts (t) | |
| SEQ ID NO: 40 | ATATTGCAGCAGTACGCACACA |
| E_Sarbeco_R2 | |
| SEQ ID NO: 41 | ACACTAGCCATCCTTACTGCGCTTCG |
| E_Sarbeco_P1 | preferably comprises FAM at its 5′-end and preferably comprises |
| | BHQ1 at its 3′-end |
| SEQ ID NO: 42 | TAGCGTACTTCTTTTTCTTGCTTTCGTGGT |
| E_Sarbeco_P2 | preferably comprises FAM at its 5′-end and preferably comprises |
| | BHQ1 at its 3′-end |
| SEQ ID NO: 43 | GTGAAATGGTCATGTGTGGCGG |
| RdRP_SARSr-F2 | |
| SEQ ID NO: 44 | CGTGACAGCTTGACAAATGTTAAAAAC |
| RdRP_SARSr-R2 | |
| SEQ ID NO: 45 | CAGGTGGAACCTCATCAGGAGATGC |
| RdRP_SARSr-P2 | preferably comprises FAM at its 5′-end and preferably comprises |
| | BHQ1 at its 3′-end |
| SEQ ID NO: 46 | TCAGGAGATGCCACAACTGCTTATGC |
| RdRP_SARSr-P8 | preferably comprises FAM at its 5′-end and preferably comprises |
| | BHQ1 at its 3′-end |
| SEQ ID NO: 47 | AGATTTGGACCTGCGAGCG |
| RNase P Forward | |
| SEQ ID NO: 48 | GAGCGGCTGTCTCCACAAGT |
| RNase P Reverse | |
| SEQ ID NO: 49 | TTCTGACCTGAAGGCTCTGCGCG |
| RNase P Probe 1 | preferably comprises FAM at its 5′-end and preferably comprises |
| | BHQ1 at its 3′-end |
| SEQ ID NO: 50 | AtGtACTCATTCGTTTCGGAAGA |
| E_Sarbeco_F7 | |
| with LNA-Ts (t) | |
| SEQ ID NO: 51 | ATATTGCAGCAGTACGCACACA |
| E_Sarbeco_R2 | |
| SEQ ID NO: 52 | ACACTAGCCATCCTTACTGCGCTTCG |
| E_Sarbeco_P1 | preferably comprises FAM at its 5′-end and preferably comprises |
| | BHQ1 at its 3′-end |
| SEQ ID NO: 53 | TAGCGTACTTCTTTTTCTTGCTTTCGTGGT |
| E_Sarbeco_P2 | preferably comprises FAM at its 5′-end and preferably comprises |
| | BHQ1 at its 3′-end |
| SEQ ID NO: 54 | GTGAAATGGTCATGTGTGGCGG |
| RdRP_SARSr-F2 | |
| SEQ ID NO: 55 | CGTGACAGCTTGACAAATGTTAAAAAC |
| RdRP_SARSr-R2 | |
| SEQ ID NO: 56 | CAGGTGGAACCTCATCAGGAGATGC |
| RdRP_SARSr-P2 | preferably comprises FAM at its 5′-end and preferably comprises |
| | BHQ1 at its 3′-end |
| SEQ ID NO: 57 | TCAGGAGATGCCACAACTGCTTATGC |
| RdRP_SARSr-P8 | preferably comprises FAM at its 5′-end and preferably comprises |
| | BHQ1 at its 3′-end |
| SEQ ID NO: 58 | AGATTTGGACCTGCGAGCG |
| RNase P Forward | |
| SEQ ID NO: 59 | GAGCGGCTGTCTCCACAAGT |
| RNase P Reverse | |
| SEQ ID NO: 60 | TTCTGACCTGAAGGCTCTGCGCG |
| RNase P Probe 1.1 | preferably comprises YY at its 5′-end and preferably comprises |
| | BHQ2 at its 3′-end |
| SEQ ID NO: 61 | AtGtACTCATTCGTTTCGGAAGA |
| E_Sarbeco_F7 | |
| with LNA-Ts (t) | |
| SEQ ID NO: 62 | ATATTGCAGCAGTACGCACACA |
| E_Sarbeco_R2 | |
| SEQ ID NO: 63 | ACACTAGCCATCCTTACTGCGCTTCG |
| E_Sarbeco_P1.2 | preferably comprises YY at its 5′-end and preferably comprises |
| | BHQ2 at its 3′-end |
| SEQ ID NO: 64 | TAGCGTACTTCTTTTTCTTGCTTTCGTGGT |
| E_Sarbeco_P2.2 | preferably comprises YY at its 5′-end and preferably comprises |
| | BHQ2 at its 3′-end |
| SEQ ID NO: 65 | GTGAAATGGTCATGTGTGGCGG |
| RdRP_SARSr-F2 | |
| SEQ ID NO: 66 | CGTGACAGCTTGACAAATGTTAAAAAC |
| RdRP_SARSr-R2 | |
| SEQ ID NO: 67 | CAGGTGGAACCTCATCAGGAGATGC |
| RdRP_SARSr-P2 | preferably comprises FAM at its 5′-end and preferably comprises |
| | BHQ1 at its 3′-end |
| SEQ ID NO: 68 | TCAGGAGATGCCACAACTGCTTATGC |
| RdRP_SARSr-P8 | preferably comprises FAM at its 5′-end and preferably comprises |
| | BHQ1 at its 3′-end |
| SEQ ID NO: 69 | AGATTTGGACCTGCGAGCG |
| RNase P Forward | |
| SEQ ID NO: 70 | GAGCGGCTGTCTCCACAAGT |
| RNase P Reverse | |
| SEQ ID NO: 71 | TTCTGACCTGAAGGCTCTGCGCG |
| RNase P Probe 1.2 | preferably comprises Cy5 at its 5′-end and preferably comprises |
| | BHQ3 at its 3′-end |
| SEQ ID NO: 72 | AtGtACTCATTCGTTTCGGAAGA |
| E_Sarbeco_F7 | |
| with LNA-Ts (t) | |
| SEQ ID NO: 73 | ATATTGCAGCAGTACGCACACA |
| E_Sarbeco_R2 | |
| SEQ ID NO: 74 | ACACTAGCCATCCTTACTGCGCTTCG |
| E_Sarbeco_P1 | preferably comprises FAM at its 5′-end and preferably comprises |
| | BHQ1 at its 3′-end |
| SEQ ID NO: 75 | TAGCGTACTTCTTTTTCTTGCTTTCGTGGT |
| E_Sarbeco_P2 | preferably comprises FAM at its 5′-end and preferably comprises |
| | BHQ1 at its 3′-end |
| SEQ ID NO: 76 | GTGAAATGGTCATGTGTGGCGG |
| RdRP_SARSr-F2 | |
| SEQ ID NO: 77 | CGTGACAGCTTGACAAATGTTAAAAAC |
| RdRP_SARSr-R2 | |
| SEQ ID NO: 78 | CAGGTGGAACCTCATCAGGAGATGC |
| RdRP_SARSr-P2 | preferably comprises FAM at its 5′-end and preferably comprises |
| | BHQ1 at its 3′-end |
| SEQ ID NO: 79 | TCAGGAGATGCCACAACTGCTTATGC |
| RdRP_SARSr-P8 | preferably comprises FAM at its 5′-end and preferably comprises |
| | BHQ1 at its 3′-end |
| SEQ ID NO: 80 | AGATTTGGACCTGCGAGCG |
| RNase P Forward | |
| SEQ ID NO: 81 | GAGCGGCTGTCTCCACAAGT |
| RNase P Reverse | |
| SEQ ID NO: 82 | TTCTGACCTGAAGGCTCTGCGCG |
| RNase P Probe 1.2 | preferably comprises Cy5 at its 5′-end and preferably comprises |
| | BHQ3 at its 3′-end |
| SEQ ID NO: 83 | TTCTAGCATGGTGGAGGCCAT |
| IAV-F1.1 | |
| SEQ ID NO: 84 | CCGTCTGAGTTCTTCAATGGTGG |
| IAV-R1.2 | |
| SEQ ID NO: 85 | TCTAGGGCCCGGATTGATGCCA |
| IAV-Probe1.2 | preferably comprises YY at its 5′-end and preferably comprises |
| | BHQ2 at its 3′-end |
| SEQ ID NO: 86 | AGTGGACTCAGGAAAGTGGC |
| IBV-F2.1 | |
| SEQ ID NO: 87 | TCCATtTGtTGCATTGATTGAAGC |
| IBV-R2.3 with LNA- | |
| Ts (t) | |
| SEQ ID NO: 88 | TCCaAATGAAaTGGGGAATGGAAGCT |
| IBV-Probe2.3 with | preferably comprises YY at its 5′-end and preferably comprises |
| LNA-As (a) | BHQ2 at its 3′-end |
| Primer (F or R), | |
| probe (P) name | Sequence (5′ - 3′) and preferable fluorophore/quencher |
| and SEQ ID NO | (t) LNA-T; (a) LNA-A |
| SEQ ID NO: 89 | ACAGGTACGTTAATAGTTAATAGCGT |
| E_Sarbeco_F1 | |
| SEQ ID NO: 90 | CTCATTCGTTTCGGAAGAGACAGG |
| E_Sarbeco_F2 | |
| SEQ ID NO: 91 | GTACTCATTCGTTTCGGAAGAGACAG |
| E_Sarbeco_F3 | |
| SEQ ID NO: 92 | CGTTTCGGAAGAGACAGGTACG |
| E_Sarbeco_F4 | |
| SEQ ID NO: 93 | TGTACTCATTCGTTTCGGAAGAGACA |
| E_Sarbeco_F5 | |
| SEQ ID NO: 94 | TGTACTCATTCGTTTCGGAAGAGACAG |
| E_Sarbeco_F6 | |
| SEQ ID NO: 95 | AtGtACTCATTCGTTTCGGAAGA |
| E_Sarbeco_F7 | |
| SEQ ID NO: 96 | AtGtACTCATTCGTTTCGGAAGAC |
| E_Sarbeco_F8 | |
| SEQ ID NO: 97 | ATGtACtCAtTCGTTTCGGA |
| E_Sarbeco_F9 | |
| SEQ ID NO: 98 | AtGtACtCATTCGTTTCGGA |
| E_Sarbeco_F10 | |
| SEQ ID NO: 99 | ATGtACTCAtTCGTTtCGGA |
| E_Sarbeco_F11 | |
| SEQ ID NO: 100 | AtGTACTCATtCGTTtCGGA |
| E_Sarbeco_F12 | |
| SEQ ID NO: 101 | ATGtACTCAtTCGTtTCGGA |
| E_Sarbeco_F13 | |
| SEQ ID NO: 102 | ATATTGCAGCAGTACGCACACA |
| E_Sarbeco_R2 | |
| SEQ ID NO: 103 | ACACTAGCCATCCTTACTGCGCTTCG |
| E_Sarbeco_P1 | preferably comprises FAM or HEX or YY at its 5′-end and preferably |
| | comprises BHQ1 or BHQ2 at its 3′-end |
| SEQ ID NO: 104 | TAGCGTACTTCTTTTTCTTGCTTTCGTGGT |
| E_Sarbeco_P2 | preferably comprises FAM or HEX or YY at its 5′-end and preferably |
| | comprises BHQ1 or BHQ2 at its 3′-end |
| SEQ ID NO: 105 | CGAAGCGCAGTAAGGATGGCTAGTGT |
| Sarbeco_P1rev | preferably comprises FAM at its 5′-end and preferably comprises |
| | BHQ1 at its 3′-end |
| SEQ ID NO: 106 | TAGCGTACTTCTTtTTCTTGCTTTCGTGGT |
| E_Sarbeco_P3 | preferably comprises FAM at its 5′-end and preferably comprises |
| | BHQ1 at its 3′-end |
| SEQ ID NO: 107 | TAGCGTACTTCTtTtTCTTGCTTTCGTGGT |
| E_Sarbeco_P4 | preferably comprises FAM at its 5′-end and preferably comprises |
| | BHQ1 at its 3′-end |
| SEQ ID NO: 108 | TAGCGTACTTCTTtTTCTTGCTTTCGTGGT |
| E_Sarbeco_P5 | preferably comprises HEX at its 5′-end and preferably comprises |
| | BHQ2 at its 3′-end |
| SEQ ID NO: 109 | GTGAAATGGTCATGTGTGGCGG |
| RdRP_SARSr-F2 | |
| SEQ ID NO: 110 | CGTGACAGCTTGACAAATGTTAAAAAC |
| RdRP_SARSr-R2 | |
| SEQ ID NO: 111 | CGTGACAGCTTGACAAATGTtAAAAAC |
| RdRP_SARSr-R4 | |
| SEQ ID NO: 112 | CGTGACAGCTTGACAAAtGTtAAAAAC |
| RdRP_SARSr-R5 | |
| SEQ ID NO: 113 | CAGGTGGAACCTCATCAGGAGATGC |
| RdRP_SARSr-P2 | preferably comprises FAM or HEX or YY at its 5′-end and preferably |
| | comprises BHQ1 or BHQ2 at its 3′-end |
| SEQ ID NO: 114 | CAGGTGGAACCTCATCAGGAGATGC |
| RdRP_SARSr-P3 | preferably comprises FAM at its 5′-end and preferably comprises |
| | BHQ1 at its 3′-end and preferably comprises BHQ1 at T at position |
| | 12 |
| SEQ ID NO: 115 | GTGGAACCTCATCAGGAGATGCCAC |
| RdRP_SARSr-P4 | preferably comprises FAM at its 5′-end and preferably comprises |
| | BHQ1 at its 3′-end |
| SEQ ID NO: 116 | GTGGAACCTCATCAGGAGATGCCAC |
| RdRP_SARSr-P5 | preferably comprises FAM at its 5′-end and preferably comprises |
| | BHQ1 at its 3′-end and preferably comprises BHQ1 at T at position 9 |
| SEQ ID NO: 117 | GGAACCTCATCAGGAGATGCCACAAC |
| RdRP_SARSr-P6 | preferably comprises FAM at its 5′-end and preferably comprises |
| | BHQ1 at its 3′-end |
| SEQ ID NO: 118 | GGAACCTCATCAGGAGATGCCACAAC |
| RdRP_SARSr-P7 | preferably comprises FAM at its 5′-end and preferably comprises |
| | BHQ1 at its 3′-end and preferably comprises BHQ1 at T at position |
| | 10 |
| SEQ ID NO: 119 | TCAGGAGATGCCACAACTGCTTATGC |
| RdRP_SARSr-P8 | preferably comprises FAM or HEX or YY at its 5′-end and preferably |
| | comprises BHQ1 or BHQ2 at its 3′-end |
| SEQ ID NO: 120 | TCAGGAGATGCCACAACTGCTTATGC |
| RdRP_SARSr-P9 | preferably comprises FAM at its 5′-end and preferably comprises |
| | BHQ1 at its 3′-end and preferably comprises BHQ1 at T at position 9 |
| SEQ ID NO: 121 | TTCTAGCATGGTGGAGGCCAT |
| IAV-F1.1 | |
| SEQ ID NO: 122 | ATTTCTAGCATGGTGGAGGCC |
| IAV-F1.2 | |
| SEQ ID NO: 123 | AATCCCCTGAATCCCTTTGT |
| IAV-F2.1 | |
| SEQ ID NO: 124 | AaTCCCCTGAaTCCCTTTGT |
| IAV-F2.2 | |
| SEQ ID NO: 125 | AAtCCCCTGAAtCCCTTTGT |
| IAV-F2.3 | |
| SEQ ID NO: 126 | CGTCTGAGTTCTTCAATGGTGG |
| IAV-R1.1 | |
| SEQ ID NO: 127 | CCGTCTGAGTTCTTCAATGGTGG |
| IAV-R1.2 | |
| SEQ ID NO: 128 | TCTGGCATCAATCCGGGC |
| IAV-R2.1 | |
| SEQ ID NO: 129 | AGGGCCCGGATTGATGCCAGA |
| IAV-Probe1.1 | |
| SEQ ID NO: 130 | TCTAGGGCCCGGATTGATGCCA |
| IAV-Probe1.2 | preferably comprises FAM or HEX or YY at its 5′-end and preferably |
| | comprises BHQ1 or BHQ2 at its 3′-end |
| SEQ ID NO: 131 | AAGAGtTCTCTGaGATCAtGAaGATC |
| IAV-Probe1.3 | |
| SEQ ID NO: 132 | AAGCAATGCCAGCATGGGAA |
| IBV-F1.1 | |
| SEQ ID NO: 133 | TGaATGAaAGCAATGCCAGCA |
| IBV-F1.2 | |
| SEQ ID NO: 134 | GaATGaAAGCAATGCCAGCA |
| IBV-F1.3 | |
| SEQ ID NO: 135 | AGTGGACTCAGGAAAGTGGC |
| IBV-F2.1 | |
| SEQ ID NO: 136 | TTtAGGATtGGCTCCCTATTTGT |
| IBV-F2.2 | |
| SEQ ID NO: 137 | TTtAGGATtGGCTCCCTATTTGTG |
| IBV-F2.3 | |
| SEQ ID NO: 138 | ACTGTGTTTAGGATTGGCTCCCTA |
| IBV-F2.4 | |
| SEQ ID NO: 139 | GGGAGCCAATCCTAAACACAGT |
| IBV-R1.1 | |
| SEQ ID NO: 140 | GaGCCAaTCCTAAACACAGT |
| IBV-R1.2 | |
| SEQ ID NO: 141 | GaGCCAaTCCTaAACACAGT |
| IBV-R1.3 | |
| SEQ ID NO: 142 | TCCATTTGTTGCATTGATTGAAGC |
| IBV-R2.1 | |
| SEQ ID NO: 143 | TCCATTTGTTGCATTGATTGAAGCA |
| IBV-R2.2 | |
| SEQ ID NO: 144 | TCCATtTGtTGCATTGATTGAAGC |
| IBV-R2.3 | |
| SEQ ID NO: 145 | AGAGTGGACTCAGGAAAGTGGCC |
| IBV-Probe1.1 | |
| SEQ ID NO: 146 | CAGAGTGGACTCAGGAAAGTGGCC |
| IBV-Probe1.2 | |
| SEQ ID NO: 147 | TCCAAATGAAATGGGGAATGGAAGCT |
| IBV-Probe2.1 | |
| SEQ ID NO: 148 | AGATCCAAATGAAATGGGGAATGGAAGCT |
| IBV-Probe2.2 | |
| SEQ ID NO: 149 | TCCaAATGAAaTGGGGAATGGAAGCT |
| IBV-Probe2.3 | preferably comprises FAM or HEX or YY at its 5′-end and preferably |
| | comprises BHQ1 or BHQ2 at its 3′-end |
| SEQ ID NO: 150 | TGCAGAGTGAATGGCACAAATAAGATCCA |
| IBV-Probe2.4 | |
| SEQ ID NO: 151 | TGCCGAGTGAATGGCACAAATAAGATCC |
| IBV-Probe2.5 | |
| SEQ ID NO: 152 | ACTGTGTTTAGGATTGGCTCCCTATTTGTG |
| IBV-Probe2.6 | |
| SEQ ID NO: 153 | CTGtGTTTAGGATtGGCTCCCTAtT |
| IBV-Probe2.7 | |
| SEQ ID NO: 154 | AGATTTGGACCTGCGAGCG |
| RNase P Forward | |
| SEQ ID NO: 155 | GAGCGGCTGTCTCCACAAGT |
| RNase P Reverse | |
| SEQ ID NO: 156 | TTCTGACCTGAAGGCTCTGCGCG |
| RNase P Probe | preferably comprises FAM or HEX or YY or Cy5 at its 5′-end and |
| | preferably comprises BHQ1 or BHQ2 or BHQ3 at its 3′-end |
| Primer (F or R), | |
| probe (P) name | Sequence (5′ - 3′) |
| and SEQ ID NO | (t) LNA-T; (a) LNA-A |
| SEQ ID NO: 157 | TCTtTtCCAATGTTACTTGGTTC |
| SARS-CoV-2 S | |
| gene - F1 | |
| SEQ ID NO: 158 | AGTAGGGACTGGGTCTTCGAATCT |
| SARS-CoV-2 S | |
| gene - R1 | |
| SEQ ID NO: 159 | GTtACTtGGTTCCATGCTATCTC |
| B.1.1.7- F3 | |
| SEQ ID NO: 160 | CAACtTTtGTTGTTTTTGtGGTAAGC |
| B.1.1.7 - R36 | |
| SEQ ID NO: 161 | |
| SARS-CoV-2 S | AGAGGTTTGATAACCCTGtCCtACCA |
| gene / | |
| B.1.1.7 - P3 | |
| SEQ ID NO: 162 | TtTGCTTCCACTGAGAAGTCtAACAT |
| SARS-CoV-2 S | |
| gene / | |
| B.1.1.7- P4 | |

Nucleotides in lowercase and bold denote LNA-modified bases
F, forward primer; P, probe; R, reverse primer

### BRIEF DESCRIPTION OF THE FIGURES

FIG. 1: RT-PCR for detection of SARS CoV-2 genes RdRP, E and human RNase P. The figure shows the detection of SARS-CoV-2 E gene and SARS-CoV-2 RdRP gene by real-time RT-PCR. Curve with filled circles shows the amplification reaction for SARS-CoV-2 gene using primer nucleotide sequences of SEQ ID NOs: 4 and 5 and a probe having SEQ ID NO: 6. Curve with filled squares shows the amplification reaction for SARS-CoV-2 gene using primer nucleotide sequences of SEQ ID NOs: 1 and 13 together with the probe of SEQ ID NO: 3. Curve with filled triangles and filled diamonds shows the amplification reaction for SARS-CoV-2 gene using primer nucleotide sequences of SEQ ID NOs: 1 and 2, together with the probe of SEQ ID NO: 3. Curve with filled stars shows the amplification reaction for SARS-CoV-2 gene using primer nucleotide sequences of SEQ ID NOs: 14 and 15 and a probe of SEQ ID NO: 16. Curve with open diamonds shows the amplification reaction for SARS-CoV-2 gene using primer nucleotide sequences of SEQ ID NOs: 14 and 15 and the probe of SEQ ID NO: 3. Curve with open squares shows the amplification reaction for SARS-CoV-2 gene using primer nucleotide sequences of SEQ ID NOs: 1 and 2, but no probe was used.

FIG. 2: Schematic illustrating SARS-CoV-2 genome and regions targeted by RT-qPCR primers and probes. A) Schematic overview portrays the SARS-CoV-2 genome with RdRP and E gene regions magnified to show the locations of primers and probes of the original Charité protocol, vDetect (v1 and v2), and rTest RT-qPCR assays. F, forward primer; P, probe; R, reverse primer. B) Diagram compares the sequences of primers and probes for the original Charité protocol, vDetect (v1 and v2), and rTest RT-qPCR assays to the Wuhan reference sequence. Magenta lines and letters represent mixed bases found in the primers and probes in the Charité protocol that were replaced with the correct bases in vDetect v1 (blue lines and letters).

FIG. 3: Redesign and validation of Charité SARS-CoV-2 E and RdRP primer/probe sets. A) Performance of redesigned RdRP gene reverse primers with replaced mixed bases and optimized melting temperatures. B) Heatmap shows various E gene forward primers with and without LNA-modified thymine residues (LNA-T) and their relative performance amplifying SARS-CoV-2 template or samples contaminated with E gene synthetic positive control. C) Limit of detection of both E (left panel) and RdRP (right panel) gene assays. Dotted line at Ct=40 denotes the detection cutoff. ND, not detected. D) Clinical evaluation of both vDetect v.1 E (left panel) and RdRP (right panel) gene assays conducted in two independent laboratories. Dotted lines at ND and shaded areas show detection cutoff and samples that were not detected for either the vDetect v.1 assay, index test assay, or both assays. Ct, cycle threshold; E, envelope gene; R, reverse primer; RdRP, RNA-dependent RNA polymerase; NTC, no template control.

FIG. 4: Evaluation of dual probes, analytical sensitivity, and clinical performance of rTEST COVID-19 qPCR. Comparison of analytical sensitivity (A, C) and fluorescent intensity (B, D) between single probes versus dual probes for both RdRP (A, B) and E (C, D) genes. Analytical limit of detection was performed in triplicates using the SARS-CoV-2 Standard (Exact Diagnostics) diluted to the desired concentrations. Dotted line at Ct 40 serves as a threshold after which amplification is considered invalid. ND indicates samples that were not detected. *p<0.05, **p<0.01, ***p<0.001, ****p<0.0001. E, F) Clinical performance of the RdRP gene (E) and E gene (F) assays in the rTEST COVID-19 qPCR kit compared to an index test (vDetect v.1) used in routine clinical practice. The dotted lines and shaded areas indicate samples that were not detected by either the evaluation test, index test, or both tests. Ct, cycle threshold; E, envelope gene; P, probe; RdRP, RNA-dependent RNA polymerase; NTC, no template control.

FIG. 5: Analytical sensitivity and clinical performance of multiplexed RT-qPCR assays. A, B) Graphs depict the analytical sensitivity of the multiplexed E and RNase P assay (A) and multiplexed RdRP and RNase P assay (B) in the rTEST Covid-19 qPCR Multiplex kit. C) Clinical performance of the rTEST Covid-19 qPCR Multiplex kit. D) Analytical sensitivity of the triplexed E, RdRP, and RNase P assay in the rTEST Covid-19 qPCR Allplex kit. E) Clinical performance of the rTEST Covid-19 qPCR Multiplex kit. The dotted line at Ct 40 (A, B, and D) serves as a threshold after which amplification is considered invalid. The dotted lines and shaded areas (C, E) indicate samples that were not detected by either the evaluation test, index test, or both tests. Ct, cycle threshold; E, envelope gene; RdRP, RNA-dependent RNA polymerase; NTC, no template control. ND indicates samples that were not detected.

FIG. 6: Schematic illustrating influenza A and B genome and regions targeted by RT-qPCR primers and probes. A) Schematic overview portrays the influenza A and B genome with PB1 and PA gene regions magnified to show the locations of primers and probes. Nucleotides labeled in red text indicate mixed bases in the consensus sequences for influenza A and B. BHQ2, black hole quencher 2; F, forward primer; HA, hemagglutinin; M, matrix protein; NA, neuraminidase; NP, nucleoprotein; NS, non-structural protein; P, probe; PA, polymerase acidic protein; PB1, polymerase basic 1 protein; PB2, polymerase basic 2 protein; R, reverse primer; seg., segment; YY, Yakima Yellow®.

FIG. 7: Analytical sensitivity and clinical performance of rTEST COVID-19/FLU qPCR kit. A, B) Graphs depict the analytical sensitivity of the multiplexed SARS-CoV-2 E, IAV PB1, and RNase P assay (A) and multiplexed SARS-CoV-2 RdRP, IBV PA, and RNase P assay (B) in the rTEST COVID-19/FLU qPCR kit. C) Analytical sensitivity of the multiplexed SARS-CoV-2 E and RdRP (both labeled with FAM), IAV PB1 and IBV PA (both labeled with YY), and RNase P assay. D) Clinical performance of the rTEST COVID-19/FLU qPCR kit. The dotted line at Ct 40 (A, B, and C) serves as a threshold after which amplification is considered invalid. The dotted line and shaded area (D) indicate samples that were not detected by a particular assay. Ct, cycle threshold; E, envelope gene; IAV, influenza A; IBV, influenza B; PA, polymerase acidic protein; PB1, polymerase basic 1 protein; NTC, no template control; RdRP, RNA-dependent RNA polymerase. ND indicates samples that were not detected.

FIG. 8: Optimization of vDetect Covid-19 qPCR kits. A) Heatmap shows the optimization of reverse transcription (RT) and annealing temperatures for the HighQu 1Step RT qPCR Probe ROX L Kit using RT-qPCR. B) Heatmap displays the parameters optimized for the Agilent Brilliant III Ultra-Fast QRT-PCR Master Mix using PCR followed by gel electrophoresis. C) Comparison of three different thermal profiles identified as being beneficial by PCR/gel electrophoresis (see B) using RT-qPCR. D) Assessment of higher RT concentration. E) Evaluation of analytical sensitivity (limit of detection) for E and RdRP assays of the vDetect v.2 COVID-19 RT-qPCR test. A/E, annealing/extension; Ct, cycle threshold; E, envelope gene; D, denaturation; DTT, dithiothreitol; ID, initial denaturation; RdRP, RNA-dependent RNA polymerase; NTC, no template control.

FIG. 9: Optimization of the room-temperature stable rTEST COVID-19 qPCR kit. A) Heatmap shows the optimization of thermocycling parameters for the SOLIS BioDyne SOLIScript® 1-step CoV Kit using PCR followed by gel electrophoresis. B) Comparison of four different thermal profiles using RT-qPCR. C, D) Graphs depict the effects of decoy nucleic acids (tRNA or ssDNA) or pure oligonucleotides (pure) on the stability of lyophilized primer/probes sets for RdRP (C) and E (D) genes over a one-month period. E) Performance of either fresh or an rTEST COVID-19 qPCR kit left at room temperature for 1-month on amplification of SARS-CoV-2 E and RdRP genes and human RNase P. A/E, annealing/extension; Ct, cycle threshold; E, envelope gene; D, denaturation; RdRP, RNA-dependent RNA polymerase; ssDNA, salmon sperm DNA; tRNA, baker's yeast transfer RNA.

FIG. 10: Optimization and validation of rTEST COVID-19 qPCR kit. A) Plot shows the performance of various RdRP gene probes with (open bars) and without (closed bars) internal quenchers on amplification (left axis, whisker plots) and normalized fluorescence (right axis, bar graphs). The standard probe (P2) is shown in dark gray, the best probe (P8) is shown in turquoise, other probes are shown in light gray. B) Graph depicts comparison of E gene probes on amplification threshold. The standard probe (P1) is shown as black symbols, the best probe (P1P2) is shown as magenta symbols, other probes are shown as light gray symbols. C) Analytical sensitivity of the singleplex E, RdRP, and RNase P assays in the rTEST Covid-19 qPCR kit. D) Clinical performance of vDetect v.1 kit using thawed and re-extracted RNA to assess RNA degradation before clinical performance evaluation of r rTEST Covid-19 qPCR kit.

### DETAILED DESCRIPTION OF THE INVENTION

Sensitive and accurate RT-qPCR tests are the primary diagnostic tools to identify SARS-CoV-2 infected patients. While many SARS-CoV-2 RT-qPCR tests are available, there are significant differences in test sensitivity, workflow (e.g., hands-on-time), gene targets, and other functionalities that users must consider. Several publicly available protocols shared by reference labs and public health authorities provide useful tools for SARS-CoV-2 diagnosis, but many have shortcomings related to sensitivity and laborious workflows. Here, we describe a series of improved SARS-CoV-2 RT-qPCR tests that are based on the protocol developed by the Charité Institute of Virology. Notably, none of the protocols published by WHO applies a simultaneous PCR-method (multiplex PCR-method) for detecting at least two different genes from SARS-CoV2. However, based on their vast experience with multiplex PCR-methods, the present inventors apply in one aspect of the present invention a simultaneous PCR-method for detecting SARS-CoV-2 E gene and RdRP gene as well as human RNase P as a control and found that multiplexing works well. As will be described hereinafter, the principle of multiplexing is applied in various other PCR-methods of the present invention described herein.

Accordingly, the present invention relates to a PCR-method comprising conducting a simultaneous amplification step with at least primer nucleotide sequences for SARS-CoV-2 RdRP gene, at least primer nucleotide sequences for SARS-CoV-2 E gene and at least primer nucleotide sequences for human RNase P gene.

PCR-methods are well known to those of ordinary skill in the art. In the PCR technique, a sample of DNA is mixed in a solution with a molar excess of at least two oligonucleotide primers of that are prepared to be complementary to the 3′ end of each strand of the DNA duplex; a molar excess of nucleotide bases (i.e., dNTPs); and a heat stable DNA polymerase, (preferably Taq polymerase), which catalyzes the formation of DNA from the oligonucleotide primers and dNTPs. Of the primers, at least one is a forward primer that will bind in the 5′ to 3′ direction to the 3′ end of one strand of the denatured DNA analyte and another is a reverse primer that will bind in the 3′ to 5′ direction to the 5′ end of the other strand of the denatured DNA analyte. The solution is heated to 94-96° C. to denature the double-stranded DNA to single-stranded DNA. When the solution cools down and reaches the so-called annealing temperature, the primers bind to separated strands and the DNA polymerase catalyzes a new strand of analyte by joining the dNTPs to the primers. When the process is repeated and the extension products synthesized from the primers are separated from their complements, each extension product serves as a template for a complementary extension product synthesized from the other primer. As the sequence being amplified doubles after each cycle, a theoretical amplification of a huge number of copies may be attained after repeating the process for a few hours; accordingly, extremely small quantities of DNA may be amplified using PCR in a relatively short period of time.

A preferred PCR-method of the present invention is a real-time reverse transcriptase PCR or “real time RT-PCR”. Sometimes it is also referred to as quantitative RT-PCR (qRT-PCR). Reverse transcription polymerase chain reaction (RT-PCR) is a known laboratory technique combining reverse transcription of RNA into cDNA and amplification of specific DNA targets using polymerase chain reaction (PCR defined elsewhere herein). It is primarily used to measure the amount of a specific RNA. This is achieved by monitoring the amplification reaction using fluorescence, a technique called real-time PCR or quantitative PCR (qPCR). Combined RT-PCR and qPCR are routinely used for analysis of gene expression and quantification of viral RNA in research and clinical settings. Said techniques are known to the skilled artisan. Briefly, the method relies on a DNA-based probe with a fluorescent reporter at one end and a quencher of fluorescence at the opposite end of the probe. In the context of the present invention, the fluorescence reporter is preferably HEX (hexachloro-fluoresceine) or FAM (Carboxyfluorescein) or YY (Yakima Yellow) or Cy 5 (cyanine 5) or ATTO-647N, while the quencher is preferably BHQ1 or BHQ2 or BHQ3 (Black Hole Quencher® Dyes). The close proximity of the reporter to the quencher prevents detection of its fluorescence; breakdown of the probe by the 5′ to 3′ exonuclease activity of the Taq polymerase breaks the reporter-quencher proximity and thus allows unquenched emission of fluorescence, which can be detected after excitation with a laser. An increase in the product targeted by the reporter probe at each PCR cycle therefore causes a proportional increase in fluorescence due to the breakdown of the probe and release of the reporter. General steps of a RT-qPCR are: RNA isolation, reverse transcription, followed by a PCR. Protocols for RNA isolation, reverse transcription and PCR are commonly known and, e.g., described in Corman et al. (2020), cited above or available at https://www.fda.gov/media/134922/download, https://www.who.int/docs/default-source/coronaviruse/protocol-v2-1. pdf?sfvrsn=a9ef618c_2.

(i) The PCR is prepared as usual (as defined elsewhere herein), and the reporter probe is added (ii) As the reaction commences, during the annealing stage of the PCR both probe and primers anneal to the nucleic acid target. (iii) Polymerisation of a new nucleic acid strand is initiated from the primers, and once the polymerase reaches the probe, its 5′-3′-exonuclease degrades the probe, physically separating the fluorescent reporter from the quencher, resulting in an increase in fluorescence. (iv) Fluorescence is detected and measured in a real-time PCR machine, and its geometric increase corresponding to exponential increase of the product is used to determine the quantification cycle (Cq) in each reaction.

A RT-PCR method of the invention can, for example, be carried out as described in Corman et al. 2020 (Corman V M, Landt O, Kaiser M Detection of 2019 novel coronavirus (2019-nCoV) by real-time RT-PCR), or in WHO-in house protocols available at https://www.who.int/docs/default source/coronaviruse/whoinhouseassays.pdf?sfvrsn=de3a76aa_2. In particular, the RNA from biological samples was obtained by using AMPIXTRACT™ SARS-CoV-2 Extraction Kit or Kit Extraction NucleoSpin Dx Virus or QIAmp DSP Viral RNA Mini Kit or EZ1 DSP Virus Kit or bioMerieux NucliSENS® easyMAG®, etc.

Preferably, the PCR-method may be a RT-LAMP. The Reverse transcription loop-mediated isothermal amplification (RT-LAMP) is a technique for the amplification of RNA. RT-LAMP does not require the typical PCR cycles and is performed at a constant temperature between 60 and 65° C. Similar to RT-PCR, RT-LAMP uses reverse transcriptase to synthesize complementary DNA (cDNA) from RNA sequences. This method can be very effective in detecting viruses with an RNA genome.

A PCR-method of the invention comprises preferably a simultaneous amplification step, in other words is a “multiplex PCR-method” or “multiplex assay”. As used herein, a multiplex assay may be an assay that is suitable to simultaneously amplify and identify different target nucleic acids of SARS-CoV-2. According to the present invention, a multiplex assay preferably screens simultaneously for SARS-CoV-2 RdRP gene, SARS-CoV-2 E gene and human RNase P gene. In this context the human RNase P gene is used as internal control.

It is also preferred that a multiplex assay screens for SARS-CoV-2 RdRP gene and SARS-CoV-2 E gene. It is also preferred that a multiplex assay screens for SARS-CoV-2 RdRP gene and human RNase P gene.

It is also preferred that a multiplex assay screens for SARS-CoV-2 E gene and human RNase P gene.

It is also preferred that a multiplex assay screens for SARS-CoV-2 RdRP gene and a unique spike RNA.

It is also preferred that a multiplex assay screens for SARS-CoV-2 E gene and a unique spike RNA.

It is also preferred that a multiplex assay screens for SARS-CoV-2 RdRP gene, SARS-CoV-E gene, human RNase P gene and a unique spike RNA.

In some aspects, the present invention relates to improved nucleotide sequences of primers and probes disclosed herein, particularly to dual probes disclosed herein, and improved PCR reaction conditions for enhancing the sensitivity and specificity of the means and methods of the present invention. By incorporating an RNase P internal control and developing multiplexed assays for distinguishing SARS-CoV-2 and influenza A and B, the present invention streamlines the PCR-workflow in order to provide quicker results and reduce the costs of the consumables. Preferably, means and methods of the present invention can use a room temperature-stable master mix and lyophilized positive control, thus increasing the functionality of the PCR-methods of the present invention and eliminating cold chain shipping and storage. The RT-qPCR methods of the present invention can easily be implemented in any diagnostic laboratory and can provide a powerful tool for detecting SARS-CoV-2 and the most common seasonal influenzas during the vaccination phase of the pandemic.

As described herein references can be made to UniProtKB Accession Numbers (http://www.uniprot.org/ e.g., as available in UniProt release 2021_01, published Feb. 10, 2021). As described herein references can be made to NCBI GenBank Accession Numbers (https://www.ncbi.nlm.nih.gov/genbanI</release/current/ e.g., as available in Release 242.0, published on Feb. 15, 2021).

A “primer nucleotide sequence” as used herein denotes an oligonucleotide which is used as primer or starter for a polymerase to synthesize a nucleic acid strand as is commonly known in the art. Sometimes herein, said term is abbreviated as “primer”, such as forward or reverse primer. Primers and probes were designed/synthesized by methods known in the art. Primers and probes for use in the PCR-methods of the present invention can be designed using, for example, a computer program such as OLIGO (Molecular Biology Insights, Inc., Cascade, Colo.). Important features when designing oligonucleotides to be used as amplification primers include, but are not limited to, an appropriate size amplification product to facilitate detection, similar melting temperatures for the members of a pair of primers, and the length of each primer (i.e., the primers need to be long enough to anneal with sequence-specificity and to initiate synthesis but not so long that fidelity is reduced during oligonucleotide synthesis). Typically, oligonucleotide primers are 15 to 30 nucleotides in length. Designing oligonucleotides to be used as probes can be performed in a manner similar to the design of primers, although the members of a pair of probes preferably anneal to an amplification product. As with oligonucleotide primers, oligonucleotide probes usually have similar melting temperatures, and the length of each probe must be sufficient for sequence-specific hybridization to occur but not so long that fidelity is reduced during synthesis. Oligonucleotide probes are generally 15 to 30 nucleotides in length. Primers useful within the context of the present invention include oligonucleotides suitable in PCR reactions for the amplification of nucleic acids derived from SARS-CoV-2 virus. In particular, in the context of the present invention primers of SEQ ID Nos: 1 and 2 are used for the amplification of the SARS-CoV-2 RdRP gene, primers of SEQ ID Nos: 4 or 22 and 5 are used for amplification of the SARS-CoV-2 E gene, primers of SEQ ID Nos: 7 and 8 are used to amplify human RNase P. Also in the context of the present invention, a probe comprising the nucleotide sequence of SEQ ID NO: 3 is used in combination with primers of SEQ ID NO: 1 and 2 for RdRP gene amplification, a probe comprising the nucleotide sequence of SEQ ID NO: 6 is used in combination with primers of SEQ ID Nos: 4 or 22 and 5 for E gene amplification, a probe comprising the nucleotide sequence of SEQ ID NO: 9 is used in combination with primers of SEQ ID Nos: 7 and 8 for human RNase P gene amplification, a probe comprising the nucleotide sequence of SEQ ID NO: 19 is used in combination with primers of SEQ ID Nos: 17 and 18 for unique spike RNA amplification.

As mentioned before, again, none of the protocols published by WHO applies the principle of multiplexing, particularly for a simultaneous PCR-method (multiplex PCR-method) for detecting at least one gene from SARS-CoV and a human gene as control.

Accordingly, in a further aspect, the present invention relates to a PCR-method comprising

(i) a first PCR-method comprising conducting a simultaneous amplification step with at least primer nucleotide sequences for SARS-CoV-2 RdRP and at least primer nucleotide sequences for human RNase P; and/or, with “and” being preferred,

(ii) a second PCR-method comprising conducting a simultaneous amplification step with at least primer nucleotide sequences for SARS-CoV-2 E gene and at least primer nucleotide sequences for human RNase P,

wherein the same source material suspected to comprise SARS-CoV-2 nucleic acids used is used for the PCR of (i) and (ii).

The term “source material” as used herein includes any material, preferably biological material, e.g., from a mammalian, particularly human or veterinary subject that may be tested for the presence or absence of SARS-CoV-2. For avoidance of doubt, source material after being processed results in a sample. Thus, the term “source material” encompasses the term “sample”. Samples may include, without limitation, tissues obtained from any organ, such as for example, lung tissue; and fluids such as for example, blood, plasma, serum, lymphatic fluid, synovial fluid, cerebrospinal fluid, amniotic fluid, amniotic cord blood, tears, throat or nasal swabs, saliva, oral rinses (e.g., gargle), and nasopharyngeal washes. Also stool, urine or sperm is included by the term “source material”. In the context of the present invention the source material may be sputum as well as nose and throat swabs obtained from patients with or without viral transport medium. A source material when being subject to a PCR-method of the present invention is prima facie suspected to comprise SARS-CoV-2 nucleic acids. Thus, it is preferred that a source material is subject to a step which provides for the isolation of nucleic acids, preferably RNA, more preferably viral RNA, in particular SARS-CoV-2 RNA.

The “same” source material means that an aliquot of one and the same source material, e.g., RNA isolated therefrom, is used for a PCR-method as described herein.

It is preferred that the first and second PCR-method is performed in parallel, i.e., under same conditions, e.g., using the same reagents, tools or device(s), etc.

Thus far, none of the WHO protocols provides for an assay including, in addition to the detection of SARS-CoV-2 genes, also simultaneous controls for the RNA isolation, swab control, positive control(s), etc. Ideally, the controls and the detection for SARS-CoV-2 gene(s) is done in one and the same method.

Accordingly, again by making use of simultaneous (multiplexing) PCR-methods, the present invention further envisages a PCR-method comprising

(i) a first PCR-method comprising conducting a simultaneous amplification step with at least primer nucleotide sequences for SARS-CoV2 E gene and at least primer nucleotide sequences for a unique spike RNA;

(ii) a second PCR-method comprising conducting a simultaneous amplification step with at least primer nucleotide sequences for SARS-CoV-2 RdRP gene and at least primer nucleotide sequences for human RNase P; and

(iii) a third PCR-method comprising conducting a simultaneous amplification step with at least primer nucleotide sequences for SARS-CoV-2 RdRP gene, at least primer nucleotide sequences for SARS-CoV-2 E gene, at least primer nucleotide sequences for human RNase P and at least primer nucleotide sequences for a unique spike RNA and further comprising a unique RNA for SARS-CoV-2 RdRP gene, a unique RNA for SARS-CoV-2 E gene, a unique RNA for human RNase P.

wherein the same source material suspected to comprise SARS-CoV-2 nucleic acids is used for the PCR-method of (i) and (ii), and

wherein the PCR-method of (iii) further comprises as positive control a ribonucleic acid for SARS-CoV-2 RdRP gene, a ribonucleic acid for SARS-CoV-2 E gene, a ribonucleic acid for human RNase P and a ribonucleic acid for unique spike RNA.

It is preferred that the first, second and third PCR-method is performed in parallel, i.e., under same conditions, e.g., using the same reagents, tools or device(s), etc.

A unique spike RNA as used herein refers to RNA of known sequence. It does preferably not occur in human and SARS-CoV-2. It may be designed based on bioinformatic data of human and SARS-CoV-2 nucleic acid sequences. If spiked into source material, it may be used in the PCR-methods of the present invention as control, e.g., whether or not RNA isolation worked, reaction conditions applied in PCR-methods worked, etc.

The unique spike used in the context of the present invention, in particular in the PCR-methods of the present invention is a ribonucleic acid having the sequence set forth in SEQ ID NO: 20. It may be transcribed from a nucleotide sequence shown in SEQ ID NO: 21.

The term “positive control” in its broadest use refers to the use of external DNA or RNA, with RNA being preferred, carrying a (target) gene of interest. If these positive controls are assayed in separate wells/tubes from the experimental sample, they serve, e.g., as a control to determine whether or not the reverse transcription and/or PCR reaction worked and, perhaps, whether the conditions applied were appropriate. Additionally, exogenous DNA or RNA, with RNA being preferred, may be spiked into the experimental sample(s), swabs, etc. and assayed, e.g., in parallel, or, preferably, in a multiplex format with the (target) gene of interest. These control reactions may assess, e.g., whether the samples or source material contain any components that inhibit reverse transcription and/or PCR or whether the isolation of nucleic acids, e.g., RNA from, e.g., a swab worked.

A positive control may be a nucleic acid sequence, in particular RNA from SARS-CoV-2. These are available, e.g., as synthetically derived viral RNA, said synthetically-derived viral RNA can be purchased, for example in form of kits like the EDX SARS-CoV-2 Standard (http://www.exactdiagnostics.com/sars-cov-2-standard.html). Alternatively, the SARS-CoV-2 may be synthetized by chemical methods, enzymatic methods or generated by molecular cloning or in vitro transcription see (https://www.twistbioscience.com/sites/default/files/resources/2020-03/Product%20Sheet%20_NGS_SyntheticSARS-CoV-2_RNAControls_17MAR20_Rev1.pdf. Alternatively, viral nucleic acid may be isolated from SARS-CoV-2 virus by methods known to the skilled artisan. For example, someone would use a commercially available kit that lyses the cells, separates the RNA via beads or a column, washes the RNA with salt and ethanol, and then elutes the purified RNA in water. For example, SARS-CoV-2 RNA as described before may also be used for setting-up and/or testing reaction conditions of the PCR-methods of the present invention.

In the context of the present invention said positive controls may preferably be a ribonucleic acid having the sequence set forth in SEQ ID NO: 10, a ribonucleic acid having the sequence set forth in SEQ ID NO: 11 or 23, a ribonucleic acid having the sequence set forth in SEQ ID NO: 12.

According to the present invention, a PCR-method for detecting SARS-CoV-2 virus nucleic acids in a biological sample that is more specific than particularly the assay from Charité, Germany (see, e.g., Corman et al. (2020), cited above) is described herein. Specifically, the present inventors observed that when performing the protocol from Charité, Germany (see Corman et al. (2020), cited above) the specificity, in particular for SARS-CoV-2 RdRP gene was not satisfying. As described hereinafter, the present inventors attempted to improve the protocol from Charité, Germany and they succeeded, inter alia, due to using different primer nucleotide sequences which significantly improved the specificity.

Namely, with respect to the detection of SARS-CoV-2 RdRP gene, the present inventors observed major specificity issues when using published primer nucleotide sequences (SEQ ID NOs: 14 and 15) and a probe (SEQ ID NO: 16)—well name “P1 eurofins” in Table 1. Indeed, they did not observe a Ct (dRn). Accordingly, the present inventors tackled this specificity issue in different ways and could successfully resolve it.

First, they converted all degenerate nucleotides of the probe having SEQ ID NO: 16 into non-degenerate, i.e., specific nucleotides, thereby obtaining the probe having SEQ ID NO: 3. Using primer nucleotide sequences (SEQ ID NOs: 14 and 15) and the probe having SEQ ID NO: 3—well name “P2 eurofins” in Table 1—the specificity (Ct (dRN)=34.26) was improved in contrast to “P1 eurofins” (no Ct (dRN) at all).

Second, since the specificity of 34.26 was still not satisfying, the present inventors converted the degenerate nucleotides of the primer nucleotide sequence of SEQ ID NOs: 14 and 15 into non-degenerate, i.e., specific nucleotides, thereby obtaining the primer nucleotide sequence having SEQ ID NOs: 1 and 13, respectively. Using these primer nucleotide sequences (SEQ ID NOs: 1 and 13) together with the probe having SEQ ID NO: 3—well name “R1 MDX” in Table 1—the specificity (Ct (dRN)=32.89) was further improved in contrast to “P2 eurofins” (Ct (dRN)=34.26).

Finally, in order to try to further improve the specificity of “R1 MDX” (see Table 1), the present inventors observed that the primer nucleotide sequences having SEQ ID NOs: 1 and 2, which when used together with the probe having SEQ ID NO: 3—well name “R2 MDX” and “R2 new MDX” in Table 1—confer for an even improved specificity (Ct (dRN)=31.17 and 31.18) in contrast to “R1 MDX” (Ct (dRN)=32.89) (see Table 1).

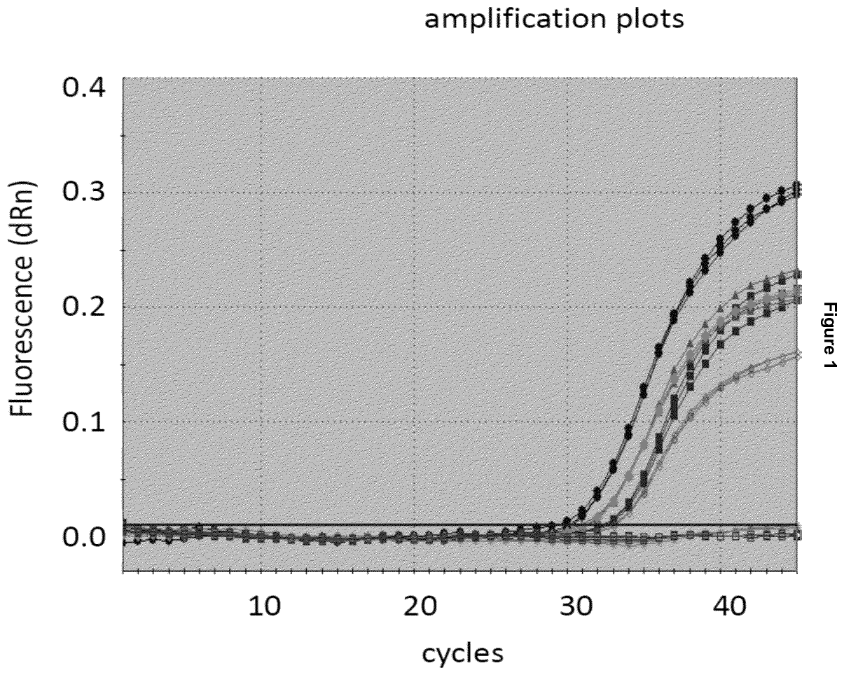

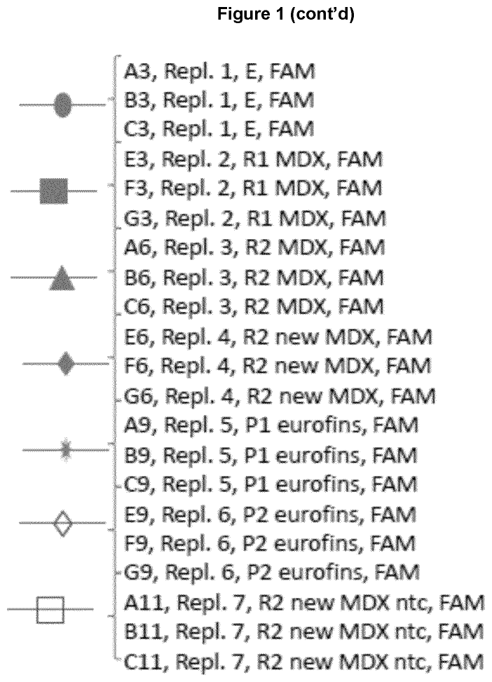

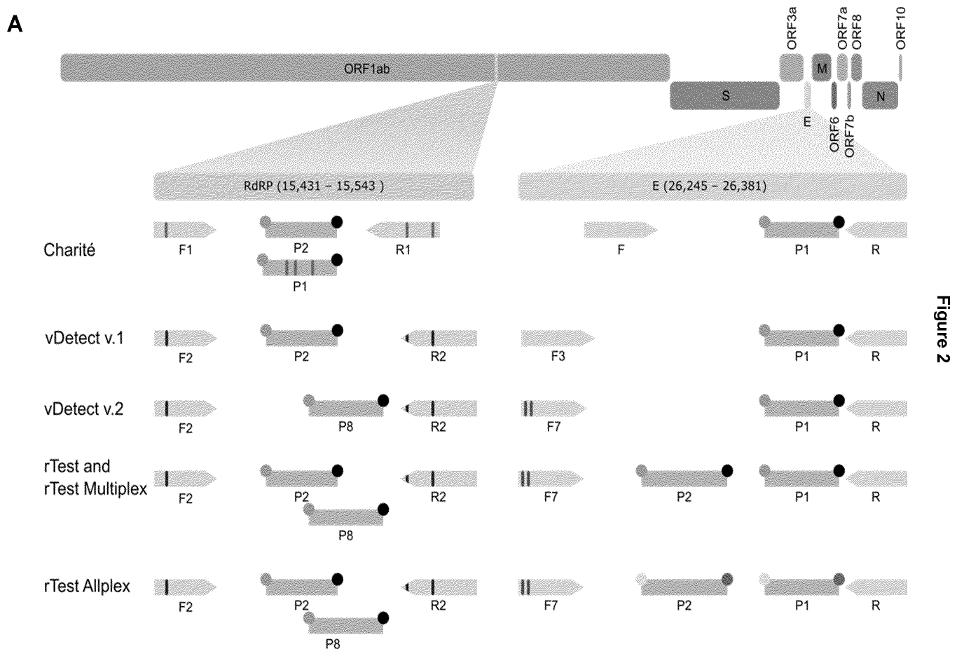

As a negative control, the primer nucleotide sequences having SEQ ID NOs: 1 and 2, but no probe was used—well name “R2 new MDX ntc”. The corresponding curve in FIG. 1 is marked by open squares.

Hence, the mere conversion of the degenerate primer nucleotide sequences and/or the degenerate probe from the Charité protocol (see, e.g., Corman et al. (2020), cited above) into non-degenerate primer nucleotide sequences was not sufficient to improve the detection of SARS-CoV-RdRP gene. It required more and, thus, the present inventors provide a newly designed reverse primer nucleotide sequence for the amplification step of the SARS-CoV-2 RdRP gene.

Accordingly, the present invention provides for a PCR-method comprising conducting an amplification step with at least the primer nucleotide sequences set forth in SEQ ID NOs: 1 and 2.

As explained before, the present inventors found that primer nucleotide sequences having SEQ ID NOs: 1 and 2, optionally together with a probe having SEQ ID NO: 3 provides for an improved specificity much improved specificity as regards the detection of SARS-CoV-2 RdRP gene vis-à-vis the commonly used primer nucleotide sequences having SEQ ID NOs: 14 and 15, optionally together with a probe having SEQ ID NO: 3 or 16. Indeed, comparing a Ct of 30.21 for SARS-CoV-2 E gene with a Ct of 31.17 and 31.18 for SARS-CoV-2 RdRP, it is apparent that there is a difference of less than one Ct between SARS-CoV-2 E gene and SARS-CoV-2 RdRP gene. This is a significant improvement of a method for the detection of SARS-CoV-2 as described in the present application.

Primers, in particular primer nucleotide sequences set forth in SEQ ID NO: 1 and 2, and probes for detecting SARS-CoV-2 as well as kits containing such primers and/or probes are also provided.

The increased sensitivity of PCR-method for detection of SARS-CoV-2 as well as the improved features of PCR primers, makes feasible the implementation of this technology for an accurate and reliable diagnosis of SARS-CoV-2 infections.

When used herein, the terms “polynucleotide”, “nucleic acid” or “nucleic acid molecule” are to be construed synonymously. Generally, nucleic acid molecules may comprise inter alia DNA molecules (including cDNA, complementary DNA), RNA molecules (e.g., miRNA, mRNA, rRNA, tRNA, snRNA, siRNA, scRNA, snoRNA, and others as known in the art). Furthermore, the term “nucleic acid molecule” may refer to DNA or RNA or hybrids thereof or any modification thereof that is known in the art, e.g., locked nucleic acid (LNA) (see, e.g., U.S. Pat. Nos. 5,525,711, 4,711,955, 5,792,608 or EP 302175 for examples of modifications). The polynucleotide sequence may be single- or double-stranded, linear or circular, natural or synthetic, and without any size limitation. For instance, the polynucleotide sequence may be genomic DNA, cDNA, mitochondrial DNA, mRNA, antisense RNA, ribosomal RNA or a DNA encoding such RNAs or chimeroplasts (Gamper, Nucleic Acids Research, 2000, 28, 4332-4339). Said polynucleotide sequence may be in the form of a vector, plasmid or of viral DNA or RNA. Also described herein are nucleic acid molecules, which are complementary to the nucleic acid molecules described above and nucleic acid molecules, which are able to hybridize to nucleic acid molecules described herein. A nucleic acid molecule described herein may also be a fragment of the nucleic acid molecules in context of the present invention. Particularly, such a fragment is a functional fragment. Examples for such functional fragments are nucleic acid molecules, which can serve as primers.

A “gene” from SARS-CoV-2 when used herein, e.g., in the context of SARS-CoV-2 E gene or SARS-CoV-2 RdRP gene is meant a nucleotide sequence, preferably RNA, such as SARS-CoV-2 E RNA or SARS-CoV-2 RdRP RNA, which comprises an open reading frame (ORF) encoding SARS-CoV-2 E protein or SARS-CoV-2 RdRP protein, respectively. “E” stands for Envelope protein, “RdRP” stands for “RNA dependent RNA-Polymerase”. Said term also includes fragments of a gene from SARS-CoV-2, e.g., RNA fragments of, e.g. 50, 75, 100, 150 or more nucleotides in length. Sometimes, when used herein, the term “gene” is omitted in the context of “SARS-CoV-2 E” or “SARS-CoV-2 RdRP”. Accordingly, the term “SARS-CoV-2 E” when used herein is equivalent to “SARS-CoV-2 E gene”, and vice versa, or the term “SARS-CoV-2 RdRP” when used herein is equivalent to “SARS-CoV-2 RdRP gene”, and vice versa.

Similarly, a “gene” from IBV (i.e., Influenza B virus) or IAV (i.e., influenza A virus) when used herein, e.g., in the context of IBV PA gene or IAV PB1 gene may refer to a nucleotide sequence, preferably RNA, such as IBV PA RNA or IAV PB1 RNA, which comprises an open reading frame (ORF) encoding IBV PA protein or IAV PB1 protein, respectively. “PB1” stands for RNA-directed RNA polymerase catalytic subunit, “PA” stands for “Polymerase acidic protein”. Said term also includes fragments of a gene from IBV (i.e., Influenza B virus) or IAV (i.e., influenza A virus), e.g., RNA fragments of, e.g. 50, 75, 100, 150 or more nucleotides in length. Sometimes, when used herein, the term “gene” is omitted in the context of “IBV PA” or “IAV PB1”. Accordingly, the term “IBV PA” when used herein is equivalent to “IBV PA gene”, and vice versa, or the term “IAV PB1” when used herein is equivalent to “IAV PB1 gene”, and vice versa.

A “gene” from human, e.g., RNase P gene when used herein is meant a nucleotide sequence, preferably RNA, such as human RNase P RNA, which comprises an open reading frame (ORF) encoding human RNase P protein. Said term also includes fragments of a gene from human RNase P, e.g., RNA fragments of, e.g., 50, 75, 100, 150 or more nucleotides in length. Sometimes, when used herein, the term “gene” is omitted in the context of “human RNase P”. Accordingly, the term “human RNase P” when used herein is equivalent to “human RNase P gene”, and vice versa.

As SARS-CoV-2 viruses are RNA viruses, the PCR-method according to the invention may comprise a reverse transcription step, at least one cycling step, which includes an amplifying step and a hybridizing step. Where the starting material for the PCR reaction is RNA, as in the case of SARS-CoV-2, complementary DNA (“cDNA”) is synthesized from RNA via reverse transcription. The resulting cDNA is then amplified using a PCR protocol, e.g., one as described above. Reverse transcriptases are known to those of ordinary skill in the art as enzymes found in retroviruses that can synthesize complementary single strands of DNA from an mRNA sequence as a template. A PCR used to amplify RNA products is referred to as reverse transcriptase PCR or “RT-PCR.” “RT-PCR” is a preferred PCR-method of the present invention.

The term “amplicon” as used herein may refer to nucleic acid that is a source and product of the PCR amplification.

Accordingly, the PCR-method of the invention may further comprise conducting an amplification step, preferably a simultaneous amplification step, with at least primer nucleotide sequences for a unique spike RNA. Preferably, the amplification step for a unique spike RNA is conducted simultaneously to the amplification step for SARS-CoV-2 RdRP gene, or the amplifications step for SARS-CoV-2 E gene, or the amplification step for human RNase P gene. Preferably, the amplification step for a unique spike RNA is conducted simultaneously to the amplification step for SARS-CoV-2 E gene and human RNase P gene or the amplifications step for SARS-CoV-2 RdRP gene and human RNase P gene. Preferably, the amplification step for a unique spike RNA is conducted simultaneously to the amplification step for SARS-CoV-2 RdRP gene, the amplifications step for SARS-CoV-2 E gene, and the amplification step for human RNase P gene. As explained herein, unique spike RNA serves preferably a control.

In particular, according to the PCR-methods of the present invention, for the amplification step of for SARS-CoV-2 RdRP gene at least the primer nucleotide sequences set forth in SEQ ID NOs: 1 and 2 are used. Furthermore, according to the PCR-methods of the present invention, for the amplification step of for SARS-CoV-2 E gene, at least the primer nucleotide sequences set forth in SEQ ID NOs: 4 or 22 and 5 are used. According to the PCR-methods of the present invention, for the amplification step of for human RNase P gene, at least the primer nucleotide sequences set forth in SEQ ID NOs: 7 and 8 are used. Further, according to the PCR-methods of the present invention, for the amplification step of the unique spike RNA, at least the primer nucleotide sequences set forth in SEQ ID NOs: 17 and 18 are used.

As mentioned, the present invention relates to a PCR-method comprising conducting an amplification step with at least the primer nucleotide sequences set forth in SEQ ID NOs: 1 and 2. In particular, primers of SEQ ID Nos 1 and 2 are used for the amplification of the SARS-CoV-2 RdRP gene. The inventors of the present invention designed and synthetized primers oligonucleotides suitable in PCR reactions for the amplification of nucleic acids derived from SARS-CoV-2 virus resulting in a PCR-method with higher specificity. In particular, the inventors surprisingly found that amplification of the SARS-CoV-2 RdRP gene using the combination of primers of SEQ ID Nos: 1 and 2 resulted in a PCR-method with higher specificity for the SARS-CoV-2 RdRP gene (see FIG. 1 and Table 1). In particular, the primer of SEQ ID NO: 1 is a forward primer that was generated by converting the degenerate nucleotides of the primer nucleotide sequence of SEQ ID NOs: 14 into non-degenerate, i.e., specific nucleotides, thereby obtaining the primer nucleotide sequence having SEQ ID NOs: 1. In this primer, mixed bases commonly used in order to amplify the SARS-CoV-2 RdRP gene were substituted with nucleotide “A”, based on consensus sequences of 700 known SARS-CoV-2 genomes. The primer of SEQ ID NO: 2 is a reverse primer for the amplification of the which the SARS-CoV-2 RdRP gene, which the present inventors designed and for which, when used together with the forward primer of SEQ ID NO: 1 they observed a significant improvement of the known and commonly applied PCR-method of Charité, Germany, see, e.g., Corman et al. (2020), cited above. The inventors surprisingly found that the combination of the above defined primers of SEQ ID NO: 1 and 2 led to a PCR-method that has better specificity for the SARS-CoV-2 nucleic acid detection.

The present invention also relates to a PCR-method further comprising conducting an amplification step with at least the primer nucleotide sequences set forth in SEQ ID NOs: 4 or 22 and 5. The primers of SEQ ID NOs: 4 or 22 and 5 are used for amplification of the SARS-CoV-2 E gene. Preferably, the PCR-method is a multiplex RT-PCR which comprises simultaneously conducting an amplification step with at least primers of SEQ ID Nos: 1 and 2, and at least primers of SEQ ID NOs: 7 and 8. The primers of SEQ ID Nos: 7 and 8 are used to amplify human RNase P.

Preferably, the PCR-method is a multiplex RT-PCR which comprises simultaneously conducting an amplification step with at least primers of SEQ ID Nos: 1 and 2, and at least primers of SEQ ID NO: 4 or 22 and 5 and at least primers of SEQ ID NOs: 7 and 8.

Furthermore, the PCR-method may further comprise conducting an amplification step with at least the primer nucleotide sequences set forth in SEQ ID Nos: 17 and 18, for the amplification of the unique spike RNA defined elsewhere herein.

Thus, it is also preferred that the PCR-method is a multiplex RT-PCR which comprises simultaneously conducting an amplification step with at least primers of SEQ ID Nos: 1 and 2, and at least primers of SEQ ID NOs: 17 and 18. It is also preferred that the PCR-method is a multiplex RT-PCR which comprises simultaneously conducting an amplification step with at least primers of SEQ ID Nos: 1 and 2, at least primers of SEQ ID Nos: 4 or 22 and 5 and at least primers of SEQ ID NOs: 17 and 18. It is also preferred that the PCR-method is a multiplex RT-PCR which comprises simultaneously conducting an amplification step with at least primers of SEQ ID Nos: 1 and 2, at least primers of SEQ ID Nos: 4 or 22 and 5, at least the primers of SEQ ID NOs: 7 and 8, and at least primers of SEQ ID NOs: 17 and 18.

The PCR-method of the invention further comprises a probe, as defined elsewhere herein, comprising the nucleotide sequence set forth in SEQ ID NO: 3 (for amplification of RdRP gene). In particular, the inventors generated the probe of SEQ ID NO: 3 by converting all degenerate nucleotides of the probe having SEQ ID NO: 16 into non-degenerate, i.e., specific nucleotides, thereby obtaining the probe having SEQ ID NO: 3. The PCR-method of the invention further comprises a probe, comprising the nucleotide sequence set forth in SEQ ID NO: 6 (for amplification of E gene). The PCR-method of the invention further comprises a probe, comprising the nucleotide sequence set forth in SEQ ID NO: 9 (for the amplification of human RNase P). The PCR-method of the invention further comprises a probe, comprising the nucleotide sequence set forth in SEQ ID NO: 19 (for the amplification of unique spike RNA). Examples of commonly used probes are TAQMAN® probes, Molecular Beacon probes, SCORPION® probes, and SYBR® Green probes. In the context of the present invention, the use of TaqMan® probes is preferred. TaqMan probes consist of a fluorophore covalently attached to the 5′-end of the oligonucleotide probe and a quencher at the 3′-end. Several different fluorophores (e.g. 6-carboxyfluorescein, acronym: FAM, or tetrachlorofluorescein, acronym: TET) and quenchers (e.g. tetramethylrhodamine, acronym: TAMRA or Black Hole Quencher® Dyes) are available. In particular, in the context of the present invention the probes of SEQ ID Nos: 3 and 6, are labeled with FAM fluorophores and the probes of SEQ ID Nos: 9 and 19 are labeled with HEX fluorophore.

Further, the PCR-method of the invention may further comprise a positive control, as defined elsewhere herein, in particular a ribonucleic acid having the sequence set forth in SEQ ID NO: 10 is used as positive control for the RdRP gene, a ribonucleic acid having the sequence set forth in SEQ ID NO: 11 or 23 is used as positive control for the E gene, a ribonucleic acid having the sequence set forth in SEQ ID NO: 12 is used as positive control for the human RNase P gene.

The present invention also relates to a kit, e.g., for detecting SARS-CoV-2 nucleic acid in a sample, defined elsewhere herein. The kit may comprise components necessary to carry out the methods of the present invention. Kits of the invention can include at least one pair of specific primers for the amplification of SARS-CoV-2 nucleic acid and at least one probe hybridizing specifically with the amplification products. Kits can include fluorophoric moieties for labeling the primers or probes or the primers and probes are already labeled with donor and corresponding acceptor fluorescent moieties. Kits can also include a package insert having instructions thereon for using the primers, probes, and fluorophoric moieties to detect the presence or absence of SARS-CoV-2 nucleic acid in a sample. In this context, the kit is comprising at least the primer nucleotide sequences set forth in SEQ ID NOs: 1 and 2, optionally a probe comprising the nucleotide sequence set forth in SEQ ID NO: 3 and optionally means for carrying out a PCR amplification step. Said means are known to the skilled person and can include standard reagents for PCR-methods like PCR buffer, DNA polymerase, Magnesium MgCl2 and water. The kit may further comprise at least the primer nucleotide sequences set forth in SEQ ID NOs: 7 and 8, and optionally a probe comprising the nucleotide sequence set forth in SEQ ID NO: 9. Further, the kit may comprise at least the primer nucleotide sequences set forth in SEQ ID NOs: 17 and 18, and optionally a probe comprising the nucleotide sequence set forth in SEQ ID NO: 19. Finally, the kit may further comprise a ribonucleic acid having the sequence set forth in SEQ ID NO: 10, and SEQ ID NO: 11 or 23, SEQ ID NO: 12 and SEQ ID NO: 20.

Another aspect of the invention is the use of the primer nucleotide sequences set forth in SEQ ID NOs: 1 and 2 for the

(i) in vitro detection of SARS-CoV-2 in a sample,

(ii) in vitro detection of an infection of a subject with SARS-CoV-2,

(iii) in vitro detection of a contamination of a blood sample with SARS-CoV-2, or

(iv) monitoring the therapy of SARS-CoV-2 in vitro.

Accordingly, a probe comprising the nucleotide sequence set forth in SEQ ID NO: 3 is used for the in vitro detection, together with the primers set forth in SEQ ID NOs: 1 and 2.

As used herein, the term “in vitro detection” refers to the detection outside, i.e., ex vivo of a mammalian subject, e.g., via the PCR-methods as defined herein. As used herein “monitoring the therapy of SARS-CoV-2 in vitro” refers to a companion diagnostic accompanying a therapy for the treatment of SARS-CoV-2 infection. For example, a sample from patient who is subject to such a therapy may be controlled for the presence or absence of SARS-CoV-2 which may be indicative of an effect of the therapy.

In some aspects/embodiments, the present invention relates to a PCR-method comprising conducting a simultaneous amplification step with at least primer nucleotide sequences for SARS-CoV-2 RdRP, at least primer nucleotide sequences for SARS-CoV-2 E gene and at least primer nucleotide sequences for human RNase P, wherein said PCR-method is a multiplex PCR, preferably a multiplex real-time RT-PCR, further preferably said multiplex real-time RT-PCR method comprising conducting said simultaneous amplification step with at least one (e.g., two different) probe specific for the SARS-CoV-2 RdRP amplicon, at least one (e.g., two different) probe specific for the SARS-CoV-2 E amplicon and at least one (e.g., two different) probe specific for the human RNase P amplicon.

In some aspects/embodiments, the present invention relates to a PCR-method comprising: (i) a first PCR-method comprising conducting a simultaneous amplification step with at least primer nucleotide sequences for SARS-CoV-2 RdRP and at least primer nucleotide sequences for human RNase P, preferably with at least one (e.g., two different) probe specific for the SARS-CoV-2 RdRP amplicon and at least one (e.g., two different) probe specific for human RNase P amplicon; and/or (ii) a second PCR-method comprising conducting a simultaneous amplification step with at least primer nucleotide sequences for SARS-CoV-2 E gene and at least primer nucleotide sequences for human RNase P, preferably with at least one (e.g., two different) probe specific for the SARS-CoV-2 E amplicon and at least one (e.g., two different) probe specific for the human RNase P amplicon, wherein the same source material suspected to comprise SARS-CoV-2 nucleic acids is used for the PCR of (i) and (ii), wherein said first and second PCR-methods are multiplex PCR methods, preferably multiplex real-time RT-PCR methods.

In some aspects/embodiments, the present invention relates to a PCR-method comprising: (i) a first PCR-method comprising conducting a simultaneous amplification step with at least primer nucleotide sequences for SARS-CoV2 E gene and at least primer nucleotide sequences for a unique spike RNA, preferably with at least one (e.g., two different) probe specific for the SARS-CoV-2 E amplicon and at least one (e.g., two different) probe specific for the unique spike RNA amplicon; (ii) a second PCR-method comprising conducting a simultaneous amplification step with at least primer nucleotide sequences for SARS-CoV-2 RdRP gene and at least primer nucleotide sequences for human RNase P, preferably with at least one (e.g., two different) probe specific for the SARS-CoV-2 RdRP amplicon and at least one (e.g., two different) probe specific for the human RNase P amplicon; and (iii) a third PCR-method comprising conducting a simultaneous amplification step with at least primer nucleotide sequences for SARS-CoV-2 RdRP gene, at least primer nucleotide sequences for SARS-CoV-2 E gene, at least primer nucleotide sequences for human RNase P and at least primer nucleotide sequences for a unique spike RNA, preferably with at least one (e.g., two different) probe specific for the SARS-CoV-2 RdRP amplicon, at least one (e.g., two different) probe specific for the SARS-CoV-2 E amplicon and at least one probe specific for the human RNase P amplicon and at least one probe specific for the unique spike RNA amplicon; wherein the same source material suspected to comprise SARS-CoV-2 nucleic acids is used for the PCR-method of (i) and (ii), and wherein the PCR-method of (iii) further comprises as positive control a ribonucleic acid for SARS-CoV-2 RdRP gene, a ribonucleic acid for SARS-CoV-2 E gene, a ribonucleic acid for human RNase P and a ribonucleic acid for unique spike RNA, wherein said first, second and third PCR-methods are multiplex PCR methods, preferably multiplex real-time RT-PCR methods.

In some aspects/embodiments, the PCR method, kit or use of the present invention, comprising conducting a simultaneous amplification step with at least primer nucleotide sequences for SARS-CoV-2 RdRP, at least primer nucleotide sequences for SARS-CoV-2 E gene and at least primer nucleotide sequences for human RNase P, wherein said PCR-method is a multiplex PCR.

In some aspects/embodiments of the PCR method, kit or use of the present invention, said simultaneous amplification step (e.g., one or more) is further conducted with at least primer nucleotide sequences for RNA-directed RNA polymerase catalytic subunit protein of Influenza virus A (i.e. PB1 IAV) and with at least primer nucleotide sequences for Polymerase acidic protein of Influenza virus B (i.e., PA IBV), preferably said PCR-method is a multiplex PCR method, further preferably said multiplex PCR method is a multiplex real-time RT-PCR method, most preferably said multiplex real-time RT-PCR method comprising conducting said simultaneous amplification step with at least one (e.g., two different) probe specific for said PB1 IAV amplicon and at least one (e.g., two different) probe specific for PA IBV amplicon.

In some aspects/embodiments of the PCR method, kit or use of the present invention, for the amplification step for PB1 of IAV at least the primer nucleotide sequences set forth in SEQ ID NOs: 83-84 or 121-128 are used, preferably SEQ ID NOs: 83-84; further preferably with at least one probe selected from the group consisting of: SEQ ID NOs: 85, 129-131, preferably SEQ ID NO: 85.

In some aspects/embodiments of the PCR method, kit or use of the present invention, for the amplification step for PA IBV at least the primer nucleotide sequences set forth in SEQ ID NOs: 86-87 or 132-144 are used, preferably SEQ ID NOs: 86-87; further preferably with at least one probe selected from the group consisting of: SEQ ID NOs: 88, 145-153, preferably SEQ ID NO: 88.

In some aspects/embodiments of the PCR method, kit or use of the present invention, for the amplification step for SARS-CoV-2 RdRP at least the primer nucleotide sequences set forth in SEQ ID NOs: 1 and 2, SEQ ID NOs: 27 and 28, SEQ ID NOs: 33 and 34, SEQ ID NOs: 43 and 44, SEQ ID NOs: 54 and 55, SEQ ID NOs: 65 and 66, SEQ ID NOs: 76 and 77, SEQ ID NOs: 109 and 110, SEQ ID NOs: 109 and 111 or SEQ ID NOs: 109 and 112 are used, preferably with at least one (e.g., two) probe selected from the group consisting of: SEQ ID NOs: 29, 35, 45, 46, 56, 57, 67, 68, 78, 79 and 113-120, further preferably with two partially overlapping (e.g., partially comprising the identical sequence, but non-identical) probes selected from the group consisting of: SEQ ID NOs: 45-46, SEQ ID NOs: 56-57, 67-68, 78-79.

In some aspects/embodiments of the PCR method, kit or use of the present invention, for the amplification step for SARS-CoV-2 E gene at least the primer nucleotide sequences set forth in SEQ ID NOs: 4 or 22 and 5, SEQ ID NOs: 24-25, SEQ ID NOs: 30-31, SEQ ID NOs: 39-40, 50-51, 61-62, 72-73, or any one of 89-101 with 102 are used, preferably with at least one (e.g., two) probe selected from the group consisting of: SEQ ID NOs: 26, 32, 41, 42, 52, 53, 63, 64, 74, 75, 103-108, further preferably with two probes selected from the group consisting of: SEQ ID NOs: 41-42, SEQ ID NOs: 52-53, 63-64, 74-75.

In some aspects/embodiments of the PCR method, kit or use of the present invention, for the amplification step for human RNase P at least the primer nucleotide sequences set forth in SEQ ID NOs: 7 and 8, or SEQ ID NOs: 36-37, 47-48, 58-59, 69-70, 80-81 or 154-155 are used, preferably with at least one probe selected from the group consisting of: SEQ ID NOs: 38, 49, 60, 71, 82 or 156.

In some aspects/embodiments of the present invention, the PCR method, kit or use comprising: (i) a probe comprising the nucleotide sequence set forth in SEQ ID NO: 3, a probe comprising the nucleotide sequence set forth in SEQ ID NO: 6, a probe comprising the nucleotide sequence set forth in SEQ ID NO: 9, a probe comprising the nucleotide sequence set forth in SEQ ID NO: 19; and/or (ii) a probe comprising the nucleotide sequence set forth in SEQ ID NO: 26, a probe comprising the nucleotide sequence set forth in SEQ ID NO: 29, preferably in combination with primer nucleotide sequences set forth in SEQ ID NOs: 24-25 and 27-28; and/or (iii) a probe comprising the nucleotide sequence set forth in SEQ ID NO: 32, a probe comprising the nucleotide sequence set forth in SEQ ID NO: 35, a probe comprising the nucleotide sequence set forth in SEQ ID NO: 38, preferably in combination with primer nucleotide sequences set forth in SEQ ID NOs: 30-31, 33-34 and 36-37; and/or (iv) a probe comprising the nucleotide sequence set forth in SEQ ID NO: 41, a probe comprising the nucleotide sequence set forth in SEQ ID NO: 42, a probe comprising the nucleotide sequence set forth in SEQ ID NO: 45, a probe comprising the nucleotide sequence set forth in SEQ ID NO: 46, a probe comprising the nucleotide sequence set forth in SEQ ID NO: 49, preferably in combination with primer nucleotide sequences set forth in SEQ ID NOs: 39-40, 43-44, 47-48; and/or (v) a probe comprising the nucleotide sequence set forth in SEQ ID NO: 52, a probe comprising the nucleotide sequence set forth in SEQ ID NO: 53, a probe comprising the nucleotide sequence set forth in SEQ ID NO: 56, a probe comprising the nucleotide sequence set forth in SEQ ID NO: 57, a probe comprising the nucleotide sequence set forth in SEQ ID NO: 60, preferably in combination with primer nucleotide sequences set forth in SEQ ID NOs: 50-51, 54-55, 58-59; and/or (vi) a probe comprising the nucleotide sequence set forth in SEQ ID NO: 63, a probe comprising the nucleotide sequence set forth in SEQ ID NO: 64, a probe comprising the nucleotide sequence set forth in SEQ ID NO: 67, a probe comprising the nucleotide sequence set forth in SEQ ID NO: 68, a probe comprising the nucleotide sequence set forth in SEQ ID NO: 71, preferably in combination with primer nucleotide sequences set forth in SEQ ID NOs: 61-62, 65-66 and 69-70; and/or (vii) a probe comprising the nucleotide sequence set forth in SEQ ID NO: 74, a probe comprising the nucleotide sequence set forth in SEQ ID NO: 75, a probe comprising the nucleotide sequence set forth in SEQ ID NO: 78, a probe comprising the nucleotide sequence set forth in SEQ ID NO: 79, a probe comprising the nucleotide sequence set forth in SEQ ID NO: 82, a probe comprising the nucleotide sequence set forth in SEQ ID NO: 85, a probe comprising the nucleotide sequence set forth in SEQ ID NO: 88, preferably in combination with primer nucleotide sequences set forth in SEQ ID NOs: 72-73, 76-77, 80-81, 83-84 and 86-87.

In some aspects/embodiments of the present invention, the PCR method, kit or use comprising at least the primer nucleotide sequences set forth in SEQ ID NOs: 1 and 2 or SEQ ID NOs: 27-28, SEQ ID NOs: 33-34, SEQ ID NOs: 43-44, SEQ ID NOs: 54-55, SEQ ID NOs: 65-66, SEQ ID NOs: 76-77, SEQ ID NOs: 109-110, SEQ ID NOs: 109 and 111 or SEQ ID NOs: 109 and 112, optionally at least one (e.g., two different) probe comprising the nucleotide sequence selected from the group consisting of: SEQ ID NO: 3, SEQ ID NOs: 29, 35, 45, 46, 56, 57, 67, 68, 78, 79 or 113-120, preferably comprising two (e.g., partially overlapping, but non-identical) probes selected from the group consisting of: SEQ ID NOs:

45-46, SEQ ID NOs: 56-57, 67-68, 78-79 and further optionally means for carrying out a PCR amplification step, preferably said kit is a multiplex PCR kit, preferably said multiplex PCR kit is a multiplex real-time RT-PCR kit.

In some aspects/embodiments of the present invention, the PCR method, kit or use comprising at least the primer nucleotide sequences set forth in SEQ ID NOs: 4 or 22 and 5 or SEQ ID NOs: 24-25, SEQ ID NOs: 30-31, SEQ ID NOs: 39-40, 50-51, 61-62, 72-73 or any one of 89-101 in combination with 102, and optionally at least one (e.g., two different) probe comprising the nucleotide sequence selected from the group consisting of: SEQ ID NO: 6, SEQ ID NOs: 26, 32, 41, 42, 52, 53, 63, 64, 74, 75, 103-108, preferably comprising two (e.g., different) probes selected from the group consisting of: SEQ ID NOs: 41-42, SEQ ID NOs: 52-53, 63-64, 74-75.

In some aspects/embodiments of the present invention, the PCR method, kit or use comprising at least the primer nucleotide sequences set forth in SEQ ID NOs: 7 and 8 or SEQ ID NOs: 36-37, 47-48, 58-59, 69-70, 80-81 or 154-155, and optionally at least one (e.g., two, e.g., two different) probe comprising the nucleotide sequence selected from the group consisting of: SEQ ID NO: 9, SEQ ID NOs: 38, 49, 60, 71, 82 or 156.

In some aspects/embodiments of the present invention, the PCR method, kit or use comprising at least the primer nucleotide sequences set forth in SEQ ID NOs: 83-84 or 121-128, preferably in SEQ ID NOs: 83-84, optionally at least one (e.g., two, e.g., two different) probe selected from the group consisting of: SEQ ID NOs: 85, 129-131, further preferably SEQ ID NO: 85.

In some aspects/embodiments of the present invention, the PCR method, kit or use comprising at least the primer nucleotide sequences set forth in SEQ ID NOs: 86-87 or 132-144 are used, preferably SEQ ID NOs: 86-87, optionally at least one (e.g., two, e.g., two different) probe selected from the group consisting of: SEQ ID NOs: 88, 145-153, further preferably SEQ ID NO: 88.

In some aspects/embodiments of the present invention, the PCR method, kit or use comprising at least the primer nucleotide sequences set forth in SEQ ID NOs: 86-87 or 132-144 are used, preferably SEQ ID NOs: 86-87, optionally at least one (e.g., two, e.g., two different) probe selected from the group consisting of: SEQ ID NOs: 88, 145-153, further preferably SEQ ID NO: 88.

In some aspects/embodiments of the present invention, the PCR method, kit or use comprising conducting a simultaneous (e.g. multiplexed) amplification step with at least primer nucleotide sequences targeting the spike (S) gene of SARS-CoV-2, preferably with primers having SEQ ID NOs: 157-158, preferably with a probe/s having SEQ ID NO: 161 or 162.

In some aspects/embodiments of the present invention, the PCR method, kit or use comprising conducting a simultaneous (e.g. multiplexed) amplification step with at least primer nucleotide sequences targeting two mutations (deletions) that are present in the B.1.1.7 variant/mutant of SARS-CoV-2, preferably with primers having SEQ ID NOs: 159-160, preferably with a probe/s having SEQ ID NO: 161 or 162.

In some aspects/embodiments of the PCR method, kit or use of the present invention, said primer and/or probe nucleotide sequence/s comprising one or more (e.g., 2, 3 or 4) Locked Nucleic Acids (LNA)-modified nucleotides (e.g., LNA is a synthetic nucleic acid analogue containing a bridged, bicyclic sugar moiety, e.g., a methylene linkage between the 2′ oxygen and the 4′ carbon of the ribose ring), preferably said one or more (e.g., 2, 3 or 4) LNA-modified nucleotides are LNA-modified thymine residues (e.g., LNA-T) and/or LNA-modified adenosine residues (e.g., LNA-A).

In some aspects/embodiments, the present invention relates to use of the primer nucleotide sequences, wherein said primer sequences: (a) set forth in SEQ ID NOs: 1-2 or SEQ ID NOs: 27-28, SEQ ID NOs: 33-34, SEQ ID NOs: 43-44, SEQ ID NOs: 54-55, SEQ ID NOs: 65-66, SEQ ID NOs: 76-77, SEQ ID NOs: 109-110, SEQ ID NOs: 109 and 111 SEQ ID NOs: 109 and 112, preferably with at least one (e.g., two) probe selected from the group consisting of: SEQ ID NOs: 29, 35, 45, 46, 56, 57, 67, 68, 78, 79 and 113-120, further preferably with two partially overlapping probes (e.g., non-identical probes) selected from the group consisting of: SEQ ID NOs: 45-46, SEQ ID NOs: 56-57, 67-68, 78-79; and/or (b) set forth in SEQ ID NOs: 4 or 22 and 5, or SEQ ID NOs: 24-25, SEQ ID NOs: 30-31, SEQ ID NOs: 39-40, 50-51, 61-62, 72-73, any one of 89-101 with 102, preferably with at least one (e.g., two) probe selected from the group consisting of: SEQ ID NOs: 26, 32, 41, 42, 52, 53, 63, 64, 74, 75, 103-108, further preferably with two probes selected from: SEQ ID NOs: 41-42, SEQ ID NOs: 52-53, 63-64, 74-75; and/or (c) set forth in SEQ ID NOs: 83-84 or 121-128, preferably SEQ ID NOs: 83-84, further preferably with at least one probe selected from the group consisting of: SEQ ID NOs: 85, 129-131, most preferably SEQ ID NO: 85; and/or (d) set forth in SEQ ID NOs: 86-87 or 132-144, preferably SEQ ID NOs: 86-87, further preferably with at least one probe selected from the group consisting of: SEQ ID NOs: SEQ ID NOs: 88, 145-153, preferably SEQ ID NO: 88; wherein said sequences of (a), (b), (c) and (d) are used alone or in combination with one another for one or more of the following: (i) in vitro detection (e.g., simultaneous, e.g., multiplexed detection) of SARS-CoV-2 and/or IAV and/or IBV in a sample, (ii) in vitro detection (e.g., simultaneous, e.g., multiplexed detection) of an infection of a subject with SARS-CoV-2 and/or IAV and/or IBV, (iii) in vitro detection (e.g., simultaneous, e.g., multiplexed detection) of a contamination of a blood sample with SARS-CoV-2 and/or IAV and/or IBV, or (iv) monitoring (e.g., simultaneous, e.g., multiplexed monitoring) the therapy of SARS-CoV-2 and/or IAV and/or IBV in vitro, wherein said use is the use for multiplex PCR detection, preferably a multiplex real-time RT-PCR detection; (v) for/in the PCR-method according of any one of the preceding claims, preferably said method is an in vitro or ex vivo method.

In some aspects/embodiments of the PCR method, kit or use of the present invention is an in vitro or ex vivo PCR method, kit or use.

In some aspects/embodiments of the present invention, the PCR method, kit or use of the present invention relates to dual probes (e.g., two probes used in combination, e.g., partially overlapping, non-identical probes) capable of increasing both sensitivity and specificity of the PCR method, kit or use.

In some aspects/embodiments, the PCR method, kit or use of the present invention can be a useful diagnostic tool to differentiate SARS-CoV-2 from the most common seasonal influenzas.

In some aspects/embodiments, the PCR method, kit or use of the present invention are room temperature-stable for up to one month, providing one of the few RT-qPCR tests that eliminates the need for cold chain shipping and storage.

In some aspects/embodiments, the PCR method, kit or use of the present invention relate to suitable one-step RT-qPCR condition disclosed in Example 2 herein.

In some aspects/embodiments, the PCR method, kit or use of the present invention relate to positive controls as described in Example 2 herein.

In some aspects/embodiments, the PCR method, kit or use of the present invention relate Table 2, which discloses embodiments of the present invention.

In some aspects/embodiments, the PCR method, kit or use of the present invention relate Table 3, which discloses embodiments of the present invention.

In some aspects/embodiments, the PCR method, kit or use of the present invention relate Table 4, which discloses embodiments of the present invention.

In some aspects/embodiments, the PCR method, kit or use of the present invention relate Table 5, which discloses embodiments of the present invention.

In some aspects/embodiments, the PCR method, kit or use of the present invention overcomes uneven amplification/folding, false negatives, poor sensitivity and specificity and/or preferential amplification of certain specific targets, primer dimers etc. as well as reduction of false-positive and false-negative read outs known to be associated with multiplexing PCR-methods, kits or uses.

In some aspects/embodiments, the PCR method, kit or use of the present invention comprise positive control/s spiked with either baker's yeast tRNA or salmon sperm DNA (ssDNA) or both, e.g., to act as carriers and decoys for nucleases and then lyophilized with the positive control.

In some aspects/embodiments of the present invention, the probe of the present invention is a hydrolysis probe, e.g., dually labelled probes.

In some aspects/embodiments, dual-versus single-probe reactions increased sensitivity and dynamic range of the methods and kits of the present invention.

In some aspects/embodiments, the PCR method, kit or use of the present invention incorporate varying numbers of LNA nucleotides (e.g., 1, 2, 3, 4, 5, etc.) enabling the design of shorter primers (e.g., forward or reverse) that do not overlap with the original Charité forward primer, while maintaining sufficient melting temperatures.

In some aspects/embodiments, LNA nucleotides can be synthesized as disclosed by Madsen et al., 2010 (Org Biomol Chem. 2010 Nov. 7; 8(21):5012-6).

In some aspect/embodiments the present invention relates to a PCR-method comprising: (i) a first PCR method comprising conducting a simultaneous (multiplexed) amplification step with at least primer nucleotide sequences for SARS-CoV2 E gene disclosed herein (optionally with probe/s disclosed herein) and at least primer nucleotide sequences for IAV PB1 gene disclosed herein (optionally with probe/s disclosed herein) and at least primer nucleotide sequences for RNase P (optionally with probe/s disclosed herein); (ii) a second PCR-method comprising conducting a simultaneous (multiplexed) amplification step with at least primer nucleotide sequences for SARS-CoV-2 RdRP gene disclosed herein (optionally with probe/s disclosed herein) and at least primer nucleotide sequences for IBV PA gene disclosed herein (optionally with probe/s disclosed herein) and at least primer nucleotide sequences for RNase P (optionally with probe/s disclosed herein). Advantageously, said method allowing differentiation of SARS-CoV-2, IAV, and IBV.

In some aspect/embodiments the present invention relates to a PCR-method comprising conducting a simultaneous (multiplexed) amplification step with at least primer and probe nucleotide sequences for SARS-CoV2 E gene and SARS-CoV-2 RdRP gene disclosed herein (both labelled with FAM), IAV PB1 and IBV PA genes (both labelled with YY), and RNase P (Cy5). This allows for the differentiation of SARS-CoV-2 and influenza (but not the distinction between IAV and IBV).

### ITEMS OF THE INVENTION

The present invention may also be summarized by the following items:
1. A PCR-method comprising conducting a simultaneous amplification step with at least primer nucleotide sequences for SARS-CoV-2 RdRP, at least primer nucleotide sequences for SARS-CoV-2 E gene and at least primer nucleotide sequences for human RNase P, preferably said PCR-method is a multiplex PCR method, further preferably said multiplex PCR method is a multiplex real-time RT-PCR method, most preferably said multiplex real-time RT-PCR method comprising conducting said simultaneous amplification step in the presence of/with at least one (e.g., two, e.g., two partially overlapping, but non-identical probes) dually-labeled (e.g., with Dye/Fluorophore and/or Quencher) nucleotide sequence (e.g., DNA) hydrolysis probe specific (e.g., complementary) for SARS-CoV-2 RdRP amplicon, at least one (e.g., two, e.g., two overlapping probes) dually-labeled (e.g., with Dye/Fluorophore and/or Quencher) nucleotide sequence (e.g., DNA) hydrolysis probe specific (e.g., complementary) for SARS-CoV-2 E amplicon and at least one dually-labeled (e.g., with Dye/Fluorophore and Quencher) nucleotide sequence (e.g., DNA) hydrolysis probe specific (e.g., complementary) for human RNase P amplicon.
2. A PCR-method comprising
   (i) a first PCR-method comprising conducting a simultaneous amplification step with at least primer nucleotide sequences for SARS-CoV-2 RdRP and at least primer nucleotide sequences for human RNase P; preferably in the presence of/with at least one (e.g., two, e.g., two partially overlapping probes, but non-identical) dually-labeled (e.g., with Dye/Fluorophore and/or Quencher) nucleotide sequence (e.g., DNA) hydrolysis probe specific (e.g., complementary) for SARS-CoV-2 RdRP amplicon and at least one dually-labeled (e.g., with Dye/Fluorophore and Quencher) nucleotide sequence (e.g., DNA) hydrolysis probe specific (e.g., complementary) for human RNase P amplicon and/or
   (ii) a second PCR-method comprising conducting a simultaneous amplification step with at least primer nucleotide sequences for SARS-CoV-2 E gene and at least primer nucleotide sequences for human RNase P, preferably in the presence of/with at least one (e.g., two) dually-labeled (e.g., with Dye/Fluorophore and/or Quencher) nucleotide sequence (e.g., DNA) hydrolysis probe specific (e.g., complementary) for SARS-CoV-2 E amplicon and at least one dually-labeled (e.g., with Dye/Fluorophore and Quencher) nucleotide sequence (e.g., DNA) hydrolysis probe specific (e.g., complementary) for human RNase P amplicon;
   wherein the same source material suspected to comprise SARS-CoV-2 nucleic acids is used for the PCR of (i) and (ii), preferably wherein said first and second PCR-methods are multiplex PCR methods, further preferably said first and second multiplex PCR methods are multiplex real-time RT-PCR methods.
3. A PCR-method comprising
   (i) a first PCR-method comprising conducting a simultaneous amplification step with at least primer nucleotide sequences for SARS-CoV2 E gene and at least primer nucleotide sequences for a unique spike RNA, preferably in the presence of/with at least one (e.g., two) dually-labeled (e.g., with Dye/Fluorophore and/or Quencher) nucleotide sequence (e.g., DNA) hydrolysis probe specific (e.g., complementary) for SARS-CoV-2 E amplicon and at least one (e.g., two) dually-labeled (e.g., with Dye/Fluorophore and Quencher) nucleotide sequence (e.g., DNA) hydrolysis probe specific (e.g., complementary) for said unique spike RNA;
   (ii) a second PCR-method comprising conducting a simultaneous amplification step with at least primer nucleotide sequences for SARS-CoV-2 RdRP gene and at least primer nucleotide sequences for human RNase P, preferably in the presence of/with at least one (e.g., two, e.g., two partially overlapping probes, but non-identical) dually-labeled (e.g., with Dye/Fluorophore and/or Quencher) nucleotide sequence (e.g., DNA) hydrolysis probe specific (e.g., complementary) for SARS-CoV-2 RdRP amplicon and at least one dually-labeled (e.g., with Dye/Fluorophore and Quencher) nucleotide sequence (e.g., DNA) hydrolysis probe specific (e.g., complementary) for human RNase P amplicon; and
   (iii) a third PCR-method comprising conducting a simultaneous amplification step with at least primer nucleotide sequences for SARS-CoV-2 RdRP gene, at least primer nucleotide sequences for SARS-CoV-2 E gene, at least primer nucleotide sequences for human RNase P and at least primer nucleotide sequences for a unique spike RNA, preferably in the presence of/with at least one (e.g., two, e.g., two overlapping probes) dually-labeled (e.g., with Dye/Fluorophore and/or Quencher) nucleotide sequence (e.g., DNA) hydrolysis probe specific (e.g., complementary) for SARS-CoV-2 RdRP amplicon, at least one (e.g., two) dually-labeled (e.g., with Dye/Fluorophore and/or Quencher) nucleotide sequence (e.g., DNA) hydrolysis probe specific (e.g., complementary) for SARS-CoV-2 E amplicon and at least one dually-labeled (e.g., with Dye/Fluorophore and Quencher) nucleotide sequence (e.g., DNA) hydrolysis probe specific (e.g., complementary) for human RNase P amplicon and at least one dually-labeled (e.g., with Dye/Fluorophore and Quencher) nucleotide sequence (e.g., DNA) hydrolysis probe specific (e.g., complementary) for said unique spike RNA amplicon;
   wherein the same source material suspected to comprise SARS-CoV-2 nucleic acids is used for the PCR-method of (i) and (ii), and
   wherein the PCR-method of (iii) further comprises as positive control a ribonucleic acid for SARS-CoV-2 RdRP gene, a ribonucleic acid for SARS-CoV-2 E gene, a ribonucleic acid for human RNase P and a ribonucleic acid for unique spike RNA, preferably wherein said first, second and third PCR-methods are multiplex PCR methods, further preferably said multiplex PCR methods are multiplex real-time RT-PCR methods.
4. A PCR-method comprising
   (i) a first PCR-method comprising conducting a simultaneous (multiplexed) amplification step with at least primer nucleotide sequences for SARS-CoV2 E gene, at least primer nucleotide sequences for IAV PB1 gene and at least primer nucleotide sequences for human RNase P, preferably in the presence of/with at least one (e.g., two) dually-labeled (e.g., with Dye/Fluorophore and/or Quencher) nucleotide sequence (e.g., DNA) hydrolysis probe specific (e.g., complementary) for SARS-CoV-2 E amplicon, with of at least one (e.g., two) dually-labeled (e.g., with Dye/Fluorophore and/or Quencher) nucleotide sequence (e.g., DNA) hydrolysis probe specific (e.g., complementary) for IAV PB1 amplicon and at least one dually-labeled (e.g., with Dye/Fluorophore and Quencher) nucleotide sequence (e.g., DNA) hydrolysis probe specific (e.g., complementary) for human RNase P amplicon;
   (ii) a second PCR-method comprising conducting a simultaneous (multiplexed) amplification step with at least primer nucleotide sequences for SARS-CoV-2 RdRP gene and at least primer nucleotide sequences for human RNase P and with at least primer nucleotide sequences for IBV PA, preferably in the presence of/with at least one (e.g., two, e.g., two partially overlapping probes, but non-identical) dually-labeled (e.g., with Dye/Fluorophore and/or Quencher) nucleotide sequence (e.g., DNA) hydrolysis probe specific (e.g., complementary) for SARS-CoV-2 RdRP amplicon and at least one dually-labeled (e.g., with Dye/Fluorophore and Quencher) nucleotide sequence (e.g., DNA) hydrolysis probe specific (e.g., complementary) for human RNase P amplicon, and at least one dually-labeled (e.g., with Dye/Fluorophore and Quencher) nucleotide sequence (e.g., DNA) hydrolysis probe specific (e.g., complementary) for IBV PA amplicon.
5. A PCR-method comprising conducting a simultaneous amplification step with at least primer nucleotide sequences for SARS-CoV-2 RdRP, at least primer nucleotide sequences for SARS-CoV-2 E gene and at least primer nucleotide sequences for human RNase P, at least primer nucleotide sequences for IBV PA and at least primer nucleotide sequences for IAV PB1, preferably in the presence of/with at least one (e.g., two) dually-labeled (e.g., with Dye/Fluorophore and/or Quencher) nucleotide sequence (e.g., DNA) hydrolysis probe specific (e.g., complementary) for SARS-CoV-2 E amplicon, with of at least one (e.g., two) dually-labeled (e.g., with Dye/Fluorophore and/or Quencher) nucleotide sequence (e.g., DNA) hydrolysis probe specific (e.g., complementary) for IAV PB1 amplicon and at least one dually-labeled (e.g., with Dye/Fluorophore and Quencher) nucleotide sequence (e.g., DNA) hydrolysis probe specific (e.g., complementary) for human RNase P amplicon, at least one (e.g., two, e.g., two partially overlapping probes, but non-identical) dually-labeled (e.g., with Dye/Fluorophore and/or Quencher) nucleotide sequence (e.g., DNA) hydrolysis probe specific (e.g., complementary) for SARS-CoV-2 RdRP amplicon and at least one dually-labeled (e.g., with Dye/Fluorophore and Quencher) nucleotide sequence (e.g., DNA) hydrolysis probe specific (e.g., complementary) for IBV PA amplicon.
6. The PCR-method of any one of the preceding items, further comprising conducting said simultaneous amplification step (one or more) with at least primer nucleotide sequences for RNA-directed RNA polymerase catalytic subunit protein of Influenza virus A (IAV) (i.e. PB1, e.g., having UniProtKB—Q7TGB8, e.g., GenBank: AJ564806.1) and with at least primer nucleotide sequences for Polymerase acidic protein of Influenza virus B (IBV) (i.e., PA, e.g., UniProtKB—Q9QLI4, e.g., GenBank: AF102023.1), preferably said PCR-method is a multiplex PCR method, further preferably said multiplex PCR method is a multiplex real-time RT-PCR method, most preferably said multiplex real-time RT-PCR method comprising conducting said simultaneous amplification step in the presence of/with at least one (e.g., two) dually-labeled (e.g., with Dye/Fluorophore and Quencher) nucleotide sequence (e.g., DNA) hydrolysis probe specific (e.g., complementary) for said PB1 amplicon of IAV and at least one (e.g., two) dually-labeled (e.g., with Dye/Fluorophore and Quencher) nucleotide sequence (e.g., DNA) hydrolysis probe specific (e.g., complementary) for PA amplicon of IBV.
7. The PCR-method of any one of the preceding items, further comprising conducting an amplification step, preferably a simultaneous amplification step, with at least primer nucleotide sequences for a unique spike RNA, preferably wherein said PCR-method is a multiplex PCR, further preferably said multiplex PCR is a multiplex real-time RT-PCR method.
8. The PCR-method of any one of the preceding items, wherein for the amplification step for SARS-CoV-2 RdRP at least the primer nucleotide sequences set forth in SEQ ID NOs: 1 and 2, SEQ ID NOs: 27 and 28, SEQ ID NOs: 33 and 34, SEQ ID NOs: 43 and 44, SEQ ID NOs: 54 and 55, SEQ ID NOs: 65 and 66, SEQ ID NOs: 76 and 77, SEQ ID NOs: 109 and 110, SEQ ID NOs: 109 and 111 or SEQ ID NOs: 109 and 112 are used.
9. The PCR-method of any one of the preceding items, wherein for the amplification step for SARS-CoV-2 RdRP at least the primer nucleotide sequences set forth in SEQ ID NOs: 1 and 2, SEQ ID NOs: 27 and 28, SEQ ID NOs: 33 and 34, SEQ ID NOs: 43 and 44, SEQ ID NOs: 54 and 55, SEQ ID NOs: 65 and 66, SEQ ID NOs: 76 and 77, SEQ ID NOs: 109 and 110, SEQ ID NOs: 109 and 111 or SEQ ID NOs: 109 and 112 are used in the presence of at least one hydrolysis probe selected from the group consisting of: SEQ ID NOs: 29, 35, 45, 46, 56, 57, 67, 68, 78, 79 and 113-120, preferably in the presence of two (e.g., overlapping) hydrolysis probes selected from the group consisting of: SEQ ID NOs: 45-46, SEQ ID NOs: 56-57, 67-68, 78-79.
10. The PCR-method of any one of the preceding items, wherein for the amplification step for SARS-CoV-2 E gene at least the primer nucleotide sequences set forth in SEQ ID NOs: 4 or 22 and 5, SEQ ID NOs: 24-25, SEQ ID NOs: 30-31, SEQ ID NOs: 39-40, 50-51, 61-62, 72-73, any one of 89-101 with 102 are used.
11. The PCR-method of any one of the preceding items, wherein for the amplification step for SARS-CoV-2 E gene at least the primer nucleotide sequences set forth in SEQ ID NOs: 4 or 22 and 5, SEQ ID NOs: 24-25, SEQ ID NOs: 30-31, SEQ ID NOs: 39-40, 50-51, 61-62, 72-73, any one of 89-101 with 102 are used in the presence of at least one hydrolysis probe selected from the group consisting of: SEQ ID NOs: 26, 32, 41, 42, 52, 53, 63, 64, 74, 75, 103-108, preferably in the presence/with of two (e.g., overlapping) hydrolysis probes selected from the group consisting of: SEQ ID NOs: 41-42, SEQ ID NOs: 52-53, 63-64, 74-75.
12. The PCR-method of any one of the preceding items, wherein for the amplification step for human RNase P at least the primer nucleotide sequences set forth in SEQ ID NOs: 7 and 8, SEQ ID NOs: 36-37, 47-48, 58-59, 69-70, 80-81 or 154-155 are used.
13. The PCR-method of any one of the preceding items, wherein for the amplification step for human RNase P at least the primer nucleotide sequences set forth in SEQ ID NOs: 7 and 8, SEQ ID NOs: 36-37, 47-48, 58-59, 69-70, 80-81 or 154-155 are used in the presence of at least one hydrolysis probe selected from the group consisting of: SEQ ID NOs: 38, 49, 60, 71, 82 or 156.
14. The PCR-method of any one of the preceding items, wherein for the amplification step for PB1 of IAV at least the primer nucleotide sequences set forth in SEQ ID NOs: 83-84 or 121-128 are used, preferably SEQ ID NOs: 83-84.
15. The PCR-method of any one of the preceding items, wherein for the amplification step for PB1 of IAV at least the primer nucleotide sequences set forth in SEQ ID NOs: 83-84 or 121-128 are used in the presence of at least one hydrolysis probe selected from the group consisting of: SEQ ID NOs: 85, 129-131, preferably SEQ ID NO: 85.
16. The PCR-method of any one of the preceding items, wherein for the amplification step for PA IBV at least the primer nucleotide sequences set forth in SEQ ID NOs: 86-87 or 132-144 are used, preferably SEQ ID NOs: 86-87.
17. The PCR-method of any one of the preceding items, wherein for the amplification step for PA IBV at least the primer nucleotide sequences set forth in SEQ ID NOs: 86-87 or 132-144 are used in the presence of at least one hydrolysis probe selected from the group consisting of: SEQ ID NOs: 88, 145-153, preferably SEQ ID NO: 88.
18. The PCR-method of any one of the preceding items, wherein for the amplification step for unique spike RNA at least the primer nucleotide sequences set forth in SEQ ID Nos: 17 and 18 are used.
19. A PCR-method comprising conducting an amplification step with at least the primer nucleotide sequences set forth in SEQ ID NOs: 1 and 2, preferably said PCR-method is a multiplex PCR method, further preferably said multiplex PCR method is a multiplex real-time RT-PCR method.
20. The PCR-method of item 9, further comprising conducting an amplification step with at least the primer nucleotide sequences set forth in SEQ ID NOs: 4 or 22 and 5.
21. The PCR-method of item 17 or 18, comprising simultaneously conducting an amplification step with at least the primer nucleotide sequences set forth in SEQ ID NOs: 7 and 8.
22. The PCR-method of any one of the preceding items, wherein for the amplification step for unique spike RNA at least the primer nucleotide sequences set forth in SEQ ID NOs: 17 and 18 are used.
23. The PCR-method of any one of the preceding items, further comprising a probe comprising the nucleotide sequence set forth in SEQ ID NO: 3.
24. The PCR-method of any one of the preceding items, further comprising a probe comprising the nucleotide sequence set forth in SEQ ID NO: 6.
25. The PCR-method of any one of the preceding items, further comprising a probe comprising the nucleotide sequence set forth in SEQ ID NO: 9.
26. The PCR-method of any one of the preceding items, further comprising a probe comprising the nucleotide sequence set forth in SEQ ID NO: 19.
27. The PCR-method of any one of the preceding items, wherein a ribonucleic acid having the sequence set forth in SEQ ID NO: 10 is used as positive control.
28. The PCR-method of any one of the preceding items, wherein a ribonucleic acid having the sequence set forth in SEQ ID NO: 11 or 23 is used as positive control.
29. The PCR-method of any one of the preceding items, wherein a ribonucleic acid having the sequence set forth in SEQ ID NO: 12 is used as positive control.
30. The PCR-method of any one of the preceding items, wherein a ribonucleic acid having the sequence set forth in SEQ ID NO: 20 is used as spike RNA.
31. The PCR-method of any one of the preceding items, which is a real time RT-PCR (e.g., multiplex real time RT-PCR), preferably said real time RT-PCR method is a one-step real time RT-qPCR (RT-quantitative PCR) method (e.g., multiplex one-step real time RT-qPCR method), further preferably said one-step real time RT-qPCR method comprising the following steps:
   (i) incubating a sample at a temperature from about 50° C. to about 55° C. for about 10-30 minutes (e.g.,10, 15, 20, 25 or 30 minutes);
      (ii) incubating said sample at a temperature of about 95° C. for about 3-10 minutes (e.g., 3, 4, 5, 6, 7, 8, 9 or 10 minutes); and/or
      (iii) PCR-amplifying said sample for about 40-45 cycles, wherein each cycle comprising (or consisting of):
         (a) incubating said sample at a temperature of about 95° C. for about 5-15 seconds (e.g., 5, 10 or 15 seconds) and
         (b) incubating said sample at a temperature from about 58° C. to about 60° C. for about 20-30 seconds (e.g., 20, 25 or 30 seconds).
32. The PCR-method of any one of the preceding items, which is a multiplex PCR, preferably a multiplex real-time RT-PCR.
33. A kit comprising at least the primer nucleotide sequences set forth in SEQ ID NOs: 1 and 2 or SEQ ID NOs: 27-28, SEQ ID NOs: 33-34, SEQ ID NOs: 43-44, SEQ ID NOs: 54-55, SEQ ID NOs: 65-66, SEQ ID NOs: 76-77, SEQ ID NOs: 109-110, SEQ ID NOs: 109 and 111 or SEQ ID NOs: 109 and 112, optionally at least one probe comprising the nucleotide sequence set forth in SEQ ID NO: 3 or SEQ ID NOs: 29, 35, 45, 46, 56, 57, 67, 68, 78, 79 and 113-120, preferably comprising two (e.g., overlapping) probes selected from the group consisting of: SEQ ID NOs: 45-46, SEQ ID NOs: 56-57, 67-68, 78-79 and optionally means for carrying out a PCR amplification step, preferably said kit is a multiplex PCR kit, preferably said multiplex PCR kit is a multiplex real-time RT-PCR kit.
34. The kit of item 31, further comprising at least the primer nucleotide sequences set forth in SEQ ID NOs: 4 or 22 and 5 or SEQ ID NOs: 24-25, SEQ ID NOs: 30-31, SEQ ID NOs: 39-40, 50-51, 61-62, 72-73, any one of 89-101 with 102, and optionally at least one probe comprising the nucleotide sequence set forth in SEQ ID NO: 6 or SEQ ID NOs: 26, 32, 41, 42, 52, 53, 63, 64, 74, 75, 103-108, preferably comprising two 8e.g., overlapping) probes selected from the group consisting of: SEQ ID NOs: 41-42, SEQ ID NOs: 52-53, 63-64, 74-75.
35. The kit of any one of the preceding items, further comprising at least the primer nucleotide sequences set forth in SEQ ID NOs: 7 and 8, or SEQ ID NOs: 36-37, 47-48, 58-59, 69-70, 80-81 or 154-155, and optionally at least one probe comprising the nucleotide sequence set forth in SEQ ID NO: 9 or SEQ ID NOs: 38, 49, 60, 71, 82 or 156.
36. The kit of any one of the preceding items, further comprising at least the primer nucleotide sequences set forth in SEQ ID NOs: 83-84 or 121-128, preferably in SEQ ID NOs: 83-84, optionally at least one probe selected from the group consisting of: SEQ ID NOs: 85, 129-131, further preferably SEQ ID NO: 85.
36. The kit of any one of the preceding items, further comprising at least the primer nucleotide sequences set forth in SEQ ID NOs: 86-87 or 132-144 are used, preferably SEQ ID NOs: 86-87, optionally at least one probe selected from the group consisting of: SEQ ID NOs: 88, 145-153, further preferably SEQ ID NO: 88.
37. The kit of any one of the preceding items, further comprising at least the primer nucleotide sequences set forth in SEQ ID NOs: 17 and 18, and optionally a probe comprising the nucleotide sequence set forth in SEQ ID NO: 19.
38. The kit of any one of the preceding items, further comprising a ribonucleic acid having the sequence set forth in SEQ ID NO: 10, SEQ ID NO: 11 or 23, SEQ ID NO: 12 and SEQ ID NO: 20.
39. Use of the primer nucleotide sequences, wherein said primer sequences:
   (a) set forth in SEQ ID NOs: 1-2 or SEQ ID NOs: 27-28, SEQ ID NOs: 33-34, SEQ ID NOs: 43-44, SEQ ID NOs: 54-55, SEQ ID NOs: 65-66, SEQ ID NOs: 76-77, SEQ ID NOs: 109-110, SEQ ID NOs: 109 and 111 SEQ ID NOs: 109 and 112, preferably with at least one (e.g., two) probe selected from the group consisting of: SEQ ID NOs: 29, 35, 45, 46, 56, 57, 67, 68, 78, 79 and 113-120, further preferably with two partially overlapping probes (e.g., non-identical probes) selected from the group consisting of: SEQ ID NOs: 45-46, SEQ ID NOs: 56-57, 67-68, 78-79; and/or
   (b) set forth in SEQ ID NOs: 4 or 22 and 5, or SEQ ID NOs: 24-25, SEQ ID NOs: 30-31, SEQ ID NOs: 39-40, 50-51, 61-62, 72-73, any one of 89-101 with 102, preferably with at least one (e.g., two) probe selected from the group consisting of: SEQ ID NOs: 26, 32, 41, 42, 52, 53, 63, 64, 74, 75, 103-108, further preferably with two probes selected from: SEQ ID NOs: 41-42, SEQ ID NOs: 52-53, 63-64, 74-75; and/or
   (c) set forth in SEQ ID NOs: 83-84 or 121-128, preferably SEQ ID NOs: 83-84, further preferably with at least one probe selected from the group consisting of: SEQ ID NOs: 85, 129-131, most preferably SEQ ID NO: 85;
   (d) set forth in SEQ ID NOs: 86-87 or 132-144, preferably SEQ ID NOs: 86-87, further preferably with at least one probe selected from the group consisting of: SEQ ID NOs: SEQ ID NOs: 88, 145-153, preferably SEQ ID NO: 88;
   wherein said sequences of (a), (b), (c) and (d) are used alone or in combination with one another for one or more of the following:
   (i) in vitro detection (e.g., simultaneous, e.g., multiplexed detection) of SARS-CoV-2 and/or IAV and/or IBV in a sample,
   (ii) in vitro detection (e.g., simultaneous, e.g., multiplexed detection) of an infection of a subject with SARS-CoV-2 and/or IAV and/or IBV,
   (iii) in vitro detection (e.g., simultaneous, e.g., multiplexed detection) of a contamination of a blood sample with SARS-CoV-2 and/or IAV and/or IBV, or
   (iv) monitoring (e.g., simultaneous, e.g., multiplexed monitoring) the therapy of SARS-CoV-2 and/or IAV and/or IBV in vitro,
      wherein said use is the use for multiplex PCR detection, preferably a multiplex real-time RT-PCR detection;
   (v) for/in the PCR-method according of any one of the preceding claims, preferably said method is an in vitro or ex vivo method.
40. The use of any one of the preceding item, wherein further a probe comprising the nucleotide sequence set forth in SEQ ID NO: 3 is used for the in vitro detection.
41. The PCR-method, kit or use according to any one of the preceding items, wherein said PCR-method is a multiplex real time RT-PCR method, preferably a one-step multiplex real time RT-qPCR (RT-quantitative PCR) method, further preferably said one-step multiplex real time RT-qPCR method comprising the following steps:
   (i) incubating a sample at a temperature from about 50° C. to about 55° C. for about 10-30 minutes (e.g.,10, 15, 20, 25 or 30 minutes);
   (ii) incubating said sample at a temperature of about 95° C. for about 3-10 minutes (e.g., 3, 4, 5, 6, 7, 8, 9 or 10 minutes); and/or
   (iii) PCR-amplifying said sample for about 40-45 cycles, wherein each cycle comprising (or consisting of):
      (a) incubating said sample at a temperature of about 95° C. for about 5-15 seconds (e.g., 5, 10 or 15 seconds) and
      (b) incubating said sample at a temperature from about 58° C. to about 60° C. for about 20-30 seconds (e.g., 20, 25 or 30 seconds).
42. The PCR-method, kit or use according to any one of the preceding items, wherein said primer/s and/or probe/s nucleotide sequence/s comprising one or more (e.g., 2, 3 or 4) Locked Nucleic Acids (LNA)-modified nucleotides (e.g., LNA is a synthetic nucleic acid analogue containing a bridged, bicyclic sugar moiety, e.g., a methylene linkage between the 2′ oxygen and the 4′ carbon of the ribose ring), preferably said one or more (e.g., 2, 3 or 4) LNA-modified nucleotides are capable of stabilizing either the 3′- and/or 5′-end, stabilizing a mismatched base for allelic (e.g., SNP) discrimination, and/or increasing the melting temperatures of the said primer/s and/or probe, further preferably said one or more (e.g., 2, 3 or 4) LNA-modified nucleotides are LNA-modified thymine residues (e.g., LNA-T) and/or LNA-modified adenosine residues (e.g., LNA-A).
43. The PCR-method, kit or use according to any one of the preceding items, comprising conducting a simultaneous (e.g., multiplexed) amplification step with at least one probe, preferably said probe is derivatized with a fluorophore (e.g., Fluorescein (FAM), Yakima Yellow (YY), Cy5, HEX) at 5′-end and a quencher at a 3′-end capable of absorbing energy without emitting fluorescence (e.g., Black Hole Quencher 1, 2, 3 (BHQ1, BHQ2, BHQ3).
44. The PCR-method, kit or use according to any one of the preceding items, comprising conducting a simultaneous (e.g., multiplexed) amplification step with two probes, preferably said probes are derivatized with a fluorophore (e.g., Fluorescein (FAM), Yakima Yellow (YY), Cy5, HEX) at 5′-end and a quencher at a 3′-end capable of absorbing energy without emitting fluorescence (e.g., Black Hole Quencher 1, 2, 3 (BHQ1, BHQ2, BHQ3).
45. The PCR-method, kit or use according to any one of the preceding items, wherein said PCR-method, kit or use comprising one or more primer or/and probe nucleotide sequences selected from the group consisting of: SEQ ID NOs: 1-156, preferably said probe nucleotide sequence is derivatized with a fluorophore (e.g., Fluorescein (FAM), Yakima Yellow (YY), Cy5, HEX) at 5′-end and a quencher at a 3′-end capable of absorbing energy without emitting fluorescence (e.g., Black Hole Quencher 1, 2, 3 (BHQ1, BHQ2, BHQ3).
46. The PCR-method, kit or use according to any one of the preceding items, comprising addition of nucleic acid decoys (e.g., to PCR-reaction mixture, e.g., a positive control PCR-reaction mixture, e.g., lyophilized positive control PCR-reaction mixture), preferably in the form of baker's yeast tRNA and/or salmon sperm DNA.
47. The PCR-method, kit or use according to any one of the preceding items, wherein said PCR-method, kit or use is a diagnostic PCR-method, kit or use.
48. The PCR-method, kit or use according to any one of the preceding items, wherein said PCR-method, kit or use is an in vitro or ex vivo PCR-method, kit or use.
49. The PCR-method, kit or use according to any one of the preceding items, wherein said PCR-method, kit or use is for diagnosing a viral disease, preferably said viral disease is selected from the group consisting of: SARS-CoV-2, Influenza virus A (IAV) and Influenza virus B (IBV), further preferably for simultaneous (e.g., multiplexed) diagnosing SARS-CoV-2, Influenza virus A (IAV) and/or Influenza virus B (IBV).
50. The PCR-method, kit or use according to any one of the preceding items, wherein said primer and/or probe nucleotide sequence/s comprising one or more (e.g., 2, 3 or 4) Locked Nucleic Acids (LNA)-modified nucleotides (e.g., LNA is a synthetic nucleic acid analogue containing a bridged, bicyclic sugar moiety, e.g., a methylene linkage between the 2′ oxygen and the 4′ carbon of the ribose ring), preferably said one or more (e.g., 2, 3 or 4) LNA-modified nucleotides are LNA-modified thymine residues (e.g., LNA-T) and/or LNA-modified adenosine residues (e.g., LNA-A).
51. The PCR-method, kit or use according to any one of the preceding items, further comprising conducting a simultaneous amplification step with at least primer nucleotide sequences targeting the spike (S) gene of SARS-CoV-2, preferably with primers having SEQ ID NOs: 157-158, preferably with a probe/s having SEQ ID NO: 161 or 162.
52. The PCR-method, kit or use according to any one of the preceding items, further comprising conducting a simultaneous amplification step with at least primer nucleotide sequences targeting two mutations (deletions) that are present in the B.1.1.7 variant/mutant of SARS-CoV-2, preferably with primers having SEQ ID NOs: 159-160, preferably with a probe/s having SEQ ID NO: 161 or 162.
53. A primer and/or probe nucleotide sequence comprising or consisting of a nucleotide sequence selected from the group consisting of: SEQ ID NOs: 1-162, preferably said probe nucleotide sequence is derivatized with a fluorophore (e.g., Fluorescein (FAM), Yakima Yellow (YY), Cy5, HEX) at 5′-end and a quencher at a 3′-end capable of absorbing energy without emitting fluorescence (e.g., Black Hole Quencher 1, 2, 3 (BHQ1, BHQ2, BHQ3).
54. The primer and/or probe nucleotide sequence according to any one of the preceding items, wherein said primer/s and/or probe/s nucleotide sequence/s comprising one or more (e.g., 2, 3 or 4) Locked Nucleic Acids (LNA)-modified nucleotides (e.g., LNA is a synthetic nucleic acid analogue containing a bridged, bicyclic sugar moiety, e.g., a methylene linkage between the 2′ oxygen and the 4′ carbon of the ribose ring), preferably said one or more (e.g., 2, 3 or 4) LNA-modified nucleotides are capable of stabilizing either the 3′- and/or 5′-end, stabilizing a mismatched base for allelic (e.g., SNP) discrimination, and/or increasing the melting temperatures of the said primer/s and/or probe, further preferably said one or more (e.g., 2, 3 or 4) LNA-modified nucleotides are LNA-modified thymine residues (e.g., LNA-T) and/or LNA-modified adenosine residues (e.g., LNA-A).
55. The primer and/or probe nucleotide sequence according to any one of the preceding items for use in a method of diagnosing a viral disease.
56. The primer and/or probe nucleotide sequence according to any one of the preceding items for use in a method of diagnosing a viral disease, wherein said viral disease is selected from the group consisting of: SARS-CoV-2, Influenza virus A (IAV) and Influenza virus B (IBV), preferably in a method of simultaneous (e.g., multiplexed) diagnosing SARS-CoV-2, Influenza virus A (IAV) and/or Influenza virus B (IBV).
57. The primer and/or probe nucleotide sequence according to any one of the preceding items for use in a method according to any one of the preceding items, preferably wherein said method is a method of diagnosing a viral disease, further preferably said viral disease is selected from the group consisting of: SARS-CoV-2, Influenza virus A (IAV) and Influenza virus B (IBV), most preferably said method is a method of simultaneous (e.g., multiplexed) diagnosing SARS-CoV-2, Influenza virus A (IAV) and/or Influenza virus B (IBV).

Unless otherwise stated, the following terms used in this document, including the description and claims, have the definitions given below.

Those skilled in the art will recognize, or be able to ascertain, using not more than routine experimentation, many equivalents to the specific embodiments of the invention described herein. Such equivalents are intended to be encompassed by the present invention.

It is to be noted that as used herein, the singular forms “a”, “an”, and “the”, include plural references unless the context clearly indicates otherwise. Thus, for example, reference to “a reagent” includes one or more of such different reagents and reference to “the method” includes reference to equivalent steps and methods known to those of ordinary skill in the art that could be modified or substituted for the methods described herein.

Unless otherwise indicated, the term “at least” preceding a series of elements is to be understood to refer to every element in the series. Those skilled in the art will recognize or be able to ascertain using no more than routine experimentation, many equivalents to the specific embodiments of the invention described herein. Such equivalents are intended to be encompassed by the present invention.

The term “and/or” wherever used herein includes the meaning of “and”, “or” and “all or any other combination of the elements connected by said term”.

The term “about” or “approximately” as used herein means within 20%, preferably within 10%, and more preferably within 5% of a given value or range. It includes, however, also the concrete number, e.g., about 20 includes 20.

Throughout this specification and the claims which follow, unless the context requires otherwise, the word “comprise”, and variations such as “comprises” and “comprising”, will be understood to imply the inclusion of a stated integer or step or group of integers or steps but not the exclusion of any other integer or step or group of integer or step. When used herein the term “comprising” can be substituted with the term “containing” or “including” or sometimes when used herein with the term “having”.

When used herein “consisting of” excludes any element, step, or ingredient not specified in the claim element. When used herein, “consisting essentially of” does not exclude materials or steps that do not materially affect the basic and novel characteristics of the claim.

In each instance herein any of the terms “comprising”, “consisting essentially of” and “consisting of” may be replaced with either of the other two terms.

It should be understood that this invention is not limited to the particular methodology, protocols, material, reagents, and substances, etc., described herein and as such can vary. The terminology used herein is for the purpose of describing particular embodiments only, and is not intended to limit the scope of the present invention, which is defined solely by the claims.

All publications cited throughout the text of this specification (including all patents, patent applications, scientific publications, manufacturer's specifications, instructions, etc.) are hereby incorporated by reference in their entirety. Nothing herein is to be construed as an admission that the invention is not entitled to antedate such disclosure by virtue of prior invention. To the extent the material incorporated by reference contradicts or is inconsistent with this specification, the specification will supersede any such material.

### EXAMPLES

The following examples illustrate the invention. These examples should not be construed as to limit the scope of this invention. The examples are included for purposes of illustration and the present invention is limited only by the claims.

### Example 1

### Real-Time RT-PCR for Detecting SARS-CoV-2 Genes

Synthetic SARS-CoV-2 RNA was obtained from Exact Diagnostics (http://www.exactdiagnostics.com/sars-cov-2-standard.html). Real-time RT-PCR was carried out using the Brilliant III Ultra-Fast qRT-PCR Master Mix (Agilent, Catalog #600884). Reaction mixtures were prepared according to the manufacturer's specifications: 1× qRT-PCR master mix, 1 mM DTT, 30 nM ROX reference dye, 1 μl of RT/RNase block (concentration not specified by the supplier), 2 μl of mix of oligos (final concentration in qRT-PCR master mix for E gene: 400 nM forward primer, 400 nM reverse primer, 200 nM probe; final concentration in qRT-PCR master mix for RdRP gene: 600 nM forward primer, 800 nM reverse primer, 100 nM probe), 5 μl of synthetic RNA (E, N, S, ORF1ab, and RdRP transcripts of SARS-CoV-2, 200,000 copies/ml each), nuclease-free PCR-grade H₂O to the final volume of 20 μl. Reactions were performed in triplicates and ROX was used as a passive reference dye. Real-time RT-PCR was subsequently performed on Agilent Stratagene Mx3005P real-time PCR instrument (Agilent Technologies) under the following conditions: 50° C. for 10 min as cDNA synthesis (reverse transcription), 95° C. for 3 min as initial denaturation, followed by 45 cycles of 95° C. for 10 sec and 58° C. for 20 sec, with a fluorescence detection performed during each annealing/extension step. Data was acquired and analyzed using the Mx3005P software (Agilent Technologies).

Table 1 shows the tests and the corresponding results when conducting the PCR-method according to the present invention by using the primers and probes as indicated beyond Table 1.

| | | | Threshold | | |
|---|---|---|---|---|---|
| Well | Well Name | Replicate | (dRn) | Ct (dRn) | average |
| A3 | E | 1 | 0.01 | 30.23 | 30.21 |
| B3 | E | 1 | 0.01 | 30.29 | |
| C3 | E | 1 | 0.01 | 30.11 | |
| E3 | R1 MDX | 2 | 0.01 | 33.01 | 32.89 |
| F3 | R1 MDX | 2 | 0.01 | 32.89 | |
| G3 | R1 MDX | 2 | 0.01 | 32.77 | |
| A6 | R2 MDX | 3 | 0.01 | 30.85 | 31.17 |
| B6 | R2 MDX | 3 | 0.01 | 31.42 | |
| C6 | R2 MDX | 3 | 0.01 | 31.24 | |
| E6 | R2 new MDX | 4 | 0.01 | 31.13 | 31.18 |
| F6 | R2 new MDX | 4 | 0.01 | 31.22 | |
| G6 | R2 new MDX | 4 | 0.01 | 31.19 | |
| A9 | P1 eurofins | 5 | 0.01 | 44.76 | No Ct |
| B9 | P1 eurofins | 5 | 0.01 | no Ct | |
| C9 | P1 eurofins | 5 | 0.01 | no Ct | |
| E9 | P2 eurofins | 6 | 0.01 | 34.18 | 34.26 |
| F9 | P2 eurofins | 6 | 0.01 | 34.27 | |
| G9 | P2 eurofins | 6 | 0.01 | 34.33 | |
| A11 | R2 new MDX ntc | 7 | 0.01 | no Ct | no Ct |
| B11 | R2 new MDX ntc | 7 | 0.01 | no Ct | |
| C11 | R2 new MDX ntc | 7 | 0.01 | no Ct | |

E=SARS-CoV-2 E gene primer nucleotide sequences shown in SEQ ID NO: 4 and 5, probe shown In SEQ ID NO: 6 (curve within FIG. 1 is marked by filled circles)
R1 MDX=SARS-CoV-2 RdRP primer nucleotide sequences shown in SEQ ID NO: 1 and SEQ ID NO: 13, probe shown in SEQ ID NO: 3 (curve within FIG. 1 is marked by filled squares)
R2 MDX=SARS-CoV-2 RdRP primer nucleotide sequences shown in SEQ ID NO: 1 and SEQ ID NO: 2, probe shown in SEQ ID NO: 3 (curve within FIG. 1 is marked by filled triangles)
R2 new MDX=SARS-CoV-2 RdRP primer nucleotide sequences shown in SEQ ID NO: 1 and SEQ ID NO: 2, probe shown in SEQ ID NO: 3 (same as “R2 MDX”, except for the use of a new batch of synthesized primer nucleotide sequences) (curve within FIG. 1 is marked by filled diamonds)
P1 eurofins=SARS-CoV-2 RdRP primer nucleotide sequences shown in SEQ ID NO: 14 and SEQ ID NO: 15, probe shown in SEQ ID NO: 16 (curve within FIG. 1 is marked by filled stars)
P2 eurofins=SARS-CoV-2 RdRP primer nucleotide sequences shown in SEQ ID NO: 14 and SEQ ID NO: 15, probe shown in SEQ ID NO: 3 (curve within FIG. 1 is marked by open diamonds)
R2 new MDX=SARS-CoV-2 RdRP primer nucleotide sequences shown in SEQ ID NO: 1 and SEQ ID NO: 2 (curve within FIG. 1 is marked by open squares)

It is apparent from FIG. 1 that the detection of SARS-CoV-2 E gene by RT-PCR using primer nucleotide sequences (SEQ ID NOs: 4 and 5) and a probe having SEQ ID NO: 6—well name “E” in Table 1—works properly (Ct (dRN)=30.21) (curve in FIG. 1 is marked by filled circles).

With respect to the detection of SARS-CoV-2 RdRP gene, the present inventors observed major specificity issues when using published primer nucleotide sequences (SEQ ID NOs: 14 and 15) and a probe (SEQ ID NO: 16)—well name “P1 eurofins” in Table 1. Indeed, they did not observe a Ct (dRn). The corresponding curve in FIG. 1 is marked by filled stars.

The present inventors tackled this specificity issue in different ways and could successfully resolve it.

First, they converted all degenerate nucleotides of the probe having SEQ ID NO: 16 into non-degenerate, i.e., specific nucleotides, thereby obtaining the probe having SEQ ID NO: 3. Using primer nucleotide sequences (SEQ ID NOs: 14 and 15) and the probe having SEQ ID NO: 3—well name “P2 eurofins” in Table 1—the specificity (Ct (dRN)=34.26) was improved in contrast to “P1 eurofins” (no Ct (dRN) at all). The corresponding curve in FIG. 1 is marked by open diamonds.

Second, since the specificity of 34.26 was still not satisfying, the present inventors converted the degenerate nucleotides of the primer nucleotide sequence of SEQ ID NOs: 14 and 15 into non-degenerate, i.e., specific nucleotides, thereby obtaining the primer nucleotide sequence having SEQ ID NOs: 1 and 13, respectively. Using these primer nucleotide sequences (SEQ ID NOs: 1 and 13) together with the probe having SEQ ID NO: 3—well name “R1 MDX” in Table 1—the specificity (Ct (dRN)=32.89) was further improved in contrast to “P2 eurofins” (Ct (dRN)=34.26). The corresponding curve in FIG. 1 is marked by filled squares.

Third, in order to try to further improve the specificity of “R1 MDX” (see Table 1), the present inventors observed that the primer nucleotide sequences having SEQ ID NOs: 1 and 2, which when used together with the probe having SEQ ID NO: 3—well name “R2 MDX” and “R2 new MDX” in Table 1—confer for an even improved specificity (Ct (dRN)=31.17 and 31.18) in contrast to “R1 MDX” (Ct (dRN)=32.89) (see Table 1). The corresponding curve in FIG. 1 is marked by filled triangles and filled diamonds, respectively.

As a negative control, the primer nucleotide sequences having SEQ ID NOs: 1 and 2, but no probe was used—well name “R2 new MDX ntc”. The corresponding curve in FIG. 1 is marked by open squares.

Thus, the present inventors found that primer nucleotide sequences having SEQ ID NOs: 1 and 2, optionally together with a probe having SEQ ID NO: 3 provides for an improved specificity much improved specificity as regards the detection of SARS-CoV-2 RdRP gene vis-à-vis the commonly used primer nucleotide sequences having SEQ ID NOs: 14 and 15, optionally together with a probe having SEQ ID NO: 3 or 16.

Comparing a Ct of 30.21 for SARS-CoV-2 E gene with a Ct of 31.17 and 31.18 for SARS-CoV-2 RdRP, it is apparent that there is a difference of less than one Ct. This is a significant improvement of a method for the detection of SARS-CoV-2 as described in the present application.

Of note, it is apparent that the primer nucleotide sequences of SEQ ID NOs: 1 and 2 cannot only be used for RT-PCR, e.g., real-time RT-PCR, but also for a well-known standard PCR, e.g., RT-LAMP.

### Example 2

### Development of a Room Temperature-Stable, Multiplexed RT-qPCR Test Using Dual Probe Technology to Differentiate SARS-CoV-2 from Influenza A and B

Materials and Methods

vDetect v1

RT-qPCR reactions were optimized on a CFX96 (Bio-Rad) and Mx3005P (Agilent Technologies) real time PCR detection systems using the 1Step RT qPCR Probe ROX L Kit (Cat. No. QOP0201, highQu, Germany). For E gene and RdRP gene detection, the reaction mixture prepared according to the manufacturer's recommendations comprised 10 μl of 2× HighQu Master Mix, 2 μl of RT3 Mix, 2 μl of primers/probe mix, 1 μl of PCR water, and 5 μl of sample in a 20 μl total volume. One-step RT-qPCR assays were conducted with the following cycling conditions: 50° C. for 10 min for reverse transcription, 95° C. for 3 min, and 45 cycles of 95° C. for 5 s and 60° C. for 20 s. The primer pairs and probes sequences are show in Table 2 and Table 3.

vDetect v2

RT-qPCR reactions were optimized on a CFX96 (Bio-Rad), Mx3005P (Agilent Technologies) and AriaMx (Agilent Technologies) real time PCR detection systems using Brilliant III Ultra-Fast QRT-PCR Master Mix (Cat. No. 600884; Agilent Technologies). For E gene, RdRP gene and RNase P gene detection, the reaction mixture prepared according to the manufacturer's recommendations comprised 10 μl of 2× Brilliant III Ultra-Fast QRT-PCR Master Mix, 0.3 μl of 2 μM ROX, 0.2 μl of 100 mM DTT, 1 μl of RT/RNase Block, 2 μl of primers/probe mix, 1.5 μl of PCR water, and 5 μl of sample in a 20 μl total volume. One-step RT-qPCR assays were conducted with the following cycling conditions: 50° C. for 30 min for reverse transcription, 95° C. for 3 min, and 45 cycles of 95° C. for 5 s and 60° C. for 20 s. The primer pairs and probes sequences are show in Table 2 and Table 3.

rTEST Singleplex, Multiplex and Allplex

RT-qPCR reactions were optimized on a Mx3005P (Agilent, CA, USA) and AriaMx (Agilent, CA, USA) real time PCR detection systems using SOLIScript® 1-step CoV Kit (Cat. No. 08-65-00250; SOLIS BioDyne, Estonia). For all the detected genes, the reaction mixture prepared according to the manufacturer's recommendations comprised 4 μl of 5× One-step Probe CoV Mix (ROX), 0.5 μl of 40× One-step SOLIScript® CoV Mix, 2 μl of primers/probe mix, 8.5 μl of PCR water, and 5 μl of sample in a 20 μl total volume. One-step RT-qPCR assays were conducted with the following cycling conditions: 55° C. for 10 min for reverse transcription, 95° C. for 10 min, and 45 cycles of 95° C. for 15 s and 60° C. for 30 s. The primer pairs and probes sequences are show in Table 2 and Table 3.

TABLE 2
Primers and probes used for SARS-CoV-2, IAV and IBV detection.

| | Primer/probe | Sequence (5′ - 3′) in combination with the | Concentration |
|---|---|---|---|
| Kit | name | preferred label/s | (nM) |
| vDetect | SEQ ID NO: 24 | GTACTCATTCGTTTCGGAAGAGACAG | 500 |
| v1. | E_Sarbeco_F3 | | |
| | SEQ ID NO: 25 | ATATTGCAGCAGTACGCACACA | 500 |
| | E_Sarbeco_R2 | | |
| | SEQ ID NO: 26 | FAM-ACACTAGCCATCCTTACTGCGCTTCG- | 200 |
| | E_Sarbeco_P1 | BHQ1 | |
| | SEQ ID NO: 27 | GTGAAATGGTCATGTGTGGCGG | 600 |
| | RdRP_SARSr- | | |
| | F2 | | |
| | SEQ ID NO: 28 | CGTGACAGCTTGACAAATGTTAAAAAC | 800 |
| | RdRP_SARSr- | | |
| | R2 | | |
| | SEQ ID NO: 29 | FAM-CAGGTGGAACCTCATCAGGAGATGC- | 200 |
| | RdRP_SARSr- | BHQ1 | |
| | P2 | | |
| vDetect | SEQ ID NO: 30 | AtGtACTCATTCGTTTCGGAAGA | 500 |
| v2. | E_Sarbeco_F7 | | |
| | with LNA-Ts (t) | | |
| | SEQ ID NO: 31 | ATATTGCAGCAGTACGCACACA | 500 |
| | E_Sarbeco_R2 | | |
| | SEQ ID NO: 32 | FAM-ACACTAGCCATCCTTACTGCGCTTCG- | 200 |
| | E_Sarbeco_P1 | BHQ1 | |
| | SEQ ID NO: 33 | GTGAAATGGTCATGTGTGGCGG | 600 |
| | RdRP_SARSr- | | |
| | F2 | | |
| | SEQ ID NO: 34 | CGTGACAGCTTGACAAATGTTAAAAAC | 800 |
| | RdRP_SARSr- | | |
| | R2 | | |
| | SEQ ID NO: 35 | FAM-TCAGGAGATGCCACAACTGCTTATGC- | 200 |
| | RdRP_SARSr- | BHQ1 | |
| | P8 | | |
| | SEQ ID NO: 36 | AGATTTGGACCTGCGAGCG | 500 |
| | RNaseP | | |
| | Forward | | |
| | SEQ ID NO: 37 | GAGCGGCTGTCTCCACAAGT | 500 |
| | RNaseP | | |
| | Reverse | | |
| | SEQ ID NO: 38 | FAM-TTCTGACCTGAAGGCTCTGCGCG- | 160 |
| | RNase P Probe | BHQ1 | |
| | 1 | | |
| rTEST | SEQ ID NO: 39 | AtGtACTCATTCGTTTCGGAAGA | 500 |
| | E_Sarbeco_F7 | | |
| | with LNA-Ts (t) | | |
| | SEQ ID NO: 40 | ATATTGCAGCAGTACGCACACA | 500 |
| | E_Sarbeco_R2 | | |
| | SEQ ID NO: 41 | FAM-ACACTAGCCATCCTTACTGCGCTTCG- | 200 |
| | E_Sarbeco_P1 | BHQ1 | |
| | SEQ ID NO: 42 | FAM- | 200 |
| | E_Sarbeco_P2 | TAGCGTACTTCTTTTTCTTGCTTTCGTGGT- | |
| | | BHQ1 | |
| | SEQ ID NO: 43 | GTGAAATGGTCATGTGTGGCGG | 600 |
| | RdRP_SARSr- | | |
| | F2 | | |
| | SEQ ID NO: 44 | CGTGACAGCTTGACAAATGTTAAAAAC | 800 |
| | RdRP_SARSr- | | |
| | R2 | | |
| | SEQ ID NO: 45 | FAM-CAGGTGGAACCTCATCAGGAGATGC- | 200 |
| | RdRP_SARSr- | BHQ1 | |
| | P2 | | |
| | SEQ ID NO: 46 | FAM-TCAGGAGATGCCACAACTGCTTATGC- | 200 |
| | RdRP_SARSr- | BHQ1 | |
| | P8 | | |
| | SEQ ID NO: 47 | AGATTTGGACCTGCGAGCG | 500 |
| | RNaseP | | |
| | Forward | | |
| | SEQ ID NO: 48 | GAGCGGCTGTCTCCACAAGT | 500 |
| | RNaseP | | |
| | Reverse | | |
| | SEQ ID NO: 49 | FAM-TTCTGACCTGAAGGCTCTGCGCG- | 160 |
| | RNase P Probe | BHQ1 | |
| | 1 | | |
| rTEST | SEQ ID NO: 50 | AtGtACTCATTCGTTTCGGAAGA | 500 |
| Multiple | E_Sarbeco_F7 | | |
| X | with LNA-Ts (t) | | |
| | SEQ ID NO: 51 | ATATTGCAGCAGTACGCACACA | 500 |
| | E_Sarbeco_R2 | | |
| | SEQ ID NO: 52 | FAM-ACACTAGCCATCCTTACTGCGCTTCG- | 187.5 |
| | E_Sarbeco_P1 | BHQ1 | |
| | SEQ ID NO: 53 | FAM- | 187.5 |
| | E_Sarbeco_P2 | TAGCGTACTTCTTTTTCTTGCTTTCGTGGT- | |
| | | BHQ1 | |
| | SEQ ID NO: 54 | GTGAAATGGTCATGTGTGGCGG | 600 |
| | RdRP_SARSr- | | |
| | F2 | | |
| | SEQ ID NO: 55 | CGTGACAGCTTGACAAATGTTAAAAAC | 800 |
| | RdRP_SARSr- | | |
| | R2 | | |
| | SEQ ID NO: 56 | FAM-CAGGTGGAACCTCATCAGGAGATGC- | 187.5 |
| | RdRP_SARSr- | BHQ1 | |
| | P2 | | |
| | SEQ ID NO: 57 | FAM-TCAGGAGATGCCACAACTGCTTATGC- | 187.5 |
| | RdRP_SARSr- | BHQ1 | |
| | P8 | | |
| | SEQ ID NO: 58 | AGATTTGGACCTGCGAGCG | 312.5 |
| | RNaseP | | |
| | Forward | | |
| | SEQ ID NO: 59 | GAGCGGCTGTCTCCACAAGT | 312.5 |
| | RNaseP | | |
| | Reverse | | |
| | SEQ ID NO: 60 | YY-TTCTGACCTGAAGGCTCTGCGCG-BHQ2 | 87.5 |
| | RNase P Probe | | |
| | 1.1 | | |
| rTEST | SEQ ID NO: 61 | AtGtACTCATTCGTTTCGGAAGA | 500 |
| Allplex | E_Sarbeco_F7 | | |
| | with LNA-Ts (t) | | |
| | SEQ ID NO: 62 | ATATTGCAGCAGTACGCACACA | 500 |
| | E_Sarbeco_R2 | | |
| | SEQ ID NO: 63 | YY-ACACTAGCCATCCTTACTGCGCTTCG- | 200 |
| | E_Sarbeco_P1. | BHQ2 | |
| | 2 | | |
| | SEQ ID NO: 64 | YY- | 200 |
| | E_Sarbeco_P2. | TAGCGTACTTCTTTTTCTTGCTTTCGTGGT- | |
| | 2 | BHQ2 | |
| | SEQ ID NO: 65 | GTGAAATGGTCATGTGTGGCGG | 600 |
| | RdRP_SARSr- | | |
| | F2 | | |
| | SEQ ID NO: 66 | CGTGACAGCTTGACAAATGTTAAAAAC | 800 |
| | RdRP_SARSr- | | |
| | R2 | | |
| | SEQ ID NO: 67 | FAM-CAGGTGGAACCTCATCAGGAGATGC- | 200 |
| | RdRP_SARSr- | BHQ1 | |
| | P2 | | |
| | SEQ ID NO: 68 | FAM-TCAGGAGATGCCACAACTGCTTATGC- | 200 |
| | RdRP_SARSr- | BHQ1 | |
| | P8 | | |
| | SEQ ID NO: 69 | AGATTTGGACCTGCGAGCG | 250 |
| | RNase P | | |
| | Forward | | |
| | SEQ ID NO: 70 | GAGCGGCTGTCTCCACAAGT | 250 |
| | RNase P | | |
| | Reverse | | |
| | SEQ ID NO: 71 | Cy5-TTCTGACCTGAAGGCTCTGCGCG- | 80 |
| | RNase P Probe | BHQ3 | |
| | 1.2 | | |
| rTEST | SEQ ID NO: 72 | AtGtACTCATTCGTTTCGGAAGA | 500 |
| CoV/Flu | E_Sarbeco_F7 | | |
| | with LNA-Ts (t) | | |
| | SEQ ID NO: 73 | ATATTGCAGCAGTACGCACACA | 500 |
| | E_Sarbeco_R2 | | |
| | SEQ ID NO: 74 | FAM-ACACTAGCCATCCTTACTGCGCTTCG- | 200 |
| | E_Sarbeco_P1 | BHQ1 | |
| | SEQ ID NO: 75 | FAM- | 200 |
| | E_Sarbeco_P2 | TAGCGTACTTCTTTTTCTTGCTTTCGTGGT- | |
| | | BHQ1 | |
| | SEQ ID NO: 76 | GTGAAATGGTCATGTGTGGCGG | 600 |
| | RdRP_SARSr- | | |
| | F2 | | |
| | SEQ ID NO: 77 | CGTGACAGCTTGACAAATGTTAAAAAC | 800 |
| | RdRP_SARSr- | | |
| | R2 | | |
| | SEQ ID NO: 78 | FAM-CAGGTGGAACCTCATCAGGAGATGC- | 200 |
| | RdRP_SARSr- | BHQ1 | |
| | P2 | | |
| | SEQ ID NO: 79 | FAM-TCAGGAGATGCCACAACTGCTTATGC- | 200 |
| | RdRP_SARSr- | BHQ1 | |
| | P8 | | |
| | SEQ ID NO: 80 | AGATTTGGACCTGCGAGCG | 250 |
| | RNaseP | | |
| | Forward | | |
| | SEQ ID NO: 81 | GAGCGGCTGTCTCCACAAGT | 250 |
| | RNaseP | | |
| | Reverse | | |
| | SEQ ID NO: 82 | Cy5-TTCTGACCTGAAGGCTCTGCGCG- | 80 |
| | RNase P Probe | BHQ3 | |
| | 1.2 | | |
| | SEQ ID NO: 83 | TTCTAGCATGGTGGAGGCCAT | 500 |
| | IAV-F1.1 | | |
| | SEQ ID NO: 84 | CCGTCTGAGTTCTTCAATGGTGG | 500 |
| | IAV-R1.2 | | |
| | SEQ ID NO: 85 | YY-TCTAGGGCCCGGATTGATGCCA-BHQ2 | 200 |
| | IAV-Probe1.2 | | |
| | SEQ ID NO: 86 | AGTGGACTCAGGAAAGTGGC | 500 |
| | IBV-F2.1 | | |
| | SEQ ID NO: 87 | TCCATtTGtTGCATTGATTGAAGC | 500 |
| | IBV-R2.3 with | | |
| | LNA-Ts (t) | | |
| | SEQ ID NO: 88 | YY-TCCaAATGAAaTGGGGAATGGAAGCT- | 200 |
| | IBV-Probe2.3 | BHQ2 | |
| | with LNA-As (a) | | |

TABLE 3
Sequences of all primers and probes that were used during
optimization of SARS-CoV-2, IAV and IBV detection. The mixed bases in RdRP
sequences have been replaced by A based on 700 known SARS-CoV-2 genomes. Detection
of Human RNase P was performed according the CDC protocol, without further optimizations
and changes in the primer sequences.

| | | |
|---|---|---|
| | | Tm |
| Oligo name | Sequence (5′ - 3′) in combination with preferred label/s | (° C.) |

E gene

| | | |
|---|---|---|
| SEQ ID NO: 89 | ACAGGTACGTTAATAGTTAATAGCGT | 53.8 |
| E_Sarbeco_F1 | | |
| SEQ ID NO: 90 | CTCATTCGTTTCGGAAGAGACAGG | 57.4 |
| E_Sarbeco_F2 | | |
| SEQ ID NO: 91 | GTACTCATTCGTTTCGGAAGAGACAG | 57.0 |
| E_Sarbeco_F3 | | |
| SEQ ID NO: 92 | CGTTTCGGAAGAGACAGGTACG | 57.4 |
| E_Sarbeco_F4 | | |
| SEQ ID NO: 93 | TGTACTCATTCGTTTCGGAAGAGACA | 57.7 |
| E_Sarbeco_F5 | | |
| SEQ ID NO: 94 | TGTACTCATTCGTTTCGGAAGAGACAG | 58.1 |
| E_Sarbeco_F6 | | |
| SEQ ID NO: 95 | AtGtACTCATTCGTTTCGGAAGA | 57.8 |
| E_Sarbeco_F7 | | |
| SEQ ID NO: 96 | AtGtACTCATTCGTTTCGGAAGAC | 58.5 |
| E_Sarbeco_F8 | | |
| SEQ ID NO: 97 | ATGtACtCAtTCGTTTCGGA | 56.7 |
| E_Sarbeco_F9 | | |
| SEQ ID NO: 98 | AtGtACtCATTCGTTTCGGA | 57.0 |
| E_Sarbeco_F10 | | |
| SEQ ID NO: 99 | ATGtACTCAtTCGTTtCGGA | 57.1 |
| E_Sarbeco_F11 | | |
| SEQ ID NO: 100 | AtGTACTCATtCGTTtCGGA | 57.1 |
| E_Sarbeco_F12 | | |
| SEQ ID NO: 101 | ATGtACTCAtTCGTtTCGGA | 56.9 |
| E_Sarbeco_F13 | | |
| SEQ ID NO: 102 | ATATTGCAGCAGTACGCACACA | 57.2 |
| E_Sarbeco_R2 | | |
| SEQ ID NO: 103 | FAM/HEX/YY-ACACTAGCCATCCTTACTGCGCTTCG- | 62.3 |
| E_Sarbeco_P1 | BHQ1/BHQ2 | |
| SEQ ID NO: 104 | FAM/HEX/YY- | 60.1 |
| E_Sarbeco_P2 | TAGCGTACTTCTTTTTCTTGCTTTCGTGGT- | |
| | BHQ1/BHQ2 | |
| SEQ ID NO: 105 | FAM-CGAAGCGCAGTAAGGATGGCTAGTGT-BHQ1 | 62.3 |
| Sarbeco_P1rev | | |
| SEQ ID NO: 106 | FAM-TAGCGTACTTCTTtTTCTTGCTTTCGTGGT-BHQ1 | 61.3 |
| E_Sarbeco_P3 | | |
| SEQ ID NO: 107 | FAM-TAGCGTACTTCTtTtTCTTGCTTTCGTGGT-BHQ1 | 62.5 |
| E_Sarbeco_P4 | | |
| SEQ ID NO: 108 | HEX-TAGCGTACTTCTTtTTCTTGCTTTCGTGGT-BHQ2 | 61.3 |
| E_Sarbeco_P5 | | |

RdRP gene

| | | |
|---|---|---|
| SEQ ID NO: 109 | GTGAAATGGTCATGTGTGGCGG | 59.0 |
| RdRP_SARSr-F2 | | |
| SEQ ID NO: 110 | CGTGACAGCTTGACAAATGTTAAAAAC | 55.7 |
| RdRP_SARSr-R2 | | |
| SEQ ID NO: 111 | CGTGACAGCTTGACAAATGTtAAAAAC | 57.3 |
| RdRP_SARSr-R4 | | |
| SEQ ID NO: 112 | CGTGACAGCTTGACAAAtGTtAAAAAC | 58.8 |
| RdRP_SARSr-R5 | | |
| SEQ ID NO: 113 | FAM/HEX/YY-CAGGTGGAACCTCATCAGGAGATGC- | 61.0 |
| RdRP_SARSr-P2 | BHQ1/BHQ2 | |
| SEQ ID NO: 114 | FAM-CAGGTGGAACCT-BHQ1-CATCAGGAGATGC- | 61.0 |
| RdRP_SARSr-P3 | BHQ1 | |
| SEQ ID NO: 115 | FAM-GTGGAACCTCATCAGGAGATGCCAC-BHQ1 | 61.2 |
| RdRP_SARSr-P4 | | |
| SEQ ID NO: 116 | FAM-GTGGAACCT-BHQ1-CATCAGGAGATGCCAC- | 61.2 |
| RdRP_SARSr-P5 | BHQ1 | |
| SEQ ID NO: 117 | FAM-GGAACCTCATCAGGAGATGCCACAAC-BHQ1 | 61.2 |
| RdRP_SARSr-P6 | | |
| SEQ ID NO: 118 | FAM-GGAACCTCAT-BHQ1-CAGGAGATGCCACAAC- | 61.2 |
| RdRP_SARSr-P7 | BHQ1 | |
| SEQ ID NO: 119 | FAM/HEX/YY-TCAGGAGATGCCACAACTGCTTATGC- | 61.0 |
| RdRP_SARSr-P8 | BHQ1/BHQ2 | |
| SEQ ID NO: 120 | FAM-TCAGGAGAT-BHQ1-GCCACAACTGCTTATGC- | 61.0 |
| RdRP_SARSr-P9 | BHQ1 | |

IAV

| | | |
|---|---|---|
| SEQ ID NO: 121 | TTCTAGCATGGTGGAGGCCAT | 58.5 |
| IAV-F1.1 | | |
| SEQ ID NO: 122 | ATTTCTAGCATGGTGGAGGCC | 57.3 |
| IAV-F1.2 | | |
| SEQ ID NO: 123 | AATCCCCTGAATCCCTTTGT | 53.9 |
| IAV-F2.1 | | |
| SEQ ID NO: 124 | AaTCCCCTGAaTCCCTTTGT | 57.0 |
| IAV-F2.2 | | |
| SEQ ID NO: 125 | AAtCCCCTGAAtCCCTTTGT | 58.2 |
| IAV-F2.3 | | |
| SEQ ID NO: 126 | CGTCTGAGTTCTTCAATGGTGG | 55.7 |
| IAV-R1.1 | | |
| SEQ ID NO: 127 | CCGTCTGAGTTCTTCAATGGTGG | 57.7 |
| IAV-R1.2 | | |
| SEQ ID NO: 128 | TCTGGCATCAATCCGGGC | 58.1 |
| IAV-R2.1 | | |
| SEQ ID NO: 129 | AGGGCCCGGATTGATGCCAGA | 63.6 |
| IAV-Probe1.1 | | |
| SEQ ID NO: 130 | FAM/HEX/YY-TCTAGGGCCCGGATTGATGCCA- | 62.8 |
| IAV-Probe1.2 | BHQ1/BHQ2 | |
| SEQ ID NO: 131 | AAGAGtTCTCTGaGATCAtGAaGATC | 60.7 |
| IAV-Probe1.3 | | |

IBV

| | | |
|---|---|---|
| SEQ ID NO: 132 | AAGCAATGCCAGCATGGGAA | 58.1 |
| IBV-F1.1 | | |
| SEQ ID NO: 133 | TGaATGAaAGCAATGCCAGCA | 58.4 |
| IBV-F1.2 | | |
| SEQ ID NO: 134 | GaATGaAAGCAATGCCAGCA | 57.3 |
| IBV-F1.3 | | |
| SEQ ID NO: 135 | AGTGGACTCAGGAAAGTGGC | 57.1 |
| IBV-F2.1 | | |
| SEQ ID NO: 136 | TTtAGGATtGGCTCCCTATTTGT | 57.7 |
| IBV-F2.2 | | |
| SEQ ID NO: 137 | TTtAGGATtGGCTCCCTATTTGTG | 58.5 |
| IBV-F2.3 | | |
| SEQ ID NO: 138 | ACTGTGTTTAGGATTGGCTCCCTA | 57.7 |
| IBV-F2.4 | | |
| SEQ ID NO: 139 | GGGAGCCAATCCTAAACACAGT | 57.1 |
| IBV-R1.1 | | |
| SEQ ID NO: 140 | GaGCCAaTCCTAAACACAGT | 56.3 |
| IBV-R1.2 | | |
| SEQ ID NO: 141 | GaGCCAaTCCTaAACACAGT | 57.2 |
| IBV-R1.3 | | |
| SEQ ID NO: 142 | TCCATTTGTTGCATTGATTGAAGC | 55.0 |
| IBV-R2.1 | | |
| SEQ ID NO: 143 | TCCATTTGTTGCATTGATTGAAGCA | 56.3 |
| IBV-R2.2 | | |
| SEQ ID NO: 144 | TCCATtTGtTGCATTGATTGAAGC | 57.7 |
| IBV-R2.3 | | |
| SEQ ID NO: 145 | AGAGTGGACTCAGGAAAGTGGCC | 60.8 |
| IBV-Probe1.1 | | |
| SEQ ID NO: 146 | CAGAGTGGACTCAGGAAAGTGGCC | 61.3 |
| IBV-Probe1.2 | | |
| SEQ ID NO: 147 | TCCAAATGAAATGGGGAATGGAAGCT | 59.0 |
| IBV-Probe2.1 | | |
| SEQ ID NO: 148 | AGATCCAAATGAAATGGGGAATGGAAGCT | 60.3 |
| IBV-Probe2.2 | | |
| SEQ ID NO: 149 | FAM/HEX/YY-TCCaAATGAAaTGGGGAATGGAAGCT- | 60.8 |
| IBV-Probe2.3 | BHQ1/BHQ2 | |
| SEQ ID NO: 150 | TGCAGAGTGAATGGCACAAATAAGATCCA | 60.2 |
| IBV-Probe2.4 | | |
| SEQ ID NO: 151 | TGCCGAGTGAATGGCACAAATAAGATCC | 60.9 |
| IBV-Probe2.5 | | |
| SEQ ID NO: 152 | ACTGTGTTTAGGATTGGCTCCCTATTTGTG | 60.2 |
| IBV-Probe2.6 | | |
| SEQ ID NO: 153 | CTGtGTTTAGGATtGGCTCCCTAtT | 61.5 |
| IBV-Probe2.7 | | |

RNase P

| | | |
|---|---|---|
| SEQ ID NO: 154 | AGATTTGGACCTGCGAGCG | 57.9 |
| RNase P Forward | | |
| SEQ ID NO: 155 | GAGCGGCTGTCTCCACAAGT | 59.8 |
| RNase P Reverse | | |
| SEQ ID NO: 156 | FAM/HEX/YY/Cy5-TTCTGACCTGAAGGCTCTGCGCG- | 63.3 |
| RNase P Probe | BHQ1/2/3 | |

One-Step RT-qPCR Optimization

The optimal RT-qPCR conditions described above are the results of optimizing the thermal profile and composition of the reaction mixture. Optimal RT-qPCR conditions were determined for each kit separately and the individual optimization steps are described in Table 4. Not all alternative thermal profiles were tested in combination with each additives/alteration. In the process of the thermal profile optimization, the composition of the reaction mixture recommended by the manufacturer was used. Additives or alterations in reaction mixture composition were tested using an optimized thermal profile (marked in bold).

TABLE 4
Optimization of one-step RT-qPCR thermal profiles and reaction
mixtures.

| | | Tested additives/alterations |
|---|---|---|
| | | in reaction mixture |
| | | composition |
| | | (shown are only components, |
| | Alternative thermal | that were changed during |
| Kit | profiles tested | optimization) |
| vDetect v1. | 50° C.-10 min | |
| | 95° C.-3 min | |
| | 45x 95° C.-5 s, 58° C.-20 s | |
| | 50° C.-10 min | |
| | 95° C.-3 min | |
| | 45x 95° C.-5 s, 60° C.-20 s | |
| vDetect v2. | 50° C.-30 min | RT/RNase Block: 2 μl |
| | 95° C.-3 min | |
| | 45x 95° C.-5 s, 60° C.-20 s | |
| | 50° C.-10 min | |
| | 95° C.-3 min | |
| | 45x 95° C.-5 s, 60° C.-20 s | |
| | 50° C.-10 min | |
| | 97° C.-3 min | |
| | 45x 95° C.-5 s, 60° C.-20 s | |
| rTEST | 55° C.-10 min | 1.5x One-step Probe |
| | 95° C.-10 min | CoV Mix |
| | 45x 95° C.-15 s, 60° C.-30 | (ROX) |
| | s | |
| | 55° C.-10 min | |
| | 95° C.-10 min | |
| | 45x 95° C.-10 s, 60° C.-20 | |
| | s | |
| | 55° C.-20 min | |
| | 95° C.-10 min | |
| | 45x 95° C.-15 s, 60° C.-30 | |
| | s | |
| | 48° C.-30 min | |
| | 95° C.-10 min | |
| | 45x 95° C.-15 s, 60° C.-30 | |
| | s | |
| rTEST | 55° C.-10 min | 80 ng ET SSB |
| Allplex | 95° C.-10 min | 150 ng ETSSB |
| | 45x 95° C.-25 s, 60° C.-30 | 1.5 mM MgCl₂ |
| | s | 1.5 mM MgCl₂ + 80 ng ET |
| | 55° C.-10 min | SSB |
| | 95° C.-10 min | 200 μM dNTP |
| | 45x 95° C.-20 s, 60° C.-20 | 200 μM dNTP + 80 ng SSB |
| | s | 200 μM dNTP + 1.5 mM |
| | 55° C.-10 min | MgCl₂ |
| | 95° C.-10 min | 400 μM dNTP |
| | 45x 95° C.-15 s, 60° C.-30 | 400 μM dNTP + 1.5 mM |
| | s | MgCl₂ |
| | 55° C.-10 min | |
| | 95° C.-10 min | |
| | 45x 95 °C.-10 s, 60° C.-30 | |
| | s | |

Positive Controls

The EDX SARS-CoV-2 Standard (Exact Diagnostics) was used as a positive control for test optimization and LoD experiments. The EDX SARS-CoV-2 Standard is manufactured with synthetic RNA transcripts containing five gene targets (E, N, ORF1ab, RdRP and S Genes of SARS-CoV-2) in concentration of 200 cp/μl. The product contains genomic DNA allowing to validate testing of the entire process of a molecular assay including extraction, amplification, and detection.

“AMPLIRUNO INFLUENZA A H3 RNA CONTROL” (Vircell Microbiologists) containing the complete IAV genome, diluted to 200 cp/μl, was used as a control template for IAV assay optimization and LoD experiments. Viral RNA isolated from a MDCK cell line infected with Influenza B 17/381 was diluted to 200 cp/μl and used as a template for IBV detection. Isolation of Influenza B 17/381 was performed with QIAamp Viral RNA Mini Kit (Qiagen) according to the manufacturer's recommendations.

A synthetic matrix “SARS-CoV-2 Negative” (Exact Diagnostics) containing genomic DNA at a concentration of 75 cp/μl was used to dilute the positive control materials to desired concentrations.

The positive control PC BMC5 consists of lyophilized isolated full genomic RNA of SARS-CoV-2 virus spiked with human RNA extracted from the human cell line A549. For the purpose of determining the minimum stability of the lyophilised positive control and thus of the diagnostic kit at room temperature, three versions of the PC BMC5 were prepared: pure positive control, positive control stabilized by addition of Baker's yeast tRNA in a final concentration of 20 μg/ml and positive control stabilized by addition of salmon sperm DNA in a final concentration of 100 μg/ml. After lyophilization, the stability of the positive control stored at room temperature for 0, XYZ and 33 days was tested by RT-qPCR and was compared with non-lyophilized positive control.

The positive control PC4.01 consists of lyophilized isolated full genomic RNA of SARS-CoV-2, IAV, and IBV spiked with human RNA extracted from the human cell line A549 and stabilizer (Baker's yeast tRNA or salmon sperm DNA). The positive control PC BMC5 consists of lyophilized isolated full genomic RNA of SARS-CoV-2 virus spiked with human RNA extracted from the human cell line A549. PC BMC5 and PC4.01 positive controls were diluted to show a Ct values in the range of 28-35.

Analytical Sensitivity (Limit of Detection)

Evaluation of analytical sensitivity (limit of detection) was performed using 8 replicates over multiple concentrations, and 24 additional replicates were performed at concentrations spanning the level with 95% detection. In case of vDetect v1, the dilutions were prepared by serial dilutions of the stock standard, resulting in samples with concentrations of 40 copies/μl (=200 copies/reaction), 8 copies/μl (=40 copies/reaction), 1.6 copies/μl (=8 copies/reaction), and 0.25 copies/μl (=1.25 copies/reaction).

The dilutions of all the other kits were prepared by serial dilutions of the stock standard, resulting in samples with concentrations of 8 copies/μl (=40 copies/reaction), 2 copies/μl (=10 copies/reaction), 0.8 copies/μl (=4 copies/reaction), and 0.4 copies/μl (=2 copies/reaction) that were used in the analytical sensitivity test. A synthetic matrix “SARS-CoV-2 Negative” (Exact Diagnostics) containing genomic DNA at a concentration of 75,000 copies/ml was used to dilute the control material.

Test Specificity

Evaluation of specificity (potential cross-reactivity to other coronaviruses and respiratory viruses) was performed using the control material “Coronavirus RNA specificity panel” (EVAg, European Virus Archive—Global), which contains RNA viruses HCoV-229E, HCoVOC43, HCoV-N163, SARS-CoV HKU39849, and MERSCoV, each in a separate tube. The EDX SARS-CoV-2 Standard (Exact Diagnostics) was used as a reference material for this test. A set of respiratory viruses (Vircell) containing RNA of Influenza A H1N1, Novel Influenza A H1N1, Influenza A H3N2, Influenza A H5N1, Novel Influenza B, Human parainfluenza, Respiratory syncytial virus and Human rhinovirus, each provided in a separate tube, were used to assess cross-reactivity to respiratory viruses. All assays were performed in triplicate for each of the indicated viruses.

Clinical Evaluation

Evaluation of the clinical performance for SARS-CoV-2 was performed for the E gene screening test, the confirmatory test for the RdRP gene as well as for human RNase P. Regarding IAV and IBV, the evaluation was performed for IAV PB1 gene and IBV PA gene. For SARS-CoV-2, the evaluation was performed on a selected set of 38 positive and 54 negative clinical samples of patients IAV and IAB, the evaluation was performed on a selected set of 52 and 37 clinical samples of patients with IAV and IBV, respectively. One sample was negative for both, IAV and IBV. RNA was extracted from nasopharyngeal samples using RNAdvance Viral Kit and the Biomek i5 Automated Workstation (Beckman Coulter). Samples were exposed to one freeze-thaw cycle before RNA extraction. Testing of this selected set of samples was performed with blinded samples. All samples used in the validation were confirmed by a reference method used for routine testing by regional public health authorities of the Slovak Republic.

Results

Redesign and Optimization of Charité SARS-CoV-2 E and RdRP Primer/Probe Sets

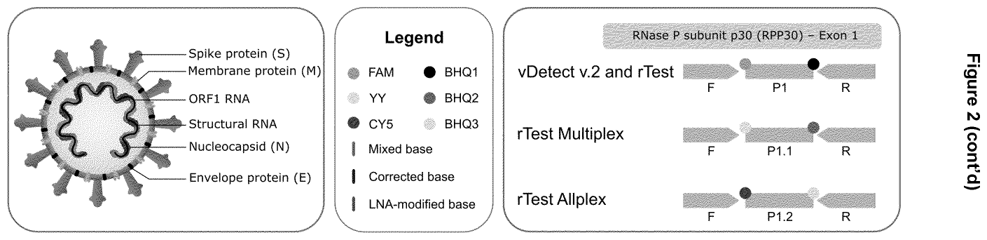

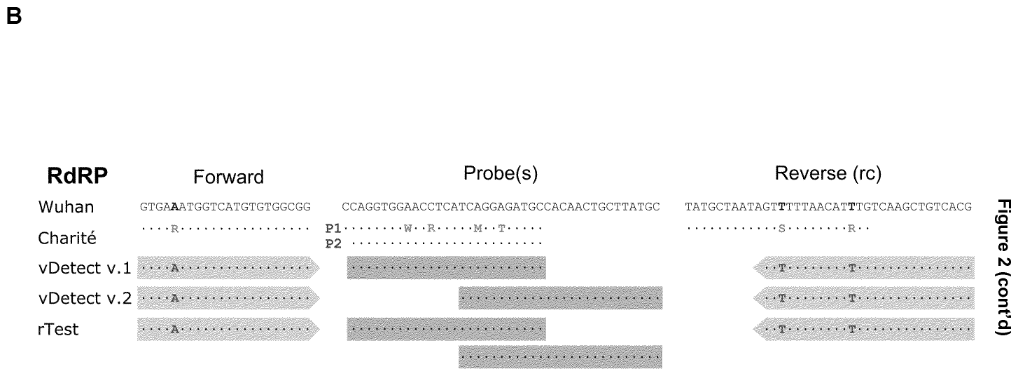

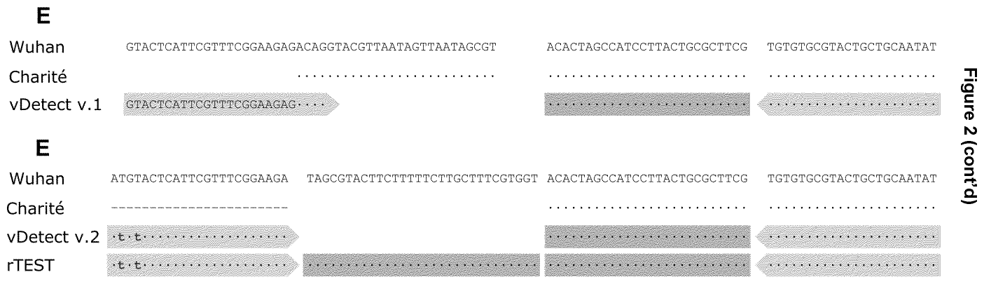

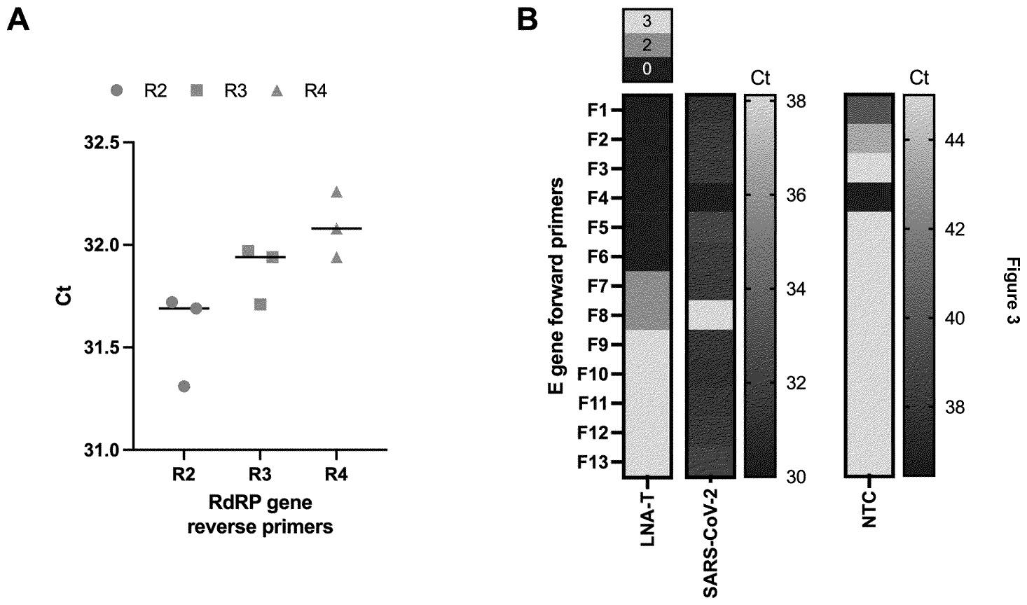

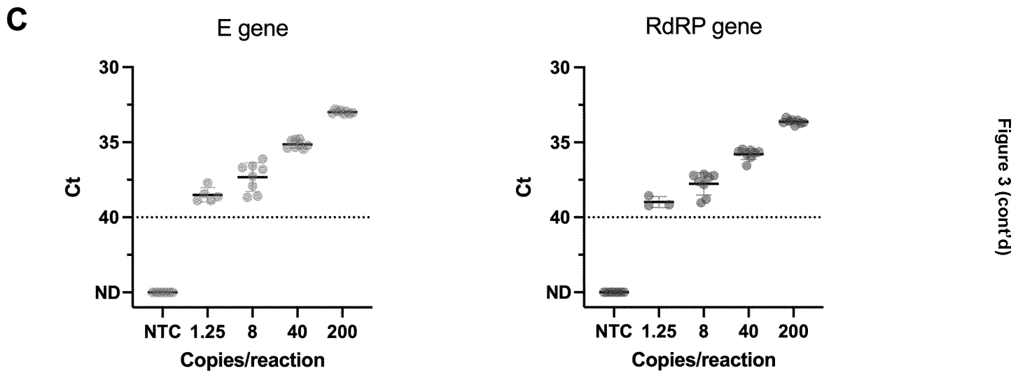

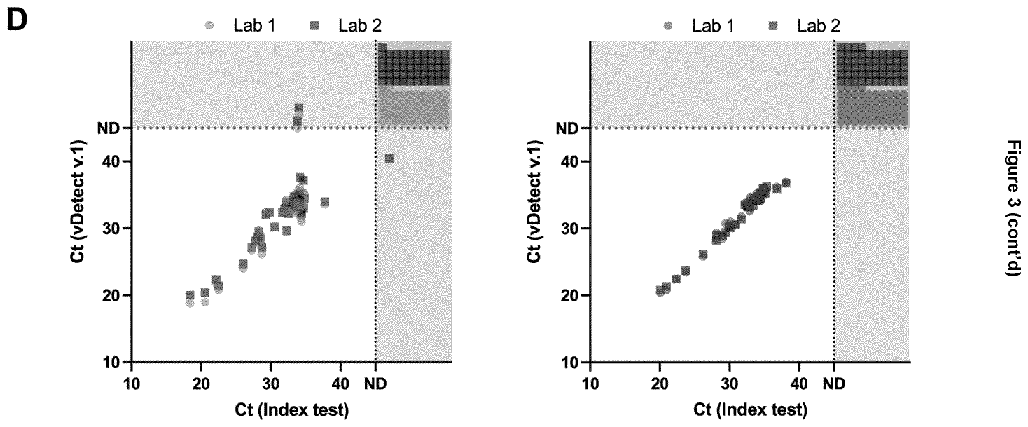

As a starting point for our SARS-CoV-2 RT-qPCR test, we used the E and RdRP primer/probe sets developed by the Charité Institute of Virology (Berlin) as a backbone for our test development. We aligned 700 SARS-CoV-2 sequences against the Wuhan reference genome and used the 95% consensus sequence to verify the specificity of the Sarbecco E gene and RdRP gene primer/probe set. Since the alignments identified several degenerate bases placed in the RdRP forward and reverse primers that resulted in mismatches to the consensus sequence, we replaced these degenerate bases with the appropriate complementary bases (FIG. 2). An in silico analysis to assess melting temperatures and the potential formation of dimers and secondary structures also found that the RdRP reverse primer had a significantly lower annealing temperature relative to the forward primer (8.5° C. Tm difference). This deficiency in primer design could reduce the efficiency and sensitivity of the RdRP assay. To address this, we designed three primers located downstream of the Charité reverse primer (R1), lengthened the primers by one base, and incorporated LNA-modified nucleotides to stabilize the 3′-end and increase the melting temperatures of the forward primers (Table 2 and Table 3). While all primer sets amplified the SARS-CoV-2 positive control, the unmodified primer (R2) performed better than the LNA-modified reverse primers (R3 and R4; B; FIG. 3A). This R2 reverse primer also had a closer melting temperature to the forward primer (3.3° C. Tm difference) so we selected R2 as the reverse primer for further experiments.

At the beginning of the pandemic, labs worldwide implemented RT-qPCR protocols to detect SARS-CoV-2 that were largely based on WHO-approved protocols, including the Charité assay. Because of the excessive demand for primers/probes and synthetic positive controls, many labs reported receiving primers/probes that were contaminated with synthetic positive controls for the E gene (Fischer C et al. Variable sensitivity in molecular detection of SARS-CoV-2 in European Expert Laboratories: External Quality Assessment, June-July 2020. J Clin Microbiol. 2020 Dec. 9; Huggett J F, et al. Cautionary Note on Contamination of Reagents Used for Molecular Detection of SARS-CoV-2. Clinical Chemistry. 2020 Nov. 1; 66(11):1369-72; Mögling R, et al. Delayed Laboratory Response to COVID-19 Caused by Molecular Diagnostic Contamination—Volume 26, Number 8-August 2020—Emerging Infectious Diseases journal—CDC; Wernike K, et al., 2020, Pitfalls in SARS-CoV-2 PCR diagnostics. Transboundary and Emerging Diseases 14 Jun. 2020). We also experienced contaminated primer/probe sets with synthetic E gene templates so we redesigned the E gene forward primer by shifting its location to a more upstream location to create a primer/probe set that would not amplify the most common SARS-CoV-2 E gene synthetic controls (FIG. 2). Since the E gene is only 228 bases, we also incorporated varying numbers of LNA nucleotides enabling the design of shorter forward primers that do not overlap with the original Charité forward primer, while maintaining sufficient melting temperatures. While all these primers amplified SARS-CoV-2 template equally well, except for F8, some of the forward primers that overlapped with the original Charité forward primer continued to display amplification in the absence of template (e.g., F1, F2, and F4; FIG. 3B). Based on these data as well as the fluorescent intensity generated by each primer/probe set, we selected F3 as the optimal forward primer (FIG. 2).

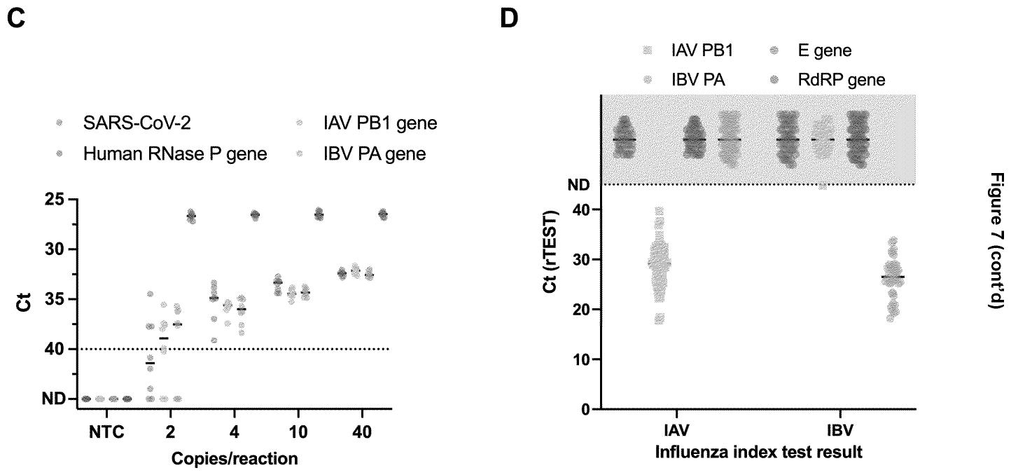

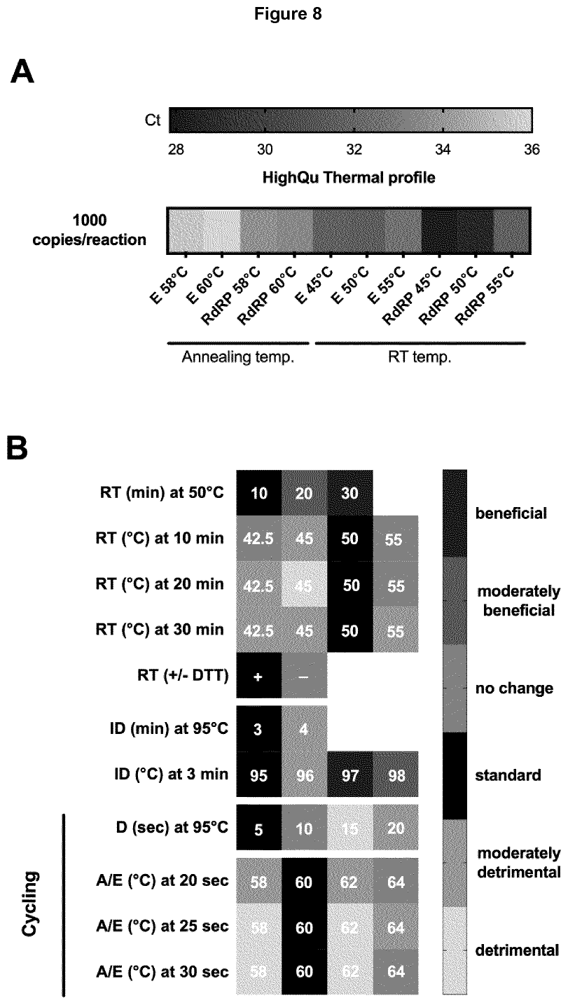

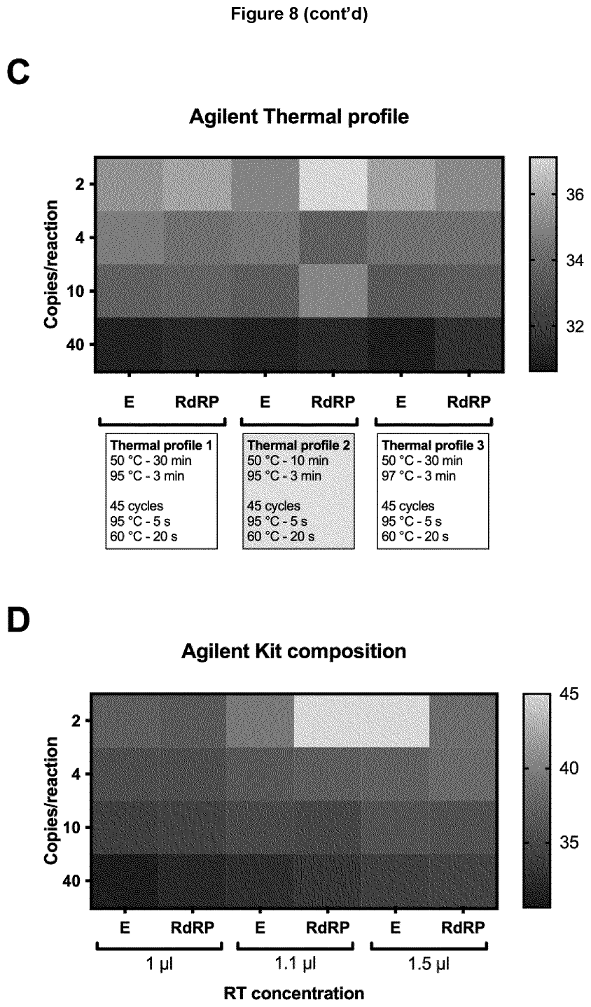

We also investigated the optimal reverse transcription (RT) and annealing temperatures. Temperatures deviating above or below the standard RT (50° C.) were either detrimental or had no effect on amplification for either E or RdRP assays. Although a lower annealing temperature (58° C.) caused minor improvement in E gene detection, we opted to maintain the manufacturer's recommendations (FIG. 8A). With this final primer/probe set, called vDetect v.1, we determined the LoD for both E and RdRP assays to be 8 copies/reaction (FIG. 3C). To assess the clinical performance of this revamped version of the Charité protocol, called vDetect v.1 COVI D-19 RT-qPCR test, two

independent labs conducted the test on 92 clinical samples and compared the results to an index RT-qPCR test for SARS-CoV-2 used for routine screening by public health officials. Similar to the original Charité protocol, the test workflow consisted of an initial screening test for detection of the E gene followed by a confirmation test for detection of the RdRP gene. Our vDetect v.1 test correctly identified all positive (38/38) and negative (52) samples, and even identified two false positive samples that were incorrectly classified by the E gene assay of the reference method (FIG. 3D; Table 5).

TABLE 5
Clinical performance of vDetect v.1 COVID-19 RT-qPCR test.

| | | | vDetect COVID- | | vDetect COVID- | |
|---|---|---|---|---|---|---|
| | Reference | | 19 RT-qPCR kit | | 19 RT-qPCR kit | |
| | method | | laboratory 1 | | laboratory 2 | |
| | E | RdRP | E | RdRP | E | RdRP |
| | gene | gene | gene | gene | gene | gene |
| Number of correctly | 38 | 38 | 38 | 38 | 38 | 38 |
| identified positive | | | | | | |
| samples | | | | | | |
| Number of false | 2 | 0 | 0 | 0 | 0 | 0 |
| positive samples | | | | | | |
| Number of correctly | 52 | 54 | 54 | 54 | 54 | 54 |
| identified negative | | | | | | |
| samples | | | | | | |
| Number of false | 0 | 0 | 0 | 0 | 0 | 0 |
| negative samples | | | | | | |

Since the LoD of vDetect v.1 was slightly less sensitive than the reported LoDs for E and RdRP (5.2 and 3.8 copies/reaction, respectively) in the original Charité protocol, we switched to the Agilent Brilliant III Ultra-Fast QRT-PCR Master Mix, which in our internal testing yielded superior results, and conducted a thorough optimization of reaction parameters and reaction composition. Initially, we assessed the performance of a variety of parameters by analyzing PCR products using gel electrophoresis and found that extending the RT reaction to 30 min and increasing the initial denaturation temperature to 97° C. were beneficial (FIG. 8B). To verify these beneficial modifications, we tested three thermal profile variants using RT-qPCR and determined that extending the RT reaction to 30 min improved detection of RdRP and that elevating the initial denaturation temperature provided no additional benefit (FIG. 8C). Using RT-qPCR, we also observed that increasing the concentration of reverse transcriptase in the reaction either had no effect or was detrimental (FIG. 8D). Altogether, we decided to eliminate the use of DTT in the RT step and increased the RT time to 30 min.

We also modified the oligonucleotides used in the RdRP and E gene assays. First, we replaced the RdRP probe (P2) with a new TaqMan hydrolysis probe (P8) that resulted in a substantial increase in fluorescent intensity (FIG. 2). Also, after experiencing sporadic amplification of NTCs with our modified E gene assay that was due to contamination with synthetic E gene positive control, we replaced the E gene forward primer (F3) with a shorter LNA-modified forward primer (F3) that did not overlap with the original forward primer and therefore eliminated amplification of the synthetic positive control. With this new mastermix with optimized reaction parameters and modified RdRP and E gene primer/probe sets, we incorporated the US CDC human RNase P primer/probe as an internal control for RNA extraction and assay performance. With this new version, vDetect v.2, we observed an improvement in sensitivity with both E and RdRP assays consistently detecting down to only 2 copies of viral RNA per reaction (FIG. 8E).

Optimization of a Room-Temperature Stable SARS-CoV-2 RT-qPCR Assay

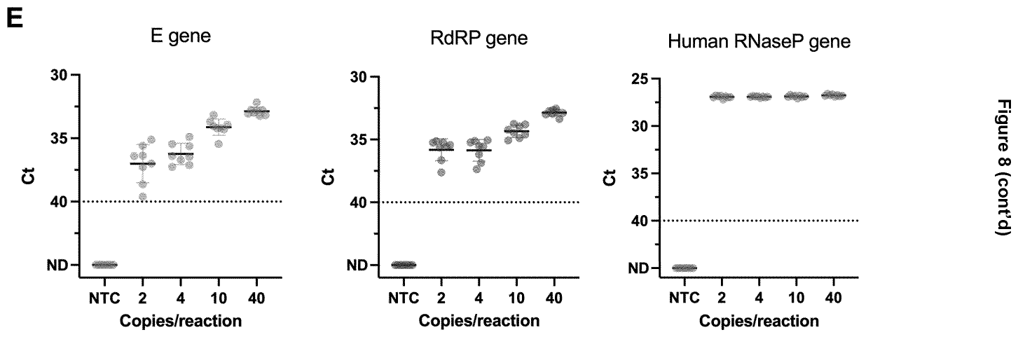

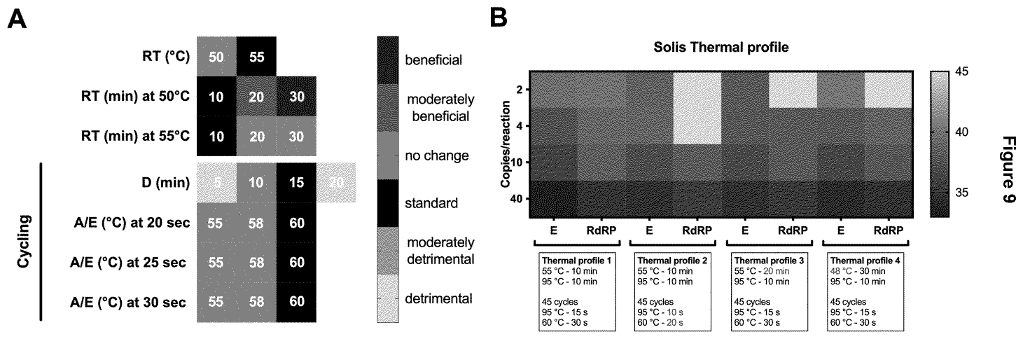

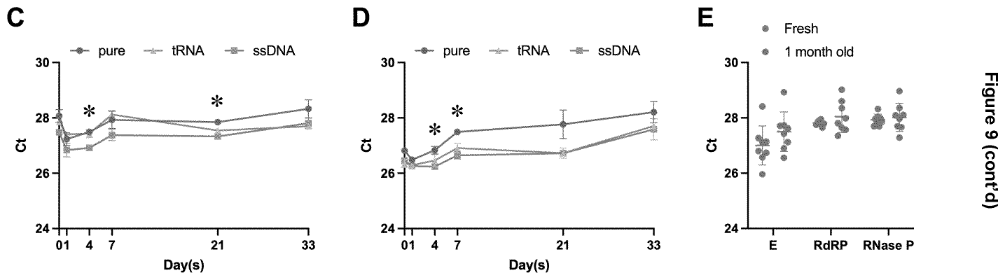

A major limitation of the majority of SARS-CoV-2 RT-qPCR assays is the requirement to ship and store reaction components at low temperatures (−20°). To address this disadvantage, we optimized our assay to be compatible with a room-temperature stable master mix (SOLIScript® 1-step CoV Kit; SOLIS BioDyne), lyophilized primer/probe mixes and positive controls, and conducted stability tests over the positive control and whole kit over a one-month period. Using both gel electrophoresis of PCR products and real time RT-qPCR, we found most modifications to the standard thermal cycling procedure produced no change or were detrimental (FIGS. 9A and B), suggesting the standard reaction parameters were optimal for our assays. Since SOLIS BioDyne already verified the room temperature stability of the master mix, we focused our efforts on stabilizing and lyophilizing our positive control (PC BMC 5). We reasoned that the main source of RNA degradation at room temperature is due to nuclease activity; therefore, we spiked our positive control with either baker's yeast tRNA or salmon sperm DNA (ssDNA) to act as carriers and decoys for nucleases and then lyophilized the positive control and tested its stability over a month period. Although only ssDNA statistically improved the stability of the lyophilized positive control over a one-month period (FIG. 9C-D), we decided to spike our positive control with both carriers for maximal protection. Leaving the entire kit (master mix, lyophilized primer/probe sets and positive control) at room temperature for one month and comparing performance to a freshly prepared kit demonstrated consistent performance for all assay targets for at least one month at room temperature (FIG. 9E).

Dual Probes Enhance Specificity and Increase Fluorescent Signal

Although coronaviruses such as SARS-CoV-2 display reduced mutation rates relative to other RNA-based viruses, there is considerable evidence that emerging SARS-CoV-2 mutations can lead to increased transmissibility, virulence, and escape from immune responses. If mutations occur in diagnostic targets of RT-qPCR assays they can lead to reduced binding efficiency of primers and probes and consequently reduced sensitivity and even failed tests (Khan K A, Cheung P. Presence of mismatches between diagnostic PCR assays and coronavirus SARS-CoV-2 genome. Royal Society Open Science. 7(6):200636). To address this issue, we designed a series of additional hydrolysis probes for both the E and RdRP assays that contain the same fluorescent reporter dyes as the first probe, essentially making the assays more robust to potential mutations in complementary sequences. In parallel, we also tested probes containing a second BHQ-1 quencher located internally, in order to reduce background fluorescence and increase the dynamic range of the fluorogenic probes.

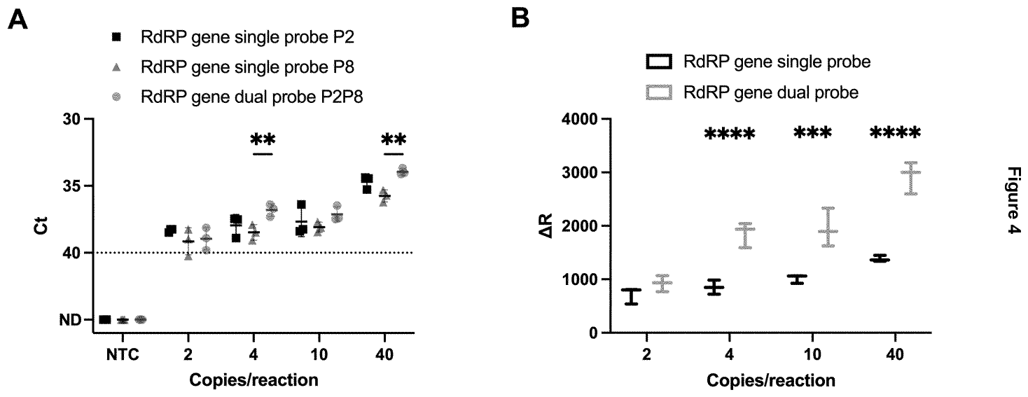

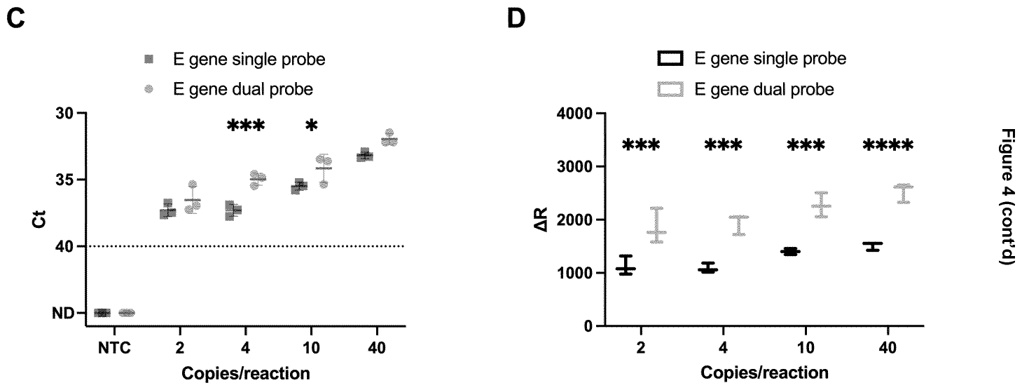

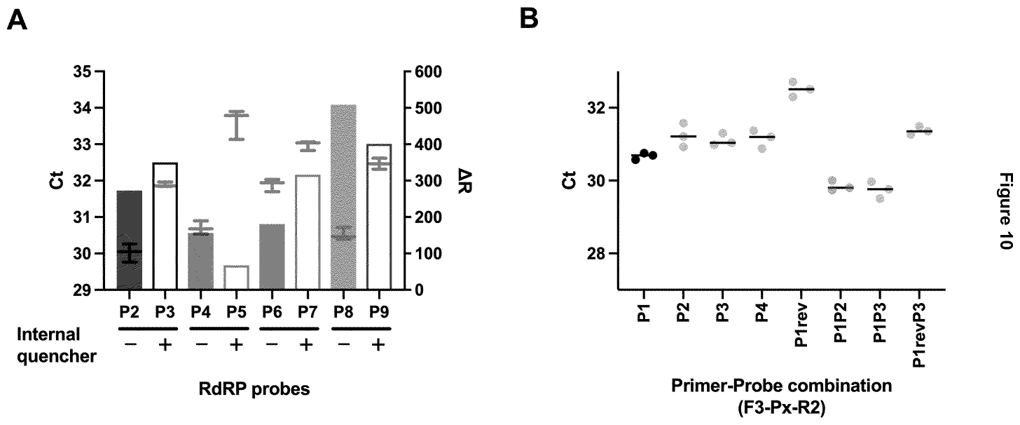

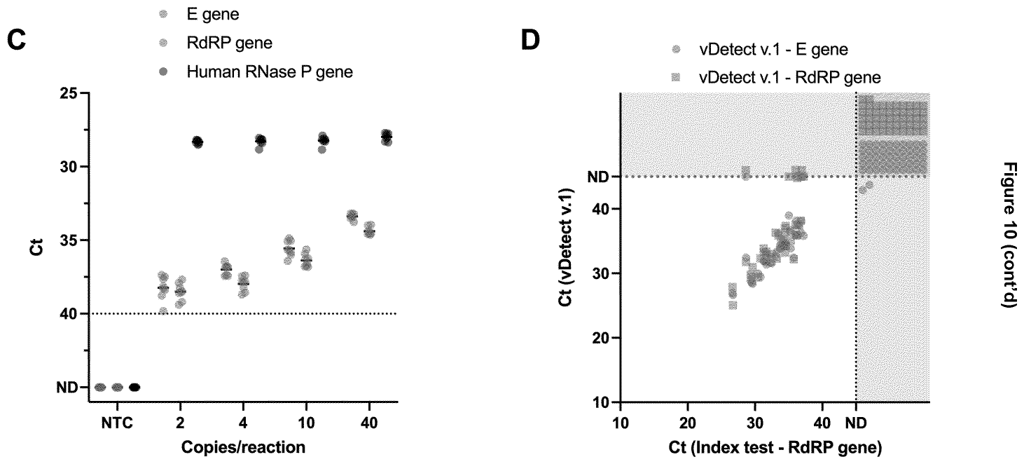

We screened a variety of RdRP probes with or without an internal quencher and surprisingly found that the internal quencher reduced sensitivity (i.e., increased Ct) and had variable effects on fluorescent intensity (FIG. 10A). The best performing probe (P8, lowest Ct and highest fluorescence (ΔR)) when included in the same reaction as our original probe (P2) increased the sensitivity of the reaction (FIG. 4A) and drastically improved the dynamic range of the assay (FIG. 4B). It is noteworthy, that P2 and P8 slightly overlap on the same strand yet still produce a significant increase in sensitivity and fluorescence intensity, suggesting little, if any, interference between these overlapping probes.

For the E gene assay, we designed an additional probe (P2) downstream of the original probe P1 with two LNA-modified variants (P3 and P4) as well as a probe (reverse complement of P1, P1rev), that would bind to the reverse strand located downstream of the reverse primer. LNA modified probes showed equivalent sensitivity to an unmodified probe (FIG. 10B). While a second probe complementary to the opposite strand was detrimental to sensitivity in both single and dual probe reactions (FIG. 10B), inclusion of a second, non-overlapping probe in tandem with the original probe enhanced sensitivity of the assay (FIG. 10B). Indeed, dual-versus single-probe reactions increased sensitivity (FIG. 4C) and dynamic range (FIG. 4D).

With our optimized room-temperature stable master mix and dual-probe assays for SARS-CoV-2 E and RdRP genes, we assessed the LoD of this new test (called rTEST COVID-19 qPCR) and confirmed an LoD of 2 copies/reaction (FIG. 10C). We thawed and re-extracted RNA from a panel of 92 clinical samples and reran the index test to compare with the original testing results. The index test, even though it was the same method as the original test (vDetect COVID-19 qPCR v2 kit), failed to identify three positive samples with the E gene assay and seven positive samples with the RdRP assay likely due to loss of RNA during the freeze/thaw process and RNA extraction (FIG. 10D). Therefore, we decided to evaluate the clinical performance of the rTEST COVID-19 qPCR kit using the most recent testing results. The analysis showed excellent concordance with the index test, with both E and RdRP assays of the rTEST COVI D-19 qPCR kit correctly identifying all negative (54/54, 100% diagnostic specificity) and positive (38/38, 100% diagnostic sensitivity) samples, including the samples that were positive in the original reference test, but negative in the retest of RNA extracted from thawed samples (FIGS. 4E and F).

Multiplexing E and RdRP Gene Assays to Streamline Testing Workflow

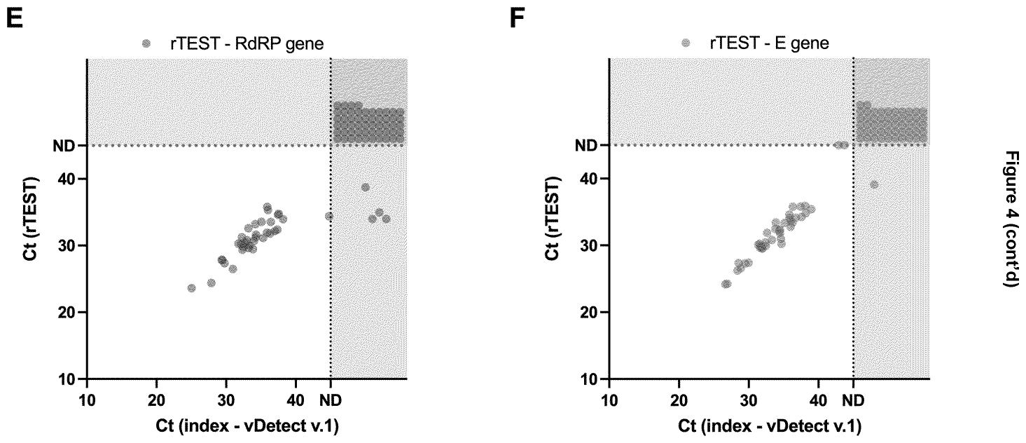

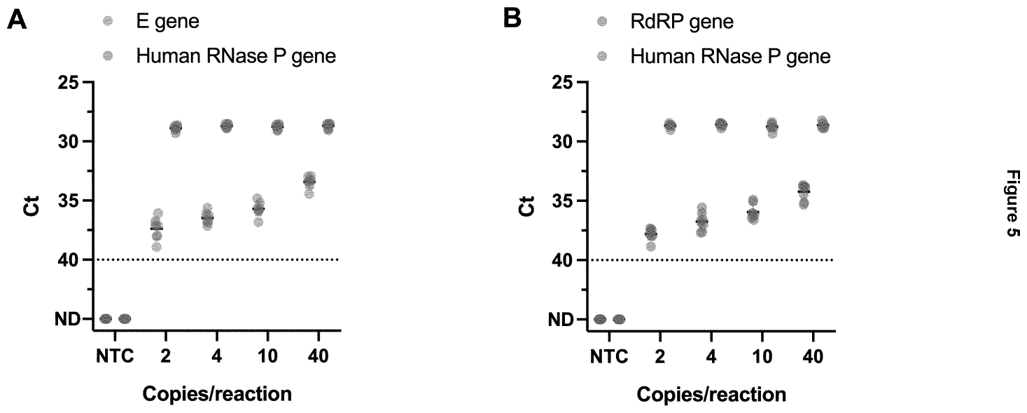

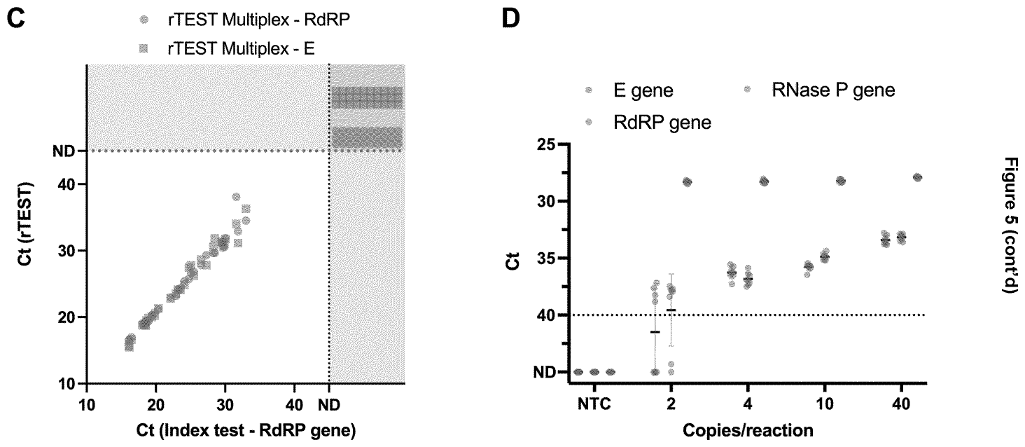

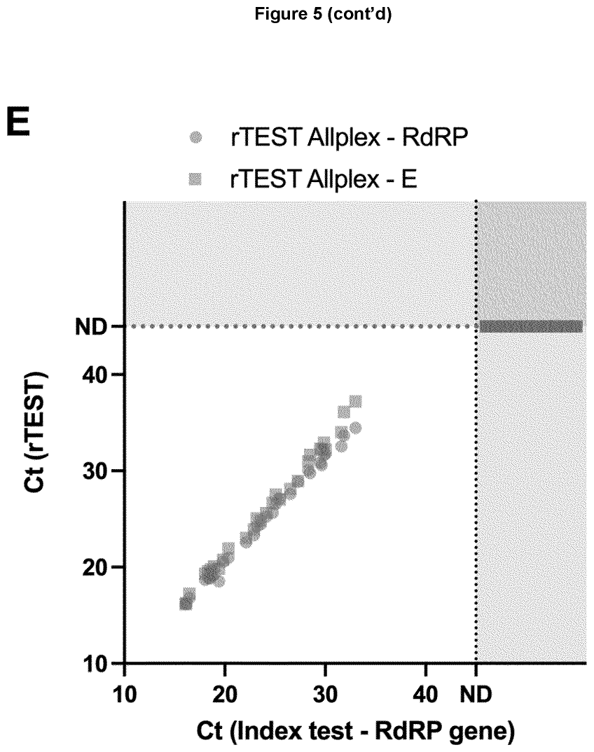

Like the original Charité protocol, the workflow of our optimized test consisted of an initial screening test using the E gene, a second confirmation test for the RdRP gene, and, in parallel, a third assay for the human RNase P internal control. The shortcomings of this lengthy workflow are counterproductive given diagnostic labs may face significant backlogs in testing. To address this limitation and enable rapid, high throughput testing, we attempted to streamline our test by multiplexing assay targets into a single reaction. We first multiplexed each of the E and RdRP gene assays (FAM dyes) with human RNase P (HEX dye). Given that two primer/probe sets were competing for a limited pool of reagents in a single reaction, we reduced the concentration of primers and probe for the more abundant RNase P assay to ensure this reaction would plateau before consuming all the reagents. We determined that that this primer limited multiplexed assay did not change the LoD (FIGS. 5A and B). Indeed, this multiplexed test (called rTEST Covid-19 qPCR Multiplex kit), like its singleplexed counterpart, showed an impressive 100% diagnostic specificity (30/30 negative samples) and sensitivity (30/30 positive samples; FIG. 5C).

Next, we multiplexed all three targets in a single reaction and further reduced the RNase P primers/probe concentrations by 50%. The limit of detection of this triplexed assay was nearly as sensitive as the singleplex and duplexed versions, detecting 100% of replicates at 4 copies/reaction (FIG. 5D). This triplexed version (called rTEST Covid-19 qPCR Allplex kit) also correctly identified all negative (30/30) and SARS-CoV-2 positive (30/30) samples during a clinical performance evaluation (FIG. 5E).

Differentiation of SARS-CoV-2 from Influenza A and B

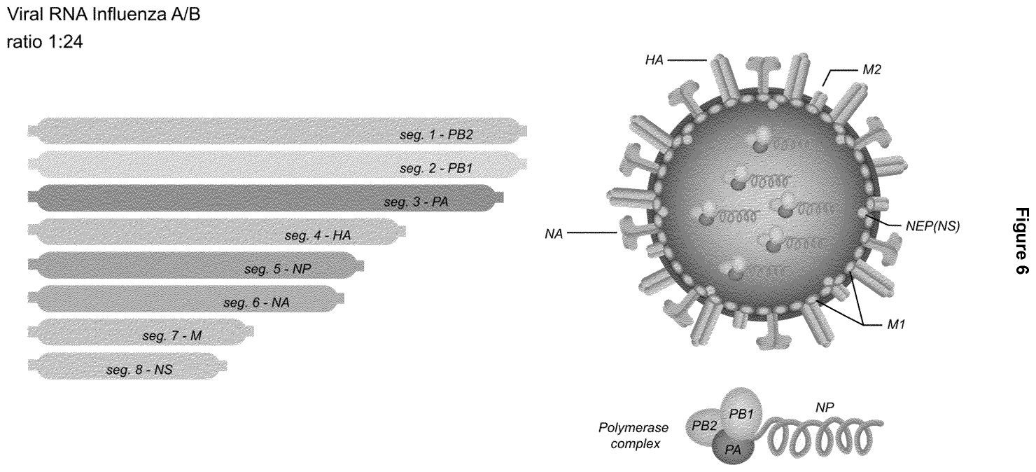

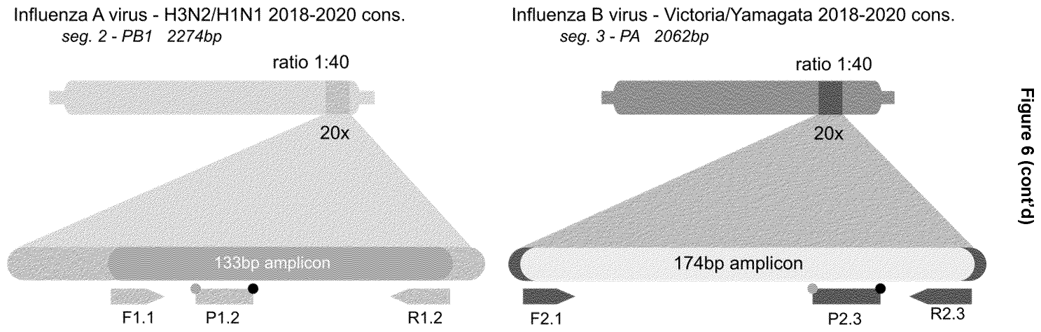

Since other respiratory pathogens such as seasonal influenza produce symptoms that overlap with SARS-CoV-2, it is important to have molecular diagnostics that can effectively differentiate between the two respiratory viruses. To develop an RT-qPCR assay that could distinguish between SARS-CoV-2 and the influenza A and B, we conducted an extensive bioinformatic analysis of over 27,000 influenza A (H1N1 and H3N2) and over 8,000 influenza B (Victoria and Yamagata) sequences deposited in GISAID from 1.1.2018 to 24.6.2020. These sequence alignments revealed several highly conserved areas with minimal amounts of mixed bases in the PB1 segment of IAV and PA segment of IBV where we designed primers/probes to target (FIG. 6).

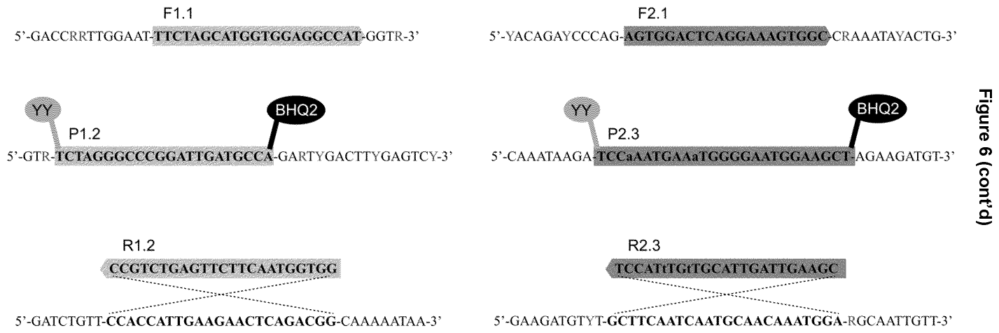

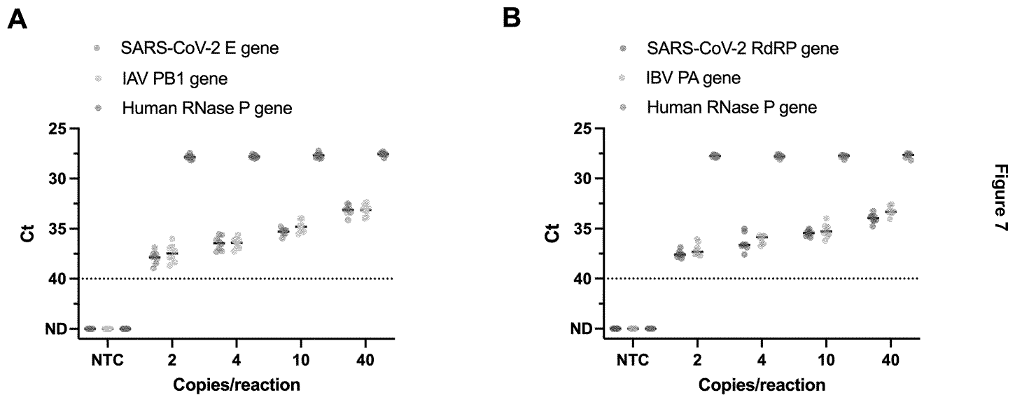

After screening a series of primers and probes, we multiplexed the optimal primer/probes sets for IAV and IBV with both SARS-CoV-2 E and RdRP genes (both labeled with FAM) and determined the analytical sensitivity. We tested two multiplexed formats: 1) the first format allows the differentiation SARS-CoV-2, IAV, and IBV and consists of two multiplexed assays containing either SARS-CoV-2 E gene multiplexed with IAV PB1 gene and RNase P or SARS-CoV-2 RdRP gene multiplexed with IBV PA gene and RNase P; and 2) the second format allows for the differentiation of SARS-CoV-2 and influenza (but not the distinction between IAV and IBV), which consists of a single multiplexed reaction containing both SARS-CoV-2 E and RdRP genes (both labelled with FAM), IAV PB1 and IBV PA genes (both labelled with YY), and RNase P (Cy5). The first format with two multiplexed reactions yielded exceptional sensitivity with all multiplexed targets detecting every replicate at only 2 copies/reaction (FIG. 7A, B); while the second format with one multiplexed reaction had a slightly higher limit of detection of 4 copies/reaction (FIG. 7C).

We assessed the clinical performance of this test, called rTEST COVID-19/FLU qPCR kit, on a selected set of 52 and 37 clinical samples of patients diagnosed with IAV and IBV, respectively, by a reference method used for routine testing by regional public health authorities of the Slovak Republic. Both the IAV PB1 and IBV PA gene assays correctly identified all positive samples (IAV PB1=52/52; IBV PA=37/37; FIG. 7D). One sample confirmed as IBV showed late amplification (Ct=44.77) with the IAV assay; however, this would be deemed as negative in clinical practice. Importantly, there was no cross reactivity between SARS-CoV-2 and either IAV or IBV samples demonstrating that the test accurately differentiates between all three viruses.

Discussion

In this example, we improved upon the original Charité SARS-CoV-2 RT-qPCR protocol by correcting mismatched bases, normalizing primer melting temperatures by using LNA-modified nucleotides and incorporating a human RNase P internal control to assess RNA extraction, RNA integrity, and assay performance. Our revamped SARS-CoV-2 assays also contain technological novelties such as dual probes to enhance specificity and sensitivity, a room-temperature stable master mix, and primer limited multiplexed assays that enable higher throughput testing while maintaining exceptional sensitivity. To aid in differentiating SARS-CoV-2 from other respiratory pathogens that have overlapping symptomatology, we multiplexed our SARS-CoV-2 assays with primer/probe sets targeting the most common seasonal influenzas.

Redesigned and Revamped RdRP and E Gene Primer/Probe Assays

Being one of the first RT-qPCR tests for SARS-CoV-2 to be published and approved by the WHO, the Charité protocol was developed without access to SARS-CoV-2 isolates or clinical specimens as well as a paucity of genomic sequences; therefore, the assay design relied on genetic sequences from closely related SARS-CoV and bat-related coronaviruses, which resulted in the placement of several degenerate bases in the RdRP primers and probe. Our bioinformatics analysis of SARS-CoV-2 genomes revealed these degenerate bases in the RdRP forward and reverse primers resulted in mismatched bases, which could be responsible for the reduced sensitivity of the RdRP assay. Another problem with the original Charité RdRP assay stems from the low melting temperature of the reverse primer. This difference in melting temperatures between the forward and reverse primers (8.5° C. Tm difference) can result in altered patterns of annealing and consequently reduced efficiency and sensitivity. This deficiency in primer design, as pointed out by the authors of the Charité protocol (Corman VM, Drosten C. Authors' response: SARS-CoV-2 detection by real-time RT-PCR. Euro Surveill. 2020 May 28), is the more likely culprit responsible for the reduced sensitivity of the RdRP assay, since PCR is generally tolerant of mismatches that occur in the middle and 5′-end of primers (as is the case here). Although we did not conduct experiments to determine the root cause of the suboptimal RdRP assay, we found that correcting the mismatched bases in both forward and reverse primers and redesigning the reverse primer to ensure a higher Tm remedied the performance issues with this assay and resulted in comparable performance to the E gene assay.

Due to reports of commercially supplied primers/probes being contaminated with synthetic positive controls for the E gene, we also redesigned the forward primer of the E gene so that it would not amplify the most common synthetic positive controls. This presented challenges because the small size of the E gene and AT-rich nucleotide content provides few choices for designing full length primers with optimal annealing temperatures. To circumvent these design limitations, we incorporated LNA-modified thymine bases into the 5′-end of the forward primer, which allowed us to shorten the length of the primer to eliminate any overlap with the Charité E gene forward primer (and consequently E gene synthetic positive controls), while still maintaining the optimal duplex annealing temperature. This new E gene forward primer design offers an innovative solution to eliminate potential issues related to contamination with synthetic positive control without having to develop an assay to a completely new gene target.

Dual Probes Increase Sensitivity and Specificity of SARS-CoV-2 RT-qPCR Assays

Based on the observation that introducing a second TaqMan hydrolysis probe into the RT-qPCR reaction, labelled with the same reporter dye, and placed either in tandem or opposite the first probe, can result in an additive increase in fluorescent intensity and can even enhance the sensitivity of the assay, we observed that a dual probe when hybridized in tandem with the original probe roughly doubled the fluorescent intensity and increased sensitivity by reducing the average Ct value at a given copy number per reaction. However, it was observed that a second hydrolysis probe placed on the opposite strand of the first probe was detrimental to sensitivity. Interestingly, the dual probes used in our RdRP assay overlap, yet still provide additive gains in fluorescent intensity and enhanced sensitivity. The novel finding that overlapping dual probes provide similar benefits to dual probes that hybridize in tandem, affords users with additional flexibility to design dual probe assays, especially when targeting difficult templates such as short templates and those containing mixed bases (e.g., viruses) or suboptimal nucleotide content (e.g., AT rich, low complexity sequences).

A pertinent benefit of using dual probes is the inherent increase in specificity of the assay. This is particularly important when developing RT-qPCR assays for detection of viruses that have a natural propensity to mutate. Mutations in the viral genome that result in mismatches in primer- or probe-binding regions can be detrimental to the performance of an assay and are part of the rationale for public health bodies to recommend multi-gene target assays for detection of SARS-CoV-2. Indeed, it is known that SARS-CoV-2 mutations can severely affect the performance of RT-qPCR assays, and that the accumulation of mutations over time and geographical location can exacerbate this problem. One example of this phenomena involves the emergence of the B.1.1.7 lineage first discovered in the UK. This variant was first identified because it contains a deletion in the spike gene that caused a rising number of RT-qPCR assays to fail—so-called spike gene target failures. By utilizing an additional dual hydrolysis probe, our SARS-CoV-2 assays contain an additional layer of specificity such that any potential mutation that results in a mismatch in one probe binding region is compensated by the other probe.

Differentiating SARS-CoV-2 from Influenza A and B

The circulation of other respiratory pathogens provides a challenging scenario for physicians in correctly distinguishing individuals infected with SARS-CoV-2 from those infected with other pathogens such as influenza because they often have overlapping symptomatology. This problem is exaggerated by reports of people being co-infected with both SARS-CoV-2 and influenza, suggesting that testing positive for another respiratory pathogen does not preclude the absence of a SARS-CoV-2 infection. Therefore, there is a need for diagnostic tools to accurately differentiate SARS-CoV-2 from other respiratory pathogens especially seasonal pathogens like influenza. To address this challenge, we conducted an extensive bioinformatics analysis of over 35,000 influenza A and B sequences emphasizing sequences arising in the past two years to ensure high representation of recent cases. This analysis identified highly conserved targets in the PB1 (IAV) and PB (IBV) segments that are ideal targets for RT-qPCR primers and probes. We also multiplexed these assays with our SARS-CoV-2 E and RdRP assays to create two reaction formats that provide unique benefits depending on the required throughput and necessity to distinguish between IAV and IBV.

### CONCLUSION

Early in the SARS-CoV-2 pandemic, several RT-qPCR protocols were published by reference laboratories and public health bodies, enabling countries to quickly setup diagnostic workflows necessary to identify the novel coronavirus. While these protocols provided unquestionable benefits and formed the basis for many commercial RT-qPCR tests, several issues emerged regarding sensitivity and specificity. This paper outlined the development of a series of RT-qPCR tests based on the protocol developed by the Charité Institute of Virology. We remedied some of the deficiencies of this original assay related to mismatches in the primers and suboptimal annealing temperatures, and made significant improvements to increase the sensitivity, specificity, throughput, and functionality. We incorporated dual probe technology to boost fluorescence signal and sensitivity, while also offering an extra layer of protection against mismatches produced by mutations in probe-binding regions. Our multiplexed assays, which also contain an RNase P internal control, drastically reduce hands-on-time and conserve laboratory resources without sacrificing sensitivity. Some of the tests contain a room temperature-stable master mix with lyophilized primers/probes stabilized with decoy nucleic acids placing them among only a few RT-qPCR tests that do not require cold chain shipping and storage. Moreover, we multiplexed these SARS-CoV-2 assays with influenza A and B assays to facilitate rapid differentiation of these respiratory pathogens that pose challenges for healthcare practitioners to identify. These novel, room temperature-stable RT-qPCR tests can provide users with a powerful tool to detect SARS-CoV-2 rapidly and accurately in the next phase of the pandemic.

One skilled in the art would readily appreciate that the present invention is well adapted to carry out the objects and obtain the ends and advantages mentioned, as well as those inherent therein. Further, it will be readily apparent to one skilled in the art that varying substitutions and modifications may be made to the invention disclosed herein without departing from the scope and spirit of the invention. The compositions, methods, procedures, treatments, molecules and specific compounds described herein are presently representative of certain embodiments are exemplary and are not intended as limitations on the scope of the invention. Changes therein and other uses will occur to those skilled in the art which are encompassed within the spirit of the invention are defined by the scope of the claims. The listing or discussion of a previously published document in this specification should not necessarily be taken as an acknowledgement that the document is part of the state of the art or is common general knowledge.

The invention illustratively described herein may suitably be practiced in the absence of any element or elements, limitation or limitations, not specifically disclosed herein. Thus, for example, the terms “comprising”, “including,” containing”, etc. shall be read expansively and without limitation. Additionally, the terms and expressions employed herein have been used as terms of description and not of limitation, and there is no intention in the use of such terms and expressions of excluding any equivalents of the features shown and described or portions thereof, but it is recognized that various modifications are possible within the scope of the invention claimed. Thus, it should be understood that although the present invention has been specifically disclosed by exemplary embodiments and optional features, modification and variation of the inventions embodied herein may be resorted to by those skilled in the art, and that such modifications and variations are considered to be within the scope of this invention.

The invention has been described broadly and generically herein. Each of the narrower species and sub-generic groupings falling within the generic disclosure also form part of the invention. This includes the generic description of the invention with a proviso or negative limitation removing any subject matter from the genus, regardless of whether or not the excised material is specifically recited herein.

Other embodiments are within the following claims. In addition, where features or aspects of the invention are described in terms of Markush groups, those skilled in the art will recognize that the invention is also thereby described in terms of any individual member or subgroup of members of the Markush group.

1. A PCR-method comprising conducting a simultaneous amplification step with at least primer nucleotide sequences for SARS-CoV-2 RdRP, at least primer nucleotide sequences for SARS-CoV-2 E gene and at least primer nucleotide sequences for human RNase P, wherein said PCR-method is a multiplex PCR, preferably a multiplex real-time RT-PCR, further preferably said multiplex real-time RT-PCR method comprising conducting said simultaneous amplification step with at least one (e.g., two different) probe specific for the SARS-CoV-2 RdRP, at least one (e.g., two different) probe specific for the SARS-CoV-2 E and at least one (e.g., two different) probe specific for the human RNase P.

2. A PCR-method comprising
(i) a first PCR-method comprising conducting a simultaneous amplification step with at least primer nucleotide sequences for SARS-CoV-2 RdRP and at least primer nucleotide sequences for human RNase P, preferably with at least one (e.g., two different) probe specific for the SARS-CoV-2 RdRP and at least one (e.g., two different) probe specific for human RNase P; and/or
(ii) a second PCR-method comprising conducting a simultaneous amplification step with at least primer nucleotide sequences for SARS-CoV-2 E gene and at least primer nucleotide sequences for human RNase P, preferably with at least one (e.g., two different) probe specific for the SARS-CoV-2 E and at least one (e.g., two different) probe specific for the human RNase P,
wherein the same source material suspected to comprise SARS-CoV-2 nucleic acids is used for the PCR of (i) and (ii), wherein said first and second PCR-methods are multiplex PCR methods, preferably multiplex real-time RT-PCR methods.

3. A PCR-method comprising
(i) a first PCR-method comprising conducting a simultaneous amplification step with at least primer nucleotide sequences for SARS-CoV2 E gene and at least primer nucleotide sequences for a unique spike RNA, preferably with at least one (e.g., two different) probe specific for the SARS-CoV-2 E and at least one (e.g., two different) probe specific for the unique spike RNA;
(ii) a second PCR-method comprising conducting a simultaneous amplification step with at least primer nucleotide sequences for SARS-CoV-2 RdRP gene and at least primer nucleotide sequences for human RNase P, preferably with at least one (e.g., two different) probe specific for the SARS-CoV-2 RdRP and at least one (e.g., two different) probe specific for the human RNase P; and
(iii) a third PCR-method comprising conducting a simultaneous amplification step with at least primer nucleotide sequences for SARS-CoV-2 RdRP gene, at least primer nucleotide sequences for SARS-CoV-2 E gene, at least primer nucleotide sequences for human RNase P and at least primer nucleotide sequences for a unique spike RNA, preferably with at least one (e.g., two different) probe specific for the SARS-CoV-2 RdRP, at least one (e.g., two different) probe specific for the SARS-CoV-2 E and at least one probe specific for the human RNase P and at least one probe specific for the unique spike RNA;
wherein the same source material suspected to comprise SARS-CoV-2 nucleic acids is used for the PCR-method of (i) and (ii), and
wherein the PCR-method of (iii) further comprises as positive control a ribonucleic acid for SARS-CoV-2 RdRP gene, a ribonucleic acid for SARS-CoV-2 E gene, a ribonucleic acid for human RNase P and a ribonucleic acid for unique spike RNA, wherein said first, second and third PCR-methods are multiplex PCR methods, preferably multiplex real-time RT-PCR methods.

4. A PCR-method comprising:
(i) a first PCR-method comprising conducting a simultaneous amplification step with at least primer nucleotide sequences for SARS-CoV2 E gene, at least primer nucleotide sequences for IAV PB1 gene and at least primer nucleotide sequences for human RNase P, preferably with at least one (e.g., two) probe specific for SARS-CoV-2 E, at least one (e.g., two) probe specific for IAV PB1 gene and at least one probe specific for human RNase P;
(ii) a second PCR-method comprising conducting a simultaneous amplification step with at least primer nucleotide sequences for SARS-CoV-2 RdRP gene and at least primer nucleotide sequences for human RNase P and with at least primer nucleotide sequences for IBV PA gene, preferably with at least one (e.g., two, e.g., two partially overlapping but non-identical) probe specific for SARS-CoV-2 RdRP, at least one probe specific for human RNase P, and at least one (e.g., two) probe specific for IBV PA gene.
wherein the same source material suspected to comprise SARS-CoV-2 nucleic acids is used for the PCR-method of (i) and (ii), wherein said first and second PCR-methods are multiplex PCR methods, preferably multiplex real-time RT-PCR methods.

5. A PCR-method comprising conducting a simultaneous amplification step with at least primer nucleotide sequences for SARS-CoV-2 RdRP, at least primer nucleotide sequences for SARS-CoV-2 E gene and at least primer nucleotide sequences for human RNase P, at least primer nucleotide sequences for IBV PA gene and at least primer nucleotide sequences for IAV PB1 gene, wherein said PCR-method is a multiplex PCR method, preferably a multiplex real-time RT-PCR, further preferably with at least one (e.g., two) probe specific for SARS-CoV-2 E, at least one (e.g., two) probe specific for IAV PB1 gene, at least one probe specific for human RNase P, at least one (e.g., two, e.g., two partially overlapping, but non-identical) probe specific for SARS-CoV-2 RdRP and at least one probe specific for IBV PA gene.

6. The PCR-method of any one of the preceding claims, wherein:
i) for the amplification step for IAV PB1 at least the primer nucleotide sequences set forth in SEQ ID NOs: 83-84 or 121-128 are used, preferably SEQ ID NOs: 83-84; further preferably with at least one probe selected from the group consisting of: SEQ ID NOs: 85, 129-131, preferably SEQ ID NO: 85; and/or
ii) for the amplification step for IBV PA at least the primer nucleotide sequences set forth in SEQ ID NOs: 86-87 or 132-144 are used, preferably SEQ ID NOs: 86-87; further preferably with at least one probe selected from the group consisting of: SEQ ID NOs: 88, 145-153, preferably SEQ ID NO: 88.

7. The PCR-method of any one of the preceding claims, wherein for the amplification step for SARS-CoV-2 RdRP at least the primer nucleotide sequences set forth in SEQ ID NOs: 1 and 2, SEQ ID NOs: 27 and 28, SEQ ID NOs: 33 and 34, SEQ ID NOs: 43 and 44, SEQ ID NOs: 54 and 55, SEQ ID NOs: 65 and 66, SEQ ID NOs: 76 and 77, SEQ ID NOs: 109 and 110, SEQ ID NOs: 109 and 111 or SEQ ID NOs: 109 and 112 are used, preferably with at least one (e.g., two) probe selected from the group consisting of: SEQ ID NOs: 29, 35, 45, 46, 56, 57, 67, 68, 78, 79 and 113-120, further preferably with two partially overlapping (e.g., partially comprising the identical sequence, but non-identical) probes selected from the group consisting of: SEQ ID NOs: 45-46, SEQ ID NOs: 56-57, 67-68, 78-79.

8. The PCR-method of any one of the preceding claims, wherein for the amplification step for SARS-CoV-2 E gene at least the primer nucleotide sequences set forth in SEQ ID NOs: 4 or 22 and 5, SEQ ID NOs: 24-25, SEQ ID NOs: 30-31, SEQ ID NOs: 39-40, 50-51, 61-62, 72-73, or any one of 89-101 with 102 are used, preferably with at least one (e.g., two) probe selected from the group consisting of: SEQ ID NOs: 26, 32, 41, 42, 52, 53, 63, 64, 74, 75, 103-108, further preferably with two probes selected from the group consisting of: SEQ ID NOs: 41-42, SEQ ID NOs: 52-53, 63-64, 74-75.

9. The PCR-method of any one of the preceding claims, wherein for the amplification step for human RNase P at least the primer nucleotide sequences set forth in SEQ ID NOs: 7 and 8, or SEQ ID NOs: 36-37, 47-48, 58-59, 69-70, 80-81 or 154-155 are used, preferably with at least one probe selected from the group consisting of: SEQ ID NOs: 38, 49, 60, 71, 82 or 156.

10. The PCR-method of any one of the preceding claims, wherein for the amplification step for unique spike RNA at least the primer nucleotide sequences set forth in SEQ ID NOs: 17 and 18 are used.

11. The PCR-method of any one of the preceding claims, further comprising:
i) a probe comprising the nucleotide sequence set forth in SEQ ID NO: 3, a probe comprising the nucleotide sequence set forth in SEQ ID NO: 6, a probe comprising the nucleotide sequence set forth in SEQ ID NO: 9, a probe comprising the nucleotide sequence set forth in SEQ ID NO: 19; and/or
ii) a probe comprising the nucleotide sequence set forth in SEQ ID NO: 26, a probe comprising the nucleotide sequence set forth in SEQ ID NO: 29, preferably in combination with primer nucleotide sequences set forth in SEQ ID NOs: 24-25 and 27-28; and/or
iii) a probe comprising the nucleotide sequence set forth in SEQ ID NO: 32, a probe comprising the nucleotide sequence set forth in SEQ ID NO: 35, a probe comprising the nucleotide sequence set forth in SEQ ID NO: 38, preferably in combination with primer nucleotide sequences set forth in SEQ ID NOs: 30-31, 33-34 and 36-37; and/or
iv) a probe comprising the nucleotide sequence set forth in SEQ ID NO: 41, a probe comprising the nucleotide sequence set forth in SEQ ID NO: 42, a probe comprising the nucleotide sequence set forth in SEQ ID NO: 45, a probe comprising the nucleotide sequence set forth in SEQ ID NO: 46, a probe comprising the nucleotide sequence set forth in SEQ ID NO: 49, preferably in combination with primer nucleotide sequences set forth in SEQ ID NOs: 39-40, 43-44, 47-48; and/or
v) a probe comprising the nucleotide sequence set forth in SEQ ID NO: 52, a probe comprising the nucleotide sequence set forth in SEQ ID NO: 53, a probe comprising the nucleotide sequence set forth in SEQ ID NO: 56, a probe comprising the nucleotide sequence set forth in SEQ ID NO: 57, a probe comprising the nucleotide sequence set forth in SEQ ID NO: 60, preferably in combination with primer nucleotide sequences set forth in SEQ ID NOs: 50-51, 54-55, 58-59; and/or
vi) a probe comprising the nucleotide sequence set forth in SEQ ID NO: 63, a probe comprising the nucleotide sequence set forth in SEQ ID NO: 64, a probe comprising the nucleotide sequence set forth in SEQ ID NO: 67, a probe comprising the nucleotide sequence set forth in SEQ ID NO: 68, a probe comprising the nucleotide sequence set forth in SEQ ID NO: 71, preferably in combination with primer nucleotide sequences set forth in SEQ ID NOs: 61-62, 65-66 and 69-70; and/or
vii) a probe comprising the nucleotide sequence set forth in SEQ ID NO: 74, a probe comprising the nucleotide sequence set forth in SEQ ID NO: 75, a probe comprising the nucleotide sequence set forth in SEQ ID NO: 78, a probe comprising the nucleotide sequence set forth in SEQ ID NO: 79, a probe comprising the nucleotide sequence set forth in SEQ ID NO: 82, a probe comprising the nucleotide sequence set forth in SEQ ID NO: 85, a probe comprising the nucleotide sequence set forth in SEQ ID NO: 88, preferably in combination with primer nucleotide sequences set forth in SEQ ID NOs: 72-73, 76-77, 80-81, 83-84 and 86-87.

12. The PCR-method of any one of the preceding claims, wherein
a ribonucleic acid having the sequence set forth in SEQ ID NO: 10 is used as positive control,
a ribonucleic acid having the sequence set forth in SEQ ID NO: 11 or 23 is used as positive control,
a ribonucleic acid having the sequence set forth in SEQ ID NO: 12 is used as positive control, optionally said positive control further comprises nucleic acid decoys in the form of baker's yeast tRNA and/or salmon sperm DNA.

13. The PCR-method of any one of the preceding claims, wherein a ribonucleic acid having the sequence set forth in SEQ ID NO: 20 is used as spike RNA.

14. A kit comprising at least the primer nucleotide sequences set forth in SEQ ID NOs: 1 and 2 or SEQ ID NOs: 27-28, SEQ ID NOs: 33-34, SEQ ID NOs: 43-44, SEQ ID NOs: 54-55, SEQ ID NOs: 65-66, SEQ ID NOs: 76-77, SEQ ID NOs: 109-110, SEQ ID NOs: 109 and 111 or SEQ ID NOs: 109 and 112, optionally at least one (e.g., two different) probe comprising the nucleotide sequence selected from the group consisting of: SEQ ID NO: 3, SEQ ID NOs: 29, 35, 45, 46, 56, 57, 67, 68, 78, 79 or 113-120, preferably comprising two (e.g., partially overlapping, but non-identical) probes selected from the group consisting of: SEQ ID NOs: 45-46, SEQ ID NOs: 56-57, 67-68, 78-79 and further optionally means for carrying out a PCR amplification step, preferably said kit is a multiplex PCR kit, preferably said multiplex PCR kit is a multiplex real-time RT-PCR kit.

15. The kit of any one of the preceding claims, further comprising at least the primer nucleotide sequences set forth in SEQ ID NOs: 4 or 22 and 5 or SEQ ID NOs: 24-25, SEQ ID NOs: 30-31, SEQ ID NOs: 39-40, 50-51, 61-62, 72-73 or any one of 89-101 in combination with 102, and optionally at least one (e.g., two different) probe comprising the nucleotide sequence selected from the group consisting of: SEQ ID NO: 6, SEQ ID NOs: 26, 32, 41, 42, 52, 53, 63, 64, 74, 75, 103-108, preferably comprising two (e.g., different) probes selected from the group consisting of: SEQ ID NOs: 41-42, SEQ ID NOs: 52-53, 63-64, 74-75.

16. The kit of any one of the preceding claims, further comprising at least the primer nucleotide sequences set forth in SEQ ID NOs: 7 and 8 or SEQ ID NOs: 36-37, 47-48, 58-59, 69-70, 80-81 or 154-155, and optionally at least one (e.g., two, e.g., two different) probe comprising the nucleotide sequence selected from the group consisting of: SEQ ID NO: 9, SEQ ID NOs: 38, 49, 60, 71, 82 or 156.

17. The kit of any one of the preceding claims, further comprising at least the primer nucleotide sequences set forth in SEQ ID NOs: 83-84 or 121-128, preferably in SEQ ID NOs: 83-84, optionally at least one (e.g., two, e.g., two different) probe selected from the group consisting of: SEQ ID NOs: 85, 129-131, further preferably SEQ ID NO: 85.

18. The kit of any one of the preceding claims, further comprising at least the primer nucleotide sequences set forth in SEQ ID NOs: 86-87 or 132-144 are used, preferably SEQ ID NOs: 86-87, optionally at least one (e.g., two, e.g., two different) probe selected from the group consisting of: SEQ ID NOs: 88, 145-153, further preferably SEQ ID NO: 88

19. Use of the primer nucleotide sequences, wherein said primer sequences:
(a) set forth in SEQ ID NOs: 1-2 or SEQ ID NOs: 27-28, SEQ ID NOs: 33-34, SEQ ID NOs: 43-44, SEQ ID NOs: 54-55, SEQ ID NOs: 65-66, SEQ ID NOs: 76-77, SEQ ID NOs: 109-110, SEQ ID NOs: 109 and 111 SEQ ID NOs: 109 and 112, preferably with at least one (e.g., two) probe selected from the group consisting of: SEQ ID NOs: 29, 35, 45, 46, 56, 57, 67, 68, 78, 79 and 113-120, further preferably with two partially overlapping probes (e.g., non-identical probes) selected from the group consisting of: SEQ ID NOs: 45-46, SEQ ID NOs: 56-57, 67-68, 78-79; and/or
(b) set forth in SEQ ID NOs: 4 or 22 and 5, or SEQ ID NOs: 24-25, SEQ ID NOs:
30-31, SEQ ID NOs: 39-40, 50-51, 61-62, 72-73, any one of 89-101 with 102, preferably with at least one (e.g., two) probe selected from the group consisting of: SEQ ID NOs: 26, 32, 41, 42, 52, 53, 63, 64, 74, 75, 103-108, further preferably with two probes selected from: SEQ ID NOs: 41-42, SEQ ID NOs: 52-53, 63-64, 74-75; and/or
(c) set forth in SEQ ID NOs: 83-84 or 121-128, preferably SEQ ID NOs: 83-84, further preferably with at least one probe selected from the group consisting of: SEQ ID NOs: 85, 129-131, most preferably SEQ ID NO: 85;
(d) set forth in SEQ ID NOs: 86-87 or 132-144, preferably SEQ ID NOs: 86-87, further preferably with at least one probe selected from the group consisting of: SEQ ID NOs: SEQ ID NOs: 88, 145-153, preferably SEQ ID NO: 88;
wherein said sequences of (a), (b), (c) and (d) are used alone or in combination with one another for one or more of the following:
(i) in vitro detection (e.g., simultaneous, e.g., multiplexed detection) of SARS-CoV-2 and/or IAV and/or IBV in a sample,
(ii) in vitro detection (e.g., simultaneous, e.g., multiplexed detection) of an infection of a subject with SARS-CoV-2 and/or IAV and/or IBV,
(iii) in vitro detection (e.g., simultaneous, e.g., multiplexed detection) of a contamination of a blood sample with SARS-CoV-2 and/or IAV and/or IBV, or
(iv) monitoring (e.g., simultaneous, e.g., multiplexed monitoring) the therapy of SARS-CoV-2 and/or IAV and/or IBV in vitro,
wherein said use is the use for multiplex PCR detection, preferably a multiplex real-time RT-PCR detection;
(v) for/in the PCR-method according of any one of the preceding claims, preferably said method is an in vitro or ex vivo method.

20. The PCR-method, kit or use according to any one of the preceding claims, wherein said primer and/or probe nucleotide sequence/s comprising one or more (e.g., 2, 3 or 4) Locked Nucleic Acids (LNA)-modified nucleotides (e.g., LNA is a synthetic nucleic acid analogue containing a bridged, bicyclic sugar moiety, e.g., a methylene linkage between the 2′ oxygen and the 4′ carbon of the ribose ring), preferably said one or more (e.g., 2, 3 or 4) LNA-modified nucleotides are LNA-modified thymine residues (e.g., LNA-T) and/or LNA-modified adenosine residues (e.g., LNA-A).


SEQUENCE LISTING (162 sequences)

SEQ ID NO: 1 - DNA, 22 bp, Artificial Sequence
  forward primer nucleotide sequence for SARS-CoV -2 RdRP
gtgaaatggtcatgtgtggcgg

SEQ ID NO: 2 - DNA, 27 bp, Artificial Sequence
  reverse primer nucleotide sequence for SARS-CoV -2 RdRP
cgtgacagcttgacaaatgttaaaaac

SEQ ID NO: 3 - DNA, 25 bp, Artificial Sequence
  probe for SARS-CoV-2 RdRP
caggtggaacctcatcaggagatgc

SEQ ID NO: 4 - DNA, 26 bp, Artificial Sequence
  forward primer nucleotide sequence for SARS-CoV -2 E gene
acaggtacgttaatagttaatagcgt

SEQ ID NO: 5 - DNA, 22 bp, Artificial Sequence
  reverse primer nucleotide sequence for SARS-CoV -2 E gene
atattgcagcagtacgcacaca

SEQ ID NO: 6 - DNA, 26 bp, Artificial Sequence
  probe for SARS-CoV-2 E gene
acactagccatccttactgcgcttcg

SEQ ID NO: 7 - DNA, 19 bp, Artificial Sequence
  forward primer nucleotide sequence for human RNAse P
agatttggacctgcgagcg

SEQ ID NO: 8 - DNA, 20 bp, Artificial Sequence
  reverse primer nucleotide sequence for human RNAse P
gagcggctgtctccacaagt

SEQ ID NO: 9 - DNA, 23 bp, Artificial Sequence
  probe for human RNAse P
ttctgacctgaaggctctgcgcg

SEQ ID NO: 10 - RNA, 80 nt, Artificial Sequence
  positive control ribonucleotide for SARS-CoV-2 RdRP
agugaaauggucauguguggcggaccagguggaaccucaucaggagaugccaguuuuuaacauuugucaagcugucacgg

SEQ ID NO: 11 - RNA, 80 nt, Artificial Sequence
  positive control ribonucleotide for SARS-CoV-2 E gene
agacagguacguuaauaguuaauagcguuuacacuagccauccuuacugcgcuucgauugugugcguacugcugcaauau

SEQ ID NO: 12 - RNA, 67 nt, Artificial Sequence
  positive control ribonucleotide for human RNAse P
gcagauuuggaccugcgagcggguucugaccugaaggcucugcgcggacuuguggagacagccgcuc

SEQ ID NO: 13 - DNA, 26 bp, Artificial Sequence
  reverse primer nucleotide sequence for SARS-CoV -2 RdRP
caaatgttaaaaacactattagcata

SEQ ID NO: 14 - DNA, 22 bp, Artificial Sequence
  forward primer nucleotide sequence for SARS-CoV -2 RdRP
gtgaratggtcatgtgtggcgg

SEQ ID NO: 15 - DNA, 26 bp, Artificial Sequence
  reverse primer nucleotide sequence for SARS-CoV -2 RdRP
  misc (3)..(3): R is G or A
  misc (12)..(12): S is G or C
caratgttaaasacactattagcata

SEQ ID NO: 16 - DNA, 26 bp, Artificial Sequence
  probe for SARS-CoV-2 E gene
  misc (9)..(9): W is A or T
  misc (12)..(12): R is G or A
  misc (18)..(18): M is A or C
ccaggtggwacrtcatcmggtgatgc

SEQ ID NO: 17 - DNA, 21 bp, Artificial Sequence
  forward primer nucleotide sequence for unique spike RNA
atgcagtgccacattatgcag

SEQ ID NO: 18 - DNA, 20 bp, Artificial Sequence
  reverse primer nucleotide sequence for unique spike RNA
agcacatgtagtgccactgg

SEQ ID NO: 19 - DNA, 26 bp, Artificial Sequence
  probe for unique spike RNA
ccacggttacatccagtggcactaca

SEQ ID NO: 20 - RNA, 89 nt, Artificial Sequence
  spike RNA
uaugcagugccacauuaugcagugccacgguuacauccaguggcacuacaugugccauuacauuuacauccaguggcacuacaugugcu

SEQ ID NO: 21 - DNA, 89 bp, Artificial Sequence
  synthetic nucleic acid sequence serving as template for spike RNA
tatgcagtgccacattatgcagtgccacggttacatccagtggcactacatgtgccattacatttacatccagtggcactacatgtgct

SEQ ID NO: 22 - DNA, 26 bp, Artificial Sequence
  forward primer nucleotide sequence for SARS-CoV -2 E gene
gtactcattcgtttcggaagagacag

SEQ ID NO: 23 - RNA, 78 nt, Artificial Sequence
  positive control ribonucleotide for SARS-CoV-2 E gene
auguacucauucguuucggaagacaguuacacuagccauccuuacugcgcuucgauugugugcguacugcugcaauau

SEQ ID NO: 24 - DNA, 26 bp, Artificial Sequence
  E_Sarbeco_F3 primer
gtactcattcgtttcggaagagacag

SEQ ID NO: 25 - DNA, 22 bp, Artificial Sequence
  E_Sarbeco_R2 primer
atattgcagcagtacgcacaca

SEQ ID NO: 26 - DNA, 26 bp, Artificial Sequence
  E_Sarbeco_P1 probe
acactagccatccttactgcgcttcg

SEQ ID NO: 27 - DNA, 22 bp, Artificial Sequence
  RdRP_SARSr-F2 primer
gtgaaatggtcatgtgtggcgg

SEQ ID NO: 28 - DNA, 27 bp, Artificial Sequence
  RdRP_SARSr-R2 primer
cgtgacagcttgacaaatgttaaaaac

SEQ ID NO: 29 - DNA, 25 bp, Artificial Sequence
  RdRP_SARSr-P2 probe
caggtggaacctcatcaggagatgc

SEQ ID NO: 30 - DNA, 23 bp, Artificial Sequence
  E_Sarbeco_F7 primer
  misc_feature (2)..(2): T at position 2 is a locked nucleic acid
  misc_feature (4)..(4): T at position 4 is a locked nucleic acid
atgtactcattcgtttcggaaga

SEQ ID NO: 31 - DNA, 22 bp, Artificial Sequence
  E_Sarbeco_R2 primer
atattgcagcagtacgcacaca

SEQ ID NO: 32 - DNA, 26 bp, Artificial Sequence
  E_Sarbeco_P1 probe
acactagccatccttactgcgcttcg

SEQ ID NO: 33 - DNA, 22 bp, Artificial Sequence
  RdRP_SARSr-F2 primer
gtgaaatggtcatgtgtggcgg

SEQ ID NO: 34 - DNA, 27 bp, Artificial Sequence
  RdRP_SARSr-R2 primer
cgtgacagcttgacaaatgttaaaaac

SEQ ID NO: 35 - DNA, 26 bp, Artificial Sequence
  RdRP_SARSr-P8 probe
tcaggagatgccacaactgcttatgc

SEQ ID NO: 36 - DNA, 19 bp, Artificial Sequence
  RNase P Forward primer
agatttggacctgcgagcg

SEQ ID NO: 37 - DNA, 20 bp, Artificial Sequence
  RNase P Reverse primer
gagcggctgtctccacaagt

SEQ ID NO: 38 - DNA, 23 bp, Artificial Sequence
  RNase P Probe 1
ttctgacctgaaggctctgcgcg

SEQ ID NO: 39 - DNA, 23 bp, Artificial Sequence
  E_Sarbeco_F7 primer
  misc_feature (2)..(2): T at position 2 is a locked nucleic acid
  misc_feature (4)..(4): T at position 2 is a locked nucleic acid
atgtactcattcgtttcggaaga

SEQ ID NO: 40 - DNA, 22 bp, Artificial Sequence
  E_Sarbeco_R2 primer
atattgcagcagtacgcacaca

SEQ ID NO: 41 - DNA, 26 bp, Artificial Sequence
  E_Sarbeco_P1 probe
acactagccatccttactgcgcttcg

SEQ ID NO: 42 - DNA, 30 bp, Artificial Sequence
  E_Sarbeco_P2 probe
tagcgtacttctttttcttgctttcgtggt

SEQ ID NO: 43 - DNA, 22 bp, Artificial Sequence
  RdRP_SARSr-F2 probe
gtgaaatggtcatgtgtggcgg

SEQ ID NO: 44 - DNA, 27 bp, Artificial Sequence
  RdRP_SARSr-R2 primer
cgtgacagcttgacaaatgttaaaaac

SEQ ID NO: 45 - DNA, 25 bp, Artificial Sequence
  RdRP_SARSr-P2 probe
caggtggaacctcatcaggagatgc

SEQ ID NO: 46 - DNA, 26 bp, Artificial Sequence
  RdRP_SARSr-P8 probe
tcaggagatgccacaactgcttatgc

SEQ ID NO: 47 - DNA, 19 bp, Artificial Sequence
  RNase P Forward primer
agatttggacctgcgagcg

SEQ ID NO: 48 - DNA, 20 bp, Artificial Sequence
  RNase P Reverse primer
gagcggctgtctccacaagt

SEQ ID NO: 49 - DNA, 23 bp, Artificial Sequence
  RNase P Probe 1
ttctgacctgaaggctctgcgcg

SEQ ID NO: 50 - DNA, 23 bp, Artificial Sequence
  E_Sarbeco_F7 primer
  misc_feature (2)..(2): T at position 2 is a locked nucleic acid
  misc_feature (4)..(4): T at position 4 is a locked nucleic acid
atgtactcattcgtttcggaaga

SEQ ID NO: 51 - DNA, 22 bp, Artificial Sequence
  E_Sarbeco_R2 primer
atattgcagcagtacgcacaca

SEQ ID NO: 52 - DNA, 26 bp, Artificial Sequence
  E_Sarbeco_P1 probe
acactagccatccttactgcgcttcg

SEQ ID NO: 53 - DNA, 30 bp, Artificial Sequence; identical to SEQ ID NO: 42
  E_Sarbeco_P2 probe

SEQ ID NO: 54 - DNA, 22 bp, Artificial Sequence
  RdRP_SARSr-F2 primer
gtgaaatggtcatgtgtggcgg

SEQ ID NO: 55 - DNA, 27 bp, Artificial Sequence
  RdRP_SARSr-R2 primer
cgtgacagcttgacaaatgttaaaaac

SEQ ID NO: 56 - DNA, 25 bp, Artificial Sequence
  RdRP_SARSr-P2 probe
caggtggaacctcatcaggagatgc

SEQ ID NO: 57 - DNA, 26 bp, Artificial Sequence
  RdRP_SARSr-P8 probe
tcaggagatgccacaactgcttatgc

SEQ ID NO: 58 - DNA, 19 bp, Artificial Sequence
  RNase P Forward primer
agatttggacctgcgagcg

SEQ ID NO: 59 - DNA, 20 bp, Artificial Sequence
  RNase P Reverse primer
gagcggctgtctccacaagt

SEQ ID NO: 60 - DNA, 23 bp, Artificial Sequence
  RNase P Probe 1.1
ttctgacctgaaggctctgcgcg

SEQ ID NO: 61 - DNA, 23 bp, Artificial Sequence
  E_Sarbeco_F7 primer
  misc_feature (2)..(2): T at position 2 is a locked nucleic acid
  misc_feature (4)..(4): T at position 4 is a locked nucleic acid
atgtactcattcgtttcggaaga

SEQ ID NO: 62 - DNA, 22 bp, Artificial Sequence
  E_Sarbeco_R2 primer
atattgcagcagtacgcacaca

SEQ ID NO: 63 - DNA, 26 bp, Artificial Sequence
  E_Sarbeco_P1.2 probe
acactagccatccttactgcgcttcg

SEQ ID NO: 64 - DNA, 30 bp, Artificial Sequence; identical to SEQ ID NO: 42
  E_Sarbeco_P2.2 probe

SEQ ID NO: 65 - DNA, 22 bp, Artificial Sequence
  RdRP_SARSr-F2 primer
gtgaaatggtcatgtgtggcgg

SEQ ID NO: 66 - DNA, 27 bp, Artificial Sequence
  RdRP_SARSr-R2 primer
cgtgacagcttgacaaatgttaaaaac

SEQ ID NO: 67 - DNA, 25 bp, Artificial Sequence
  RdRP_SARSr-P2 probe
caggtggaacctcatcaggagatgc

SEQ ID NO: 68 - DNA, 26 bp, Artificial Sequence
  RdRP_SARSr-P8 probe
tcaggagatgccacaactgcttatgc

SEQ ID NO: 69 - DNA, 19 bp, Artificial Sequence
  RNase P Forward primer
agatttggacctgcgagcg

SEQ ID NO: 70 - DNA, 20 bp, Artificial Sequence
  RNase P Reverse primer
gagcggctgtctccacaagt

SEQ ID NO: 71 - DNA, 23 bp, Artificial Sequence
  RNase P Probe 1.2
ttctgacctgaaggctctgcgcg

SEQ ID NO: 72 - DNA, 23 bp, Artificial Sequence
  E_Sarbeco_F7 primer
  misc_feature (2)..(2): T at position 2 is a locked nucleic acid
  misc_feature (4)..(4): T at position 4 is a locked nucleic acid
atgtactcattcgtttcggaaga

SEQ ID NO: 73 - DNA, 22 bp, Artificial Sequence
  E_Sarbeco_R2 primer
atattgcagcagtacgcacaca

SEQ ID NO: 74 - DNA, 26 bp, Artificial Sequence
  E_Sarbeco_P1 probe
acactagccatccttactgcgcttcg

SEQ ID NO: 75 - DNA, 30 bp, Artificial Sequence; identical to SEQ ID NO: 42
  E_Sarbeco_P2 primer

SEQ ID NO: 76 - DNA, 22 bp, Artificial Sequence
  RdRP_SARSr-F2 primer
gtgaaatggtcatgtgtggcgg

SEQ ID NO: 77 - DNA, 27 bp, Artificial Sequence
  RdRP_SARSr-R2 primer
cgtgacagcttgacaaatgttaaaaac

SEQ ID NO: 78 - DNA, 25 bp, Artificial Sequence
  RdRP_SARSr-P2 probe
caggtggaacctcatcaggagatgc

SEQ ID NO: 79 - DNA, 26 bp, Artificial Sequence
  RdRP_SARSr-P8 probe
tcaggagatgccacaactgcttatgc

SEQ ID NO: 80 - DNA, 19 bp, Artificial Sequence
  RNase P Forward primer
agatttggacctgcgagcg

SEQ ID NO: 81 - DNA, 20 bp, Artificial Sequence
  RNase P Reverse primer
gagcggctgtctccacaagt

SEQ ID NO: 82 - DNA, 23 bp, Artificial Sequence
  RNase P Probe 1.2
ttctgacctgaaggctctgcgcg

SEQ ID NO: 83 - DNA, 21 bp, Artificial Sequence
  IAV-F1.1 primer
ttctagcatggtggaggccat

SEQ ID NO: 84 - DNA, 23 bp, Artificial Sequence
  IAV-R1.2 primer
ccgtctgagttcttcaatggtgg

SEQ ID NO: 85 - DNA, 22 bp, Artificial Sequence
  IAV-Probe1.2
tctagggcccggattgatgcca

SEQ ID NO: 86 - DNA, 20 bp, Artificial Sequence
  IBV-F2.1 primer
agtggactcaggaaagtggc

SEQ ID NO: 87 - DNA, 24 bp, Artificial Sequence
  IBV-R2.3 primer
  misc_feature (6)..(6): T at position 6 is a locked nucleic acid
  misc_feature (9)..(9): T at position 9 is a locked nucleic acid
tccatttgttgcattgattgaagc

SEQ ID NO: 88 - DNA, 26 bp, Artificial Sequence
  IBV-Probe2.3
  misc_feature (4)..(4): A at position 4 is a locked nucleic acid
  misc_feature (11)..(11): A at position 11 is a locked nucleic acid
tccaaatgaaatggggaatggaagct

SEQ ID NO: 89 - DNA, 26 bp, Artificial Sequence
  E_Sarbeco_F1 primer
acaggtacgttaatagttaatagcgt

SEQ ID NO: 90 - DNA, 24 bp, Artificial Sequence
  E_Sarbeco_F2 primer
ctcattcgtttcggaagagacagg

SEQ ID NO: 91 - DNA, 26 bp, Artificial Sequence
  E_Sarbeco_F3 primer
gtactcattcgtttcggaagagacag

SEQ ID NO: 92 - DNA, 22 bp, Artificial Sequence
  E_Sarbeco_F4 primer
cgtttcggaagagacaggtacg

SEQ ID NO: 93 - DNA, 26 bp, Artificial Sequence
  E_Sarbeco_F5 primer
tgtactcattcgtttcggaagagaca

SEQ ID NO: 94 - DNA, 27 bp, Artificial Sequence
  E_Sarbeco_F6 primer
tgtactcattcgtttcggaagagacag

SEQ ID NO: 95 - DNA, 23 bp, Artificial Sequence
  E_Sarbeco_F7 primer
  misc_feature (2)..(2): T at position 2 is a locked nucleic acid
  misc_feature (4)..(4): T at position 4 is a locked nucleic acid
atgtactcattcgtttcggaaga

SEQ ID NO: 96 - DNA, 24 bp, Artificial Sequence
  E_Sarbeco_F8 primer
  misc_feature (2)..(2): T at position 2 is a locked nucleic acid
  misc_feature (4)..(4): T at position 4 is a locked nucleic acid
atgtactcattcgtttcggaagac

SEQ ID NO: 97 - DNA, 20 bp, Artificial Sequence
  E_Sarbeco_F9 primer
  misc_feature (4)..(4): T at position 4 is a locked nucleic acid
  misc_feature (7)..(7): T at position 7 is a locked nucleic acid
  misc_feature (10)..(10): T at position 10 is a locked nucleic acid
atgtactcattcgtttcgga

SEQ ID NO: 98 - DNA, 20 bp, Artificial Sequence
  E_Sarbeco_F10 primer
  misc_feature (2)..(2): T at position 2 is a locked nucleic acid
  misc_feature (4)..(4): T at position 4 is a locked nucleic acid
  misc_feature (7)..(7): T at position 7 is a locked nucleic acid
atgtactcattcgtttcgga

SEQ ID NO: 99 - DNA, 20 bp, Artificial Sequence
  E_Sarbeco_F11 primer
  misc_feature (4)..(4): T at position 4 is a locked nucleic acid
  misc_feature (10)..(10): T at position 10 is a locked nucleic acid
  misc_feature (16)..(16): T at position 16 is a locked nucleic acid
atgtactcattcgtttcgga

SEQ ID NO: 100 - DNA, 20 bp, Artificial Sequence
  E_Sarbeco_F12 primer
  misc_feature (2)..(2): T at position 2 is a locked nucleic acid
  misc_feature (11)..(11): T at position 11 is a locked nucleic acid
  misc_feature (16)..(16): T at position 16 is a locked nucleic acid
atgtactcattcgtttcgga

SEQ ID NO: 101 - DNA, 20 bp, Artificial Sequence
  E_Sarbeco_F13 primer
  misc_feature (4)..(4): T at position 4 is a locked nucleic acid
  misc_feature (10)..(10): T at position 10 is a locked nucleic acid
  misc_feature (15)..(15): T at position 15 is a locked nucleic acid
atgtactcattcgtttcgga

SEQ ID NO: 102 - DNA, 22 bp, Artificial Sequence
  E_Sarbeco_R2 primer
atattgcagcagtacgcacaca

SEQ ID NO: 103 - DNA, 26 bp, Artificial Sequence
  E_Sarbeco_P1 probe
acactagccatccttactgcgcttcg

SEQ ID NO: 104 - DNA, 30 bp, Artificial Sequence; identical to SEQ ID NO: 42
  E_Sarbeco_P2 probe

SEQ ID NO: 105 - DNA, 26 bp, Artificial Sequence
  Sarbeco_P1rev probe
cgaagcgcagtaaggatggctagtgt

SEQ ID NO: 106 - DNA, 30 bp, Artificial Sequence; identical to SEQ ID NO: 42
  E_Sarbeco_P3 probe
  misc_feature (14)..(14): T at position 14 is a locked nucleic acid

SEQ ID NO: 107 - DNA, 30 bp, Artificial Sequence; identical to SEQ ID NO: 42
  E_Sarbeco_P4 probe
  misc_feature (13)..(13): T at position 13 is a locked nucleic acid
  misc_feature (15)..(15): T at position 15 is a locked nucleic acid

SEQ ID NO: 108 - DNA, 30 bp, Artificial Sequence; identical to SEQ ID NO: 42
  E_Sarbeco_P5 probe
  misc_feature (14)..(14): T at position 14 is a locked nucleic acid

SEQ ID NO: 109 - DNA, 22 bp, Artificial Sequence
  RdRP_SARSr-F2 primer
gtgaaatggtcatgtgtggcgg

SEQ ID NO: 110 - DNA, 27 bp, Artificial Sequence
  RdRP_SARSr-R2 primer
cgtgacagcttgacaaatgttaaaaac

SEQ ID NO: 111 - DNA, 27 bp, Artificial Sequence
  RdRP_SARSr-R4 primer
  misc_feature (21)..(21): T at position 21 is a locked nucleic acid
cgtgacagcttgacaaatgttaaaaac

SEQ ID NO: 112 - DNA, 27 bp, Artificial Sequence
  RdRP_SARSr-R5 primer
  misc_feature (18)..(18): T at position 18 is a locked nucleic acid
  misc_feature (21)..(21): T at position 21 is a locked nucleic acid
cgtgacagcttgacaaatgttaaaaac

SEQ ID NO: 113 - DNA, 25 bp, Artificial Sequence
  RdRP_SARSr-P2 probe
caggtggaacctcatcaggagatgc

SEQ ID NO: 114 - DNA, 25 bp, Artificial Sequence
  RdRP_SARSr-P3 probe
caggtggaacctcatcaggagatgc

SEQ ID NO: 115 - DNA, 25 bp, Artificial Sequence
  RdRP_SARSr-P4 probe
gtggaacctcatcaggagatgccac

SEQ ID NO: 116 - DNA, 25 bp, Artificial Sequence
  RdRP_SARSr-P5 probe
gtggaacctcatcaggagatgccac

SEQ ID NO: 117 - DNA, 26 bp, Artificial Sequence
  RdRP_SARSr-P6 probe
ggaacctcatcaggagatgccacaac

SEQ ID NO: 118 - DNA, 26 bp, Artificial Sequence
  RdRP_SARSr-P7 probe
ggaacctcatcaggagatgccacaac

SEQ ID NO: 119 - DNA, 26 bp, Artificial Sequence
  RdRP_SARSr-P8 probe
tcaggagatgccacaactgcttatgc

SEQ ID NO: 120 - DNA, 26 bp, Artificial Sequence
  RdRP_SARSr-P9 probe
tcaggagatgccacaactgcttatgc

SEQ ID NO: 121 - DNA, 21 bp, Artificial Sequence
  IAV-F1.1 primer
ttctagcatggtggaggccat

SEQ ID NO: 122 - DNA, 21 bp, Artificial Sequence
  IAV-F1.2 primer
atttctagcatggtggaggcc

SEQ ID NO: 123 - DNA, 20 bp, Artificial Sequence
  IAV-F2.1 primer
aatcccctgaatccctttgt

SEQ ID NO: 124 - DNA, 20 bp, Artificial Sequence
  IAV-F2.2 primer
  misc_feature (2)..(2): A at position 2 is a locked nucleic acid
  misc_feature (11)..(11): A at position 11 is a locked nucleic acid
aatcccctgaatccctttgt

SEQ ID NO: 125 - DNA, 20 bp, Artificial Sequence
  IAV-F2.3 primer
  misc_feature (3)..(3): T at position 3 is a locked nucleic acid
  misc_feature (12)..(12): T at position 12 is a locked nucleic acid
aatcccctgaatccctttgt

SEQ ID NO: 126 - DNA, 22 bp, Artificial Sequence
  IAV-R1.1 primer
cgtctgagttcttcaatggtgg

SEQ ID NO: 127 - DNA, 23 bp, Artificial Sequence
  IAV-R1.2 primer
ccgtctgagttcttcaatggtgg

SEQ ID NO: 128 - DNA, 18 bp, Artificial Sequence
  IAV-R2.1 primer
tctggcatcaatccgggc

SEQ ID NO: 129 - DNA, 21 bp, Artificial Sequence
  IAV-Probe1.1
agggcccggattgatgccaga

SEQ ID NO: 130 - DNA, 22 bp, Artificial Sequence
  IAV-Probe1.2
tctagggcccggattgatgcca

SEQ ID NO: 131 - DNA, 26 bp, Artificial Sequence
  IAV-Probe1.3
  misc_feature (6)..(6): T at position 6 is a locked nucleic acid
  misc_feature (13)..(13): A at position 13 is a locked nucleic acid
  misc_feature (19)..(19): T at position 19 is a locked nucleic acid
  misc_feature (22)..(22): A at position 22 is a locked nucleic acid
aagagttctctgagatcatgaagatc

SEQ ID NO: 132 - DNA, 20 bp, Artificial Sequence
  IBV-F1.1 primer
aagcaatgccagcatgggaa

SEQ ID NO: 133 - DNA, 21 bp, Artificial Sequence
  IBV-F1.2 primer
  misc_feature (3)..(3): A at position 3 is a locked nucleic acid
  misc_feature (8)..(8): A at position 8 is a locked nucleic acid
tgaatgaaagcaatgccagca

SEQ ID NO: 134 - DNA, 20 bp, Artificial Sequence
  IBV-F1.3 primer
  misc_feature (2)..(2): A at position 2 is a locked nucleic acid
  misc_feature (6)..(6): A at position 6 is a locked nucleic acid
gaatgaaagcaatgccagca

SEQ ID NO: 135 - DNA, 20 bp, Artificial Sequence
  IBV-F2.1 primer
agtggactcaggaaagtggc

SEQ ID NO: 136 - DNA, 23 bp, Artificial Sequence
  IBV-F2.2 primer
  misc_feature (3)..(3): T at position 3 is a locked nucleic acid
  misc_feature (9)..(9): T at position 9 is a locked nucleic acid
tttaggattggctccctatttgt

SEQ ID NO: 137 - DNA, 24 bp, Artificial Sequence
  IBV-F2.3 primer
  misc_feature (3)..(3): T at position 3 is a locked nucleic acid
  misc_feature (9)..(9): T at position 9 is a locked nucleic acid
tttaggattggctccctatttgtg

SEQ ID NO: 138 - DNA, 24 bp, Artificial Sequence
  IBV-F2.4 primer
actgtgtttaggattggctcccta

SEQ ID NO: 139 - DNA, 22 bp, Artificial Sequence
  IBV-R1.1 primer
gggagccaatcctaaacacagt

SEQ ID NO: 140 - DNA, 20 bp, Artificial Sequence
  IBV-R1.2 primer
  misc_feature (2)..(2): A at position 2 is a locked nucleic acid
  misc_feature (7)..(7): A at position 7 is a locked nucleic acid
gagccaatcctaaacacagt

SEQ ID NO: 141 - DNA, 20 bp, Artificial Sequence
  IBV-R1.3 primer
  misc_feature (2)..(2): A at position 2 is a locked nucleic acid
  misc_feature (7)..(7): A at position 7 is a locked nucleic acid
  misc_feature (12)..(12): A at position 12 is a locked nucleic acid
gagccaatcctaaacacagt

SEQ ID NO: 142 - DNA, 24 bp, Artificial Sequence
  IBV-R2.1 primer
tccatttgttgcattgattgaagc

SEQ ID NO: 143 - DNA, 25 bp, Artificial Sequence
  IBV-R2.2 primer
tccatttgttgcattgattgaagca

SEQ ID NO: 144 - DNA, 24 bp, Artificial Sequence
  IBV-R2.3 primer
  misc_feature (6)..(6): T at position 6 is a locked nucleic acid
  misc_feature (9)..(9): T at position 9 is a locked nucleic acid
tccatttgttgcattgattgaagc

SEQ ID NO: 145 - DNA, 23 bp, Artificial Sequence
  IBV-Probe1.1
agagtggactcaggaaagtggcc

SEQ ID NO: 146 - DNA, 24 bp, Artificial Sequence
  IBV-Probe1.2
cagagtggactcaggaaagtggcc

SEQ ID NO: 147 - DNA, 26 bp, Artificial Sequence
  IBV-Probe2.1
tccaaatgaaatggggaatggaagct

SEQ ID NO: 148 - DNA, 29 bp, Artificial Sequence
  IBV-Probe2.2
agatccaaatgaaatggggaatggaagct

SEQ ID NO: 149 - DNA, 26 bp, Artificial Sequence
  IBV-Probe2.3
  misc_feature (4)..(4): A at position 4 is a locked nucleic acid
  misc_feature (11)..(11): A at position 11 is a locked nucleic acid
tccaaatgaaatggggaatggaagct

SEQ ID NO: 150 - DNA, 29 bp, Artificial Sequence
  IBV-Probe2.4
tgcagagtgaatggcacaaataagatcca

SEQ ID NO: 151 - DNA, 28 bp, Artificial Sequence
  IBV-Probe2.5
tgccgagtgaatggcacaaataagatcc

SEQ ID NO: 152 - DNA, 30 bp, Artificial Sequence
  IBV-Probe2.6
actgtgtttaggattggctccctatttgtg

SEQ ID NO: 153 - DNA, 25 bp, Artificial Sequence
  IBV-Probe2.7
  misc_feature (4)..(4): T at position 4 is a locked nucleic acid
  misc_feature (14)..(14): T at position 14 is a locked nucleic acid
  misc_feature (24)..(24): T at position 24 is a locked nucleic acid
ctgtgtttaggattggctccctatt

SEQ ID NO: 154 - DNA, 19 bp, Artificial Sequence
  RNAse P Forward primer
agatttggacctgcgagcg

SEQ ID NO: 155 - DNA, 20 bp, Artificial Sequence
  RNAse P Reverse primer
gagcggctgtctccacaagt

SEQ ID NO: 156 - DNA, 23 bp, Artificial Sequence
  RNAse P Probe
ttctgacctgaaggctctgcgcg

SEQ ID NO: 157 - DNA, 23 bp, Artificial Sequence
  SARS-CoV-2 S gene - F1
  misc_feature (4)..(4): T at position 4 is LNA
  misc_feature (6)..(6): T at position 6 is LNA
tcttttccaatgttacttggttc

SEQ ID NO: 158 - DNA, 24 bp, Artificial Sequence
  SARS-CoV-2 S gene - R1
agtagggactgggtcttcgaatct

SEQ ID NO: 159 - DNA, 23 bp, Artificial Sequence
  B.1.1.7 - F3
  misc_feature (3)..(3): T at position 3 is LNA
  misc_feature (7)..(7): T at position 7 is LNA
gttacttggttccatgctatctc

SEQ ID NO: 160 - DNA, 26 bp, Artificial Sequence
  B.1.1.7 - R36
  misc_feature (5)..(5): T at position 5 is LNA
  misc_feature (8)..(8): T at position 8 is LNA
  misc_feature (19)..(19): T at position 19 is LNA
caacttttgttgtttttgtggtaagc

SEQ ID NO: 161 - DNA, 26 bp, Artificial Sequence
  SARS-CoV-2 S gene / B.1.1.7 - P3 probe
  misc_feature (19)..(19): T at position 19 is LNA
  misc_feature (22)..(22): T at position 22 is LNA
agaggtttgataaccctgtcctacca

SEQ ID NO: 162 - DNA, 26 bp, Artificial Sequence
  SARS-CoV-2 S gene / B.1.1.7- P4 probe
  misc_feature (2)..(2): T at position 2 is LNA
  misc_feature (21)..(21): T at position 21 is LNA
tttgcttccactgagaagtctaacat